(19) United States
(12) Patent Application Publication
(10) Pub. No.: US 2006/0293696 A1
(43) Pub. Date: Dec. 28, 2006

(54) Retrieval catheter

(75) Inventors: Gary Fahey, Rossacahill (IE); Ronald Kelly, Athlone (IE); Patrick Griffin, Catlegar (IE); Eamon Brady, Elphin (IE); David Vale, Contarf (IE); John Neilan, Gort (IE)

Correspondence Address: RISSMAN JOBSE HENDRICKS & OLIVERIO, LLP, ONE STATE STREET, SUITE 800, BOSTON, MA, 02109, US

(73) Assignee: Salviac Limited, Dublin (IE)

(21) Appl. No.: 11/407,355

(22) Filed: Apr. 18, 2006

Related U.S. Application Data
(60) Provisional application No. 60/672,300, filed on Apr. 18, 2005.

(51) Int. Cl. A61B 17/22

(52) U.S. Cl. 606/127

(57) ABSTRACT

A retrieval catheter (200) comprises a catheter shaft (2) and a retrieval catheter tip at the distal end of the shaft (2). The tip comprises an expandable tip body (201) and two reinforcement columns (6, 7) extending along the tip body (201) embedded within the tip body (201). The tip body (201) comprises a stiff, distal, radiopaque segment (203) and a soft, proximal segment (202). The distal segment (203) has a distal nose part (205) extending distally of the reinforcement columns (6, 7). Along the distal nose part (205), the wall thickness of the tip body (201) tapers distally from a smaller wall thickness to a larger wall thickness, and the internal diameter tapers distally from a larger diameter to a smaller diameter.

### RELATED APPLICATION

This application claims the benefit of U.S. Patent Application Ser. No. 60/672,300, filed Apr. 18, 2005, the entire teachings of which are incorporated herein by reference.

### INTRODUCTION

This invention relates to a retrieval catheter and to a retrieval catheter tip into which an object may be retrieved.

It is known to insert a retrieval catheter into a body passageway of a patient, and to advance the retrieval catheter through the body passageway to retrieve an object, such as an embolic protection filter, from the passageway into the retrieval catheter. During advancement of the retrieval catheter through the body passageway, the retrieval catheter tip must present a low profile to facilitate advancement through the potentially narrow and/or tortuous passageway, and also to avoid causing damage or discomfort to the patient.

Conventional retrieval catheters suffer from a number of problems. Generally the object to be retrieved from the body passageway has a larger size than the retrieval catheter tip. It is therefore difficult to retrieve the relatively large object into the relatively small retrieval catheter tip. In addition, retrieval of the large object into the retrieval catheter tip is prone to cause buckling of the retrieval catheter tip due to the large compressive forces acting on the tip during the retrieval process.

This invention is aimed at providing an improved retrieval catheter which addresses at least some of these problems.

### STATEMENTS OF INVENTION

According to the invention there is provided a retrieval catheter tip comprising:—
an expandable tip body into which an object may be retrieved;
the tip body comprising a relatively stiff segment and a relatively soft segment; and
at least one reinforcement element for reinforcing the tip body.

In one embodiment of the invention the tip body is at least partially of a radiopaque material. The stiff segment may be at least partially of a radiopaque material. The stiff segment and the soft segment may be formed separately. The stiff segment and the soft segment may be bonded together. The stiff segment may be located distally of the soft segment. The segment may extend around the full circumference of the tip. The segment may be substantially tubular.

The invention also provides in another aspect a retrieval catheter tip comprising:—
an expandable tip body into which an object may be retrieved; and
at least one reinforcement element for reinforcing the tip body;
the tip body comprising a first segment along the at least one reinforcement element and a second segment distally of the at least one reinforcement element;
Area 1 being less than Area 2;
   where: Area 1 is the area of tip body material at a cross-section in the first segment; and
   Area 2 is the area of tip body material at a cross-section in the second segment.

In one embodiment of the invention the tip body has a reception space into which an object may be retrieved. The radial dimension of the reception space may vary along at least part of the length of the reception space. At least part of the reception space may taper distally from a larger radial dimension to a smaller radial dimension. The wall thickness of the tip body may vary along at least part of the length of the tip body. At least part of the tip body may taper distally from a smaller wall thickness to a larger wall thickness. The wall thickness of the tip body may be constant along at least part of the length of the tip body. The wall thickness of the tip body may be constant along the first segment and may vary along the second segment. The tip may be shaped for a smooth crossing profile. A distal end of the tip may be rounded. A distal end of the tip body may be rounded.

In a further aspect of the invention, there is provided a retrieval catheter tip suitable for exchange over a guidewire, the tip comprising:—
an expandable tip body;
the tip body having a reception space into which an object may be retrieved;
the tip body comprising a distal opening through which the object may enter the reception space;
the radial dimension of the distal opening being less than substantially three times the radial dimension of a guidewire over which the tip is exchangeable.

In one embodiment of the invention the radial dimension of the distal opening is approximately equal to two times the radial dimension of a guidewire over which the tip is exchangeable.

The radial dimension of the distal opening may be approximately 0.030″ (0.762 mm).

The radial dimension of the reception space may vary along at least part of the length of the reception space. At least part of the reception space may taper distally from a larger radial dimension to a smaller radial dimension. The tip may be shaped for a smooth crossing profile. A distal end of the tip may be rounded. A distal end of the tip body may be rounded.

According to the invention there is provided a retrieval catheter tip comprising:—
an expandable tip body into which an object may be retrieved; and
at least one reinforcement element for reinforcing the tip body.

In one embodiment of the invention the reinforcement element comprises a reinforcement column extending along at least part of the length of the tip body. The reinforcement column may extend along only part of the length of the tip body. The reinforcement column may comprise a spline.

In another embodiment the circumferential dimension of the reinforcement element varies along at least part of the length of the reinforcement element. At least part of the reinforcement element may taper distally from a larger circumferential dimension to a smaller circumferential dimension. The larger circumferential dimension may be in the range of from 120° to 170°. The smaller circumferential dimension may be in the range of from 60° to 120°. The circumferential dimension of the reinforcement element may vary along the entire length of the reinforcement element. In one case the circumferential dimension of the reinforcement element may be constant along at least part of the length of the reinforcement element. The circumferential dimension of the reinforcement element may be constant along the entire length of the reinforcement element. The circumferential dimension of the reinforcement element may be constant along a first part of the length of the reinforcement element and may vary along a second part of the length of the reinforcement element. The first part may be located proximally of the second part.

In another embodiment a distal end of the reinforcement element is rounded. The tip may comprise two reinforcement elements spaced-apart circumferentially around the tip. The reinforcement elements may diametrically oppose one another. The tip body may extend over an outer surface of the reinforcement element. The tip body may extend over an inner surface of the reinforcement element. The tip body extend between laterally adjacent reinforcement elements. At least part of the reinforcement element may be embedded within at least part of the tip body. The reinforcement element may be embedded within the tip body along only part of the length of the reinforcement element. The reinforcement element may be embedded within the tip body along substantially the full length of the reinforcement element.

In one embodiment the tip body extends distally of a distal end of the reinforcement element. The tip body may have a reception space into which an object may be retrieved. The radial dimension of the reception space may vary along at least part of the length of the reception space. At least part of the reception space may taper distally from a larger radial dimension to a smaller radial dimension. At least part of the reception space may taper distally from a smaller radial dimension to a larger radial dimension. The radial dimension of the reception space may be constant along at least part of the length of the reception space. The radial dimension of the reception space may be constant along a first part of the length of the reception space and may vary along a second part of the length of the reception space. The first part may be located proximally of the second part.

In a further embodiment the wall thickness of the tip body varies along at least part of the length of the tip body. At least part of the tip body may taper distally from a smaller wall thickness to a larger wall thickness. At least part of the tip body may taper distally from a larger wall thickness to a smaller wall thickness. The wall thickness of the tip body may be constant along at least part of the length of the tip body. The wall thickness of the tip body may be constant along a first part of the length of the tip body and may vary along a second part of the length of the tip body. The first part may be located proximally of the second part.

In another embodiment the tip is shaped for a smooth crossing profile. A distal end of the tip may be rounded. A distal end of the tip body may be rounded.

In one embodiment at least part of the tip body is coupled to at least part of the reinforcement element. At least part of the tip body may be attached to at least part of the reinforcement element. The tip body may be attached to the reinforcement element along only part of the length of the reinforcement element. The tip body may be attached to the reinforcement element along substantially the full length of the reinforcement element. The tip body may be attached to the reinforcement element around only part of the circumferential dimension of the reinforcement element. The tip body may be attached to the reinforcement element around substantially the full circumferential dimension of the reinforcement element. The tip body may be detached from the reinforcement element.

The tip body may be heat-sealed to the reinforcement element.

In a further embodiment the tip comprises a separator to separate laterally adjacent reinforcement elements. The separator may be provided on the tip body. The separator may comprise a projection projecting from a surface of the tip body. The projection may project radially inwardly from an inner surface of the tip body. A circumferential dimension of the projection may vary along at least part of the length of the projection. The projection may taper distally from a smaller circumferential dimension to a larger circumferential dimension.

In one embodiment the reinforcement element extends proximally of a proximal end of the tip body. The reinforcement element may terminate at a proximal end of the tip body. The tip may comprise an expandable member. The expandable member may extend around the reinforcement element. The expandable member may comprise a band. The expandable member may be radiopaque. The longitudinal dimension of the expandable member may be small relative to the longitudinal dimension of the tip body and/or of the reinforcement element.

The tip may comprise a limiter to limit the extent of expansion of the tip body. The limiter may be movable from a radially collapsed configuration to a radially expanded configuration. In the radially expanded configuration the limiter may be configured to resist any further expansion. The limiter may comprise a mechanical lock to resist any further expansion. The limiter may extend around the tip body. The limiter may comprise a band. The longitudinal dimension of the limiter may be small relative to the longitudinal dimension of the tip body and/or of the reinforcement element.

The invention also provides in another aspect a retrieval catheter comprising a catheter shaft and a retrieval catheter tip of the invention at a distal end of the shaft.

In one embodiment the tip is fixed to the distal end of the shaft. The reinforcement element may be fixed to the distal end of the shaft. The reinforcement element may be formed integrally with the shaft. In one case the proximal end of the tip body is spaced distally of the distal end of the catheter shaft. The outer diameter of the tip body may be substantially equal to the outer diameter of the catheter shaft.

In another aspect the invention provides a retrieval catheter tip comprising: an expandable tip body into which an object can be retrieved; and at least one reinforcement element for reinforcing the tip body; the tip comprising a soft polymer filled with radiopacifier, the tip having a material attenuation thickness coefficient (MATC) as herein defined of greater than or equal to 0.1 cm³/g.A The MACT may be greater than 0.2 cm³/g The MACT may be greater than 0.25 cm³/g The MACT may be greater than 0.3 cm³/g.

In another aspect the invention provides a retrieval catheter tip comprising: an expandable tip body into which an object can be retrieved; and at least one reinforcement element for reinforcing the tip body; the tip comprising a soft polymer filled with radiopacifier wherein the rapiopacifier is at least partially in particulate form and at least 90% of the particles have a major dimension which is less than 20 microns in length. The major dimension may be from 0.01 to 15 microns in length. The major dimension may be from 0.01 to 15 microns in length. The major dimension may be from 1 to 10 microns in length The major dimension may from 1 to 4 microns in length.

In another aspect the invention provides a retrieval catheter tip comprising: an expandable tip body into which an object can be retrieved; and at least one reinforcement element for reinforcing the tip body; the tip comprising a soft polymer filled with radiopacifier wherein the radiopaque filler is an element with an atomic number greater that 56. The radiopaque filler may be an element with an atomic number greater that 70 The radiopaque filler may be an element with an atomic number of from 73 to 83 The radiopaque filler may comprise an element belonging to periodic table group 4 to 11. The radiopaque filler may comprise an element belonging to periodic table period 4 to 6. The radiopaque filler may comprise an element belonging to periodic table group 4 to 6 and period 6 The radiopaque filler may comprise Tungsten.

In another aspect the invention provides a retrieval catheter tip comprising: an expandable tip body into which an object can be retrieved; and at least one reinforcement element for reinforcing the tip body; the tip comprising a soft polymer filled with radiopacifier wherein the secant modulus of the radiopacified tip material at 100% elongation is less than 2.0 Mpa The secant modulus of the radiopacified tip material at 100% elongation may be less than 1.5 MPa. The secant modulus of the radiopacified tip material at 100% elongation may be less than 1.1 MPa. In another aspect the invention provides a retrieval catheter tip comprising: an expandable tip body into which an object can be retrieved; and at least one reinforcement element for reinforcing the tip body; the tip comprising a soft polymer filled with radiopacifier wherein the tip material loaded with radiopacifier has an ultimate elongation of greater than 200%. The tip material loaded with radiopacifier may have an ultimate elongation of greater than 400%. The tip material loaded with radiopacifier may have an ultimate elongation of greater than 600%.

In another aspect the invention provides a retrieval catheter tip comprising: an expandable tip body into which an object can be retrieved; and at least one reinforcement element for reinforcing the tip body; the tip comprising a soft polymer filled with radiopacifier wherein the radiopacifier is present in an amount of greater than 40% of the polymer material. The radiopacifier may be present in an amount of greater than 55% of the polymer material. The radiopacifier may be present in an amount of greater than 70% of the polymer material. The radiopacifier may be present in an amount of greater than 80% of the polymer material.

### BRIEF DESCRIPTION OF THE DRAWINGS

The invention will be more clearly understood from the following description of some embodiments thereof, given by way of example only, with reference to the accompanying drawings, in which:—

FIGS. 1, 1(a), 1(b), 2, 2(a) are perspective views of a distal end of a retrieval catheter according to the invention;

FIG. 3 is a perspective view of a distal end of a catheter shaft and two reinforcement elements of the catheter of FIGS. 1, 1(a), 1(b), 2 and 2(a);

FIG. 4 is a developed plan view of the distal end of the catheter shaft and the reinforcement elements of FIG. 3;

FIG. 5 is an enlarged, developed plan view of detail C in FIG. 4;

FIG. 6 is an enlarged, developed plan view of detail D in FIG. 4;

FIG. 7 is a view along line H-H in FIG. 4;

FIGS. 8 and 9(a)(i) are cross-sectional, side views of a tip body of the catheter of FIGS. 1, 1(a), 1(b), 2 and 2(a);

FIGS. 9 and 9(a)(ii) are enlarged, cross-sectional, side views of the tip body of FIGS. 8 and 9(a)(i);

FIGS. 9(a), 10 and 11 are perspective views illustrating manufacture of the catheter of FIGS. 1, 1(a), 1(b), 2 and 2(a);

FIG. 11(a) is a cross-sectional, side view of the catheter of FIGS. 1, 1(a), 1(b), 2 and 2(a);

FIG. 11(b) is a side view of an embolic protection filter;

FIG. 11(c) is a partially cross-sectional, side view of the filter of FIG. 11(b) collapsed and retrieved into the catheter of FIG. 11(a);

FIG. 11(d) is a cross-sectional, side view of the catheter of FIG. 11(c);

FIG. 11(e) is a side view of the collapsed filter of FIG. 11(c);

FIG. 11(f) is a side view of an embolic protection filter and the catheter of FIGS. 1, 1(a), 1(b), 2 and 2(a);

FIG. 11(g) is a side view of the filter of FIG. 11(f) collapsed and retrieved in the catheter of FIG. 11(f); FIGS. 11(h) and 11(i) are cross-sectional, side views of other retrieval catheters according to the invention;

FIG. 12 is a cross-sectional, side view of a distal end of another retrieval catheter according to the invention;

FIG. 12(a) is a view similar to FIG. 12 of another retrieval catheter according to the invention;

FIG. 13 is a developed plan view of a distal end of a catheter shaft and two reinforcement elements of the catheter of FIG. 12;

FIG. 14 is a side view of a distal end of another retrieval catheter according to the invention;

FIG. 15 is a cross-sectional, side view of a distal end of a further retrieval catheter according to the invention;

FIG. 16 is a side view of a distal end of a catheter shaft and two reinforcement elements of the catheter of FIG. 15;

FIG. 17 is a side view of a distal end of a catheter shaft and two reinforcement elements of another retrieval catheter according to the invention;

FIG. 17(a) is a perspective view of a distal end of the catheter of FIG. 17;

FIG. 17(b) is a perspective view illustrating manufacture of the catheter of FIG. 17(a);

FIG. 17(c) is an enlarged, partially cut-away, perspective view of a distal end of the catheter of FIG. 17(a);

FIG. 17(d) is a cross-sectional, plan view of the catheter of FIG. 17(a);

FIG. 18 is a side view of a distal end of a catheter shaft and two reinforcement elements of another retrieval catheter according to the invention;

FIGS. 18(a) and 18(b) are perspective views of a distal end of a catheter shaft and two reinforcement elements of other retrieval catheters according to the invention;

FIGS. 19 and 19(a) are cross-sectional, side views of a distal end of a tip body of another retrieval catheter according to the invention;

FIGS. 20 to 22 are side views of a part of a tip body of further retrieval catheters according to the invention;

FIGS. 23 to 25 are cross-sectional, side views illustrating manufacture of another retrieval catheter according to the invention;

FIGS. 26 to 28 are partially cut-away, perspective views of a distal end of another retrieval catheter according to the invention;

FIG. 29 is a partially cut-away, perspective view of a tip body of the catheter of FIGS. 26 to 28;

FIG. 30 is a perspective view of a distal end of another retrieval catheter according to the invention;

FIGS. 31 and 32 are partially cut-away, perspective views of a distal end of the catheter of FIG. 30;

FIG. 33 is a perspective view illustrating manufacture of the catheter of FIG. 30;

FIGS. 34 and 35 are perspective views of a distal end of further retrieval catheters according to the invention;

FIG. 36 is a perspective view illustrating manufacture of the catheter of FIG. 35;

FIGS. 37 and 38 are perspective views of a distal end of another retrieval catheter according to the invention;

FIGS. 39 to 42 are further views of the catheter of FIGS. 37 and 38;

FIGS. 43 and 44 are partially cross-sectional, side views of another retrieval catheter according to the invention retrieving an embolic protection filter;

FIGS. 45 and 46 are views similar to FIGS. 43 and 44 of another retrieval catheter according to the invention;

FIGS. 47 and 48 are views similar to FIGS. 43 and 44 of a further retrieval catheter according to the invention;

FIG. 49 is a cross-sectional, side view of another retrieval catheter according to the invention;

FIG. 50 is a cross-sectional, plan view of the catheter of FIG. 49;

FIG. 51 is a view along line D-D in FIG. 49;

FIG. 52 is a view along line F-F in FIG. 49;

FIG. 53 is a view along line E-E in FIG. 49;

FIGS. 54 to 58 are views similar to FIGS. 49 to 53 of another retrieval catheter according to the invention;

FIGS. 59 to 63 are views similar to FIGS. 49 to 53 of a further retrieval catheter according to the invention;

FIG. 64 is a cross-sectional, side view of another retrieval catheter according to the invention;

FIG. 65 is a view along line D-D in FIG. 64;

FIG. 66 is a view along line F-F in FIG. 64;

FIG. 67 is a view along line E-E in FIG. 64;

FIGS. 68 to 83 are views similar to FIGS. 64 to 67 of other retrieval catheters according to the invention;

FIG. 84 is a partially cut-away, perspective view of a distal end of another retrieval catheter according to the invention;

FIG. 85 is a cross-sectional, side view of the catheter of FIG. 84;

FIG. 86 is a side view of the catheter of FIG. 84;

FIGS. 87 and 88 are perspective views of segments of a tip body of the catheter of FIG. 84;

FIGS. 89 to 94 are perspective views illustrating manufacture of the catheter of FIG. 84;

FIG. 95 is a plan view of the distal end of the catheter of FIG. 84 mounted in a tray;

FIG. 96 is a diagram illustrating the possible outcomes for an x-ray incident on a single section of material;

FIG. 97 illustrates that in interventional x-ray imaging an x-ray beam may pass through a number of materials;

FIG. 98 is an example of the attenuation profile for elemental Tungsten;

FIG. 99 is an example of the attenuation profile for human bone;

FIGS. 100 to 102 are various views of a retrieval catheter tip of the invention;

FIG. 103 illustrates stress strain curves for the polymer SEBS 45A without radiopaque loading and with a loading of 70% radiopacifier; and

FIGS. 103 and 104 are schematic cross sectional views of retrieval catheter tips of the invention.

### DETAILED DESCRIPTION

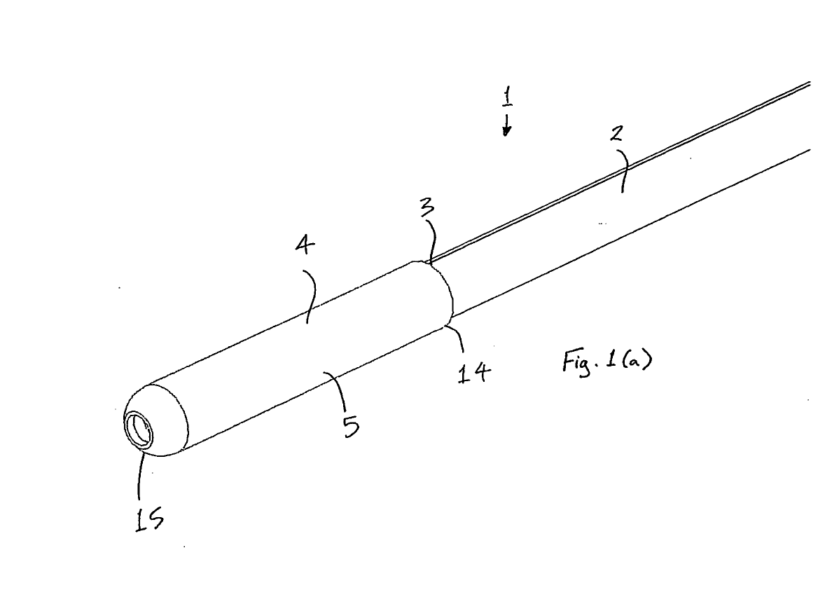

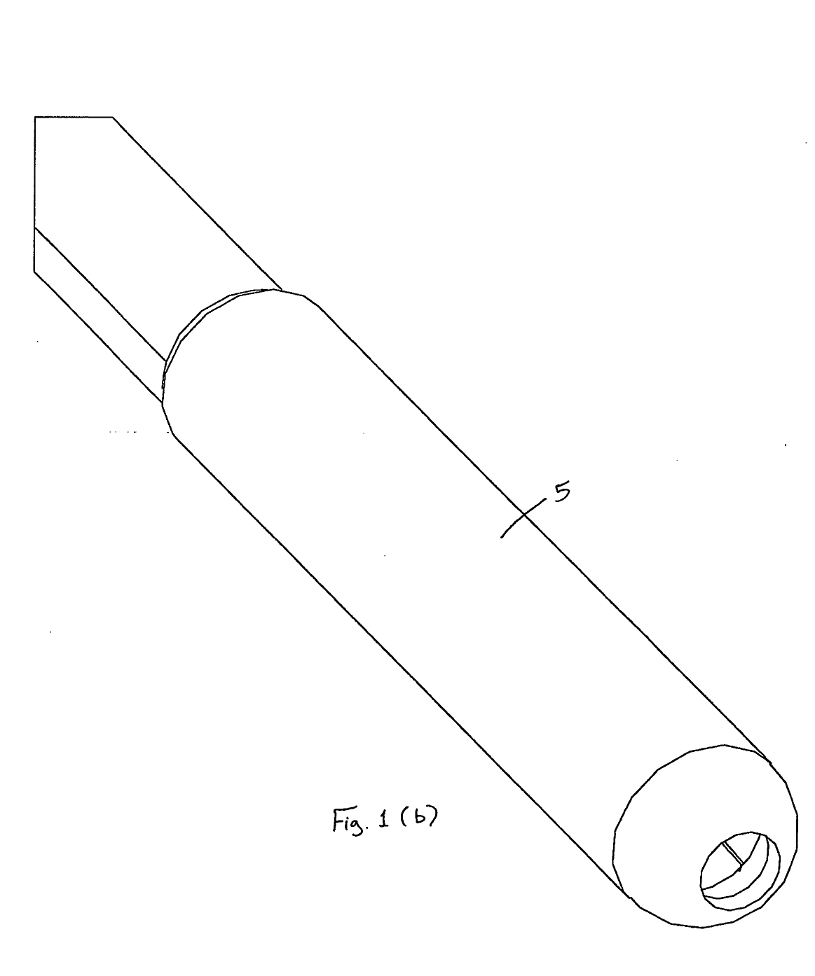

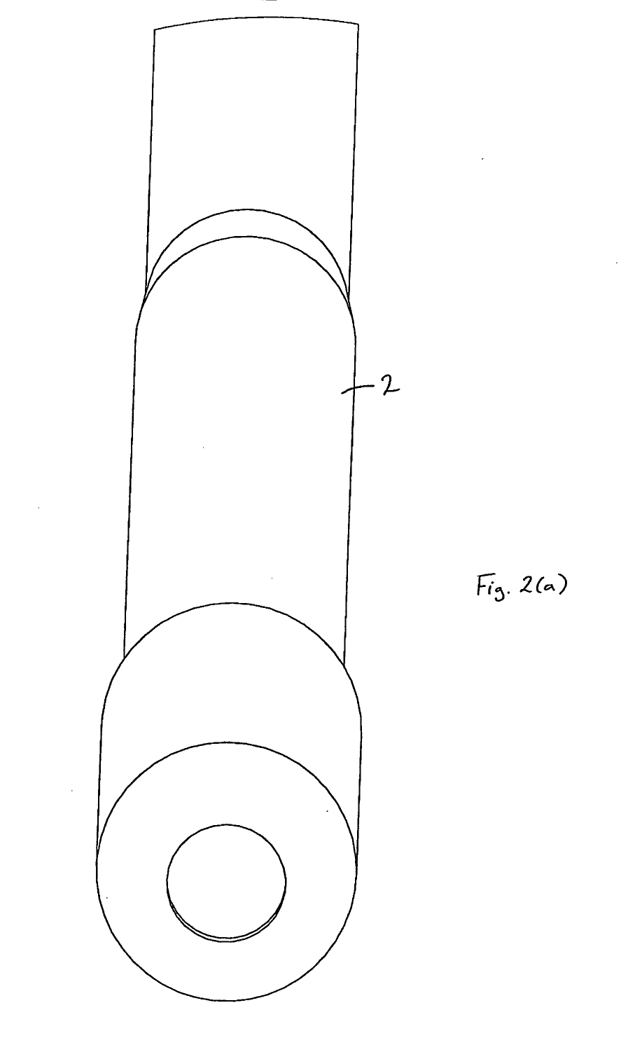

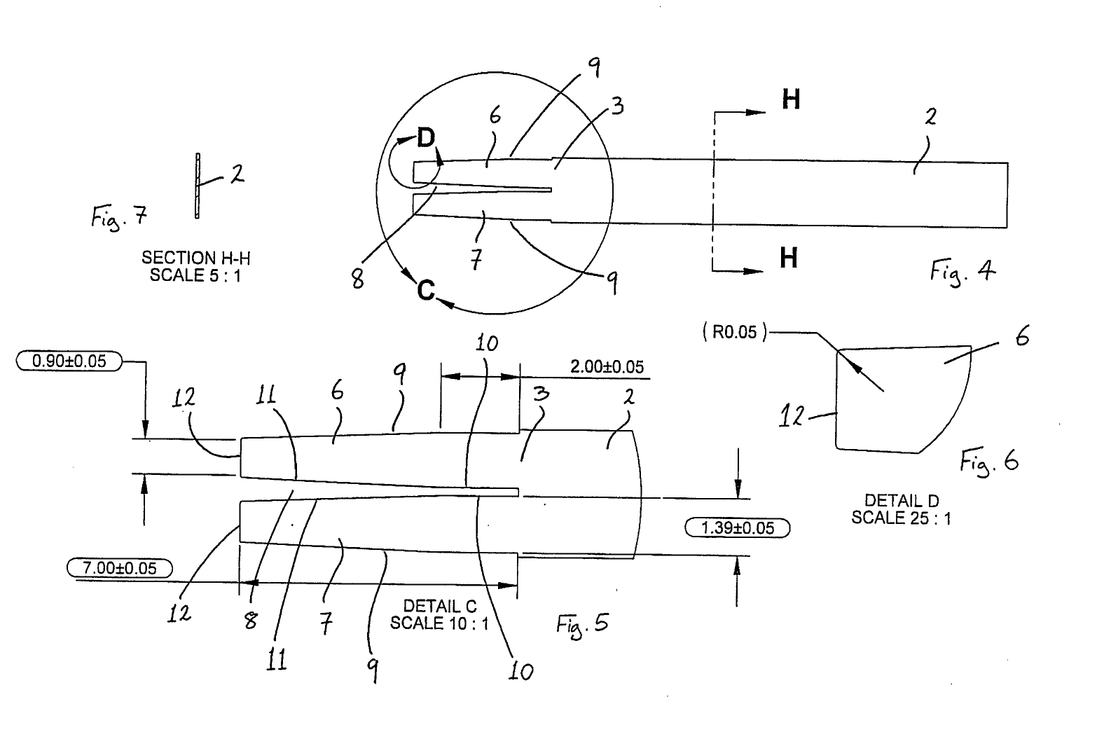

Referring to the drawings, and initially to FIGS. 1 to 11 thereof, there is illustrated a retrieval catheter 1 according to the invention. The catheter 1 comprises a catheter shaft 2 having a guidewire lumen extending therethrough from a proximal end of the shaft 2 to a distal end 3 of the shaft 2, and a retrieval catheter tip 4 at the distal end 3 of the shaft 2.

The tip 4 comprises an expandable tip body 5 and two reinforcement columns 6, 7 extending along part of the length of the tip body 5. In this case, each reinforcement column 6, 7 is provided in the form of a reinforcement spline.

The two reinforcement columns 6, 7 are spaced-apart circumferentially around the tip 4, and in this case are located diametrically opposing one another.

The reinforcement columns 6, 7 are embedded within the tip body 5, with the tip body 5 extending over the entire outer surface of the reinforcement columns 6, 7 and over the entire inner surface of the reinforcement columns 6, 7.

The tip body 5 may also extend within the longitudinal space between laterally adjacent columns 6, 7.

The tip body 5 is fixedly attached to the reinforcement columns 6, 7, in this case by heat-sealing.

The distal end 15 of the tip body 5 extends distally of the distal end 12 of the reinforcement columns 6, 7, and the distal end 15 of the tip body 5 is rounded. In this manner, the tip 4 presents a smooth crossing profile to facilitate passage of the catheter 1 through narrow and/or tortuous body passages, and to minimise the discomfort and/or damage caused during advancement of the catheter 1 through the body passageway.

The tip body 5 has a reception space 13 extending therethrough from a proximal end 14 of the tip body 5 to a distal end 15 of the tip body 5. The reception space 13 facilitates retrieval of an object, such as an embolic protection filter, into the tip body 5.

The reinforcement columns 6, 7 are stiff relative to the tip body 5 and act to reinforce the tip body 5, especially during retrieval of an object into the reception space 13, to minimise the possibility of buckling or collapse of the tip 4.

It has been found that the use of two reinforcement columns 6, 7 optimises the balance between achieving buckle strength to prevent buckling of the tip 4 during retrieval, while maintaining sufficient flexibility to enable passage of the tip 4 through potentially narrow and/or tortuous body passageways.

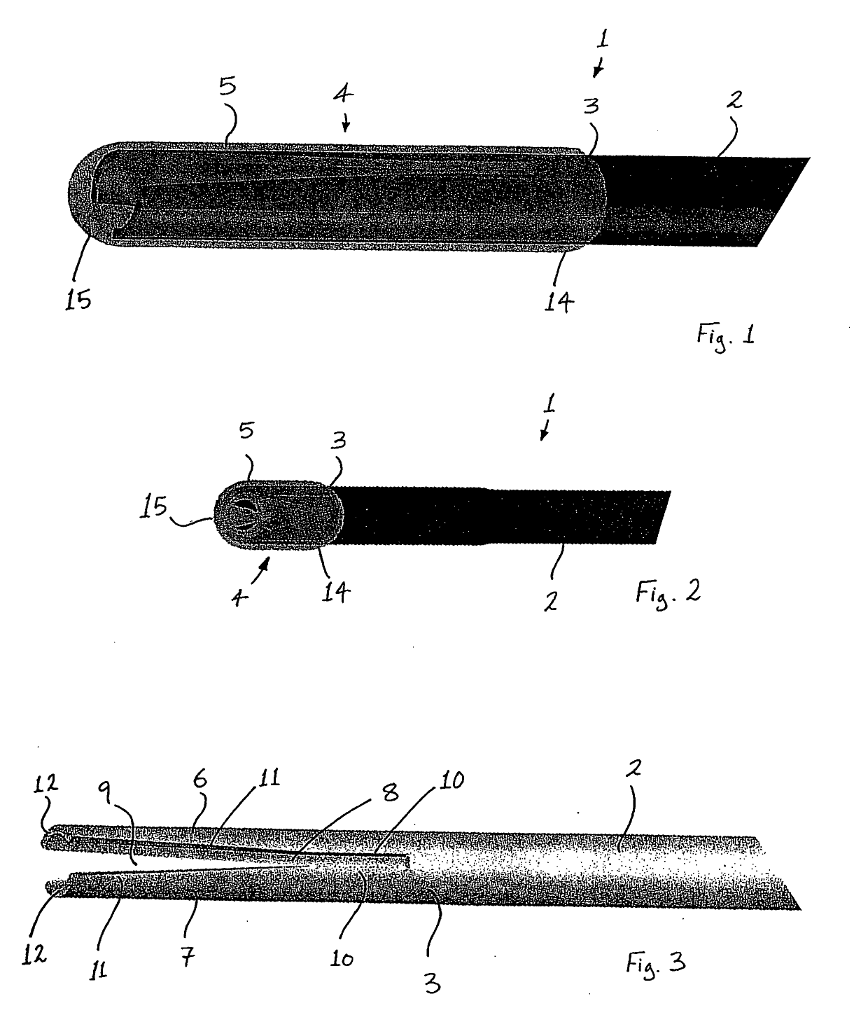

FIG. 2(a) illustrates the distal catheter shaft 2 flaring up to provide the reception space for a retrieved load.

The tip body 5 may be loaded with a radiopaque filler, such as Tungsten, to enhance visibility of the tip 4.

FIG. 1(b) illustrates the single c-flex layer expansive tip 5 with radiopaque filler.

The radiopaque filler may be provided at the distal region of the tip body 5 only. The distal region may extend for a length of for example 2-3 mm. The proximal portion of the tip body 5 may remain free from radiopaque filler. In this case the proximal portion would retain a higher tear strength than the distal portion.

Each reinforcement column 6, 7 is arcuate in cross-sectional end view. The circumferential dimension of each arcuate reinforcement column 6, 7 is constant along a proximal part 10 of the reinforcement column 6, 7, and varies along a distal part 11 of the reinforcement column 6, 7. Along the distal part 11, each reinforcement column 6, 7 tapers distally from a relatively large circumferential dimension at the proximal part 10 to a relatively small circumferential dimension at the distal end 12 of the reinforcement column 6, 7. In this case the circumferential dimension at the proximal part 10 is in the range of from 120° to 170°, and the circumferential dimension at the distal end 12 is in the range of from 60° to 120°.

The change in circumferential dimension of the columns 6, 7 provides a gradual increase in radial stiffness from the distal end 15 of the tip body 5 towards the distal end 3 of the shaft 2.

The variation in circumferential dimension of the columns 6, 7 also creates a lead-in section for ease of retrieval of an embolic protection filter into the tip body 5.

The tip body 5 has a main part 16 and a distal nose part 17 at the distal end 15. The main part 16 extends from the proximal end 14 to the distal nose part 17. Along the main part 16, the wall thickness of the tip body 5 is constant and the diameter of the reception space 13 is constant. Along the distal nose part 17, the wall thickness of the tip body 5 varies along the length of the distal nose part 17. In particular the wall thickness of the tip body 5 tapers distally from the relatively small wall thickness at the main part 16 to a relatively large wall thickness at the distal end 15. Similarly, along the distal nose part 17, the diameter of the reception space 13 varies along the length of the distal nose part 17. In particular the diameter of the reception space 13 tapers distally from the relatively large diameter at the main part 16 to a relatively small diameter at the distal end 15. This variation in the wall thickness and the diameter assists in preventing “fish-mouthing” of the open mouth at the distal end 15 of the tip body 5.

Typical wall thicknesses of the tip body 5 are from 0.004″ to 0.006″.

The distal nose part 17 tapers distally inwardly towards a guidewire over which the retrieval catheter may be passed. By tapering inwardly towards the guidewire, the nose part 17 assists in keeping the catheter centred on the guidewire. This arrangement results in a retrieval catheter which is exchangeable over a guidewire in a centred manner without an additional centreing catheter or centreing element being required.

FIGS. 9(a)(i) and 9(a)(ii) illustrate that the nose 17 may be circular or parabolic. The extra material in the nose 17 allows the tip nose 17 to expand more than twice its original size.

Because there is no separate centreing catheter or centreing element, it is not necessary to move such an element relative to the tip 4 to retrieve the filter into the tip 4.

The reinforcement columns 6, 7 are fixedly attached to the distal end 3 of the catheter shaft 2. In particular the reinforcement columns 6, 7 are formed integrally with the catheter shaft 2, in this case, by laser-machining two slots 8, 9 from a unitary tubular shaft. In this manner, the tip 4 is fixedly attached to the distal end 3 of the catheter shaft 2.

It will be appreciated that the slots 8, 9 may be formed using a variety of different manufacturing techniques. One possible means of forming the slots 8, 9 is by cutting the slots 8, 9 into the catheter shaft 2.

Typical lengths for the slots 8, 9 are from 6 mm to 12 mm.

To manufacture the retrieval catheter 1, a unitary tubular shaft has the two slots 8, 9 laser machined in an end of the shaft. In this manner, the catheter shaft 2 and the two reinforcement columns 6, 7 are formed. The slots 8, 9 are machined in a manner suitable to form the reinforcement columns 6, 7 in the configuration described above.

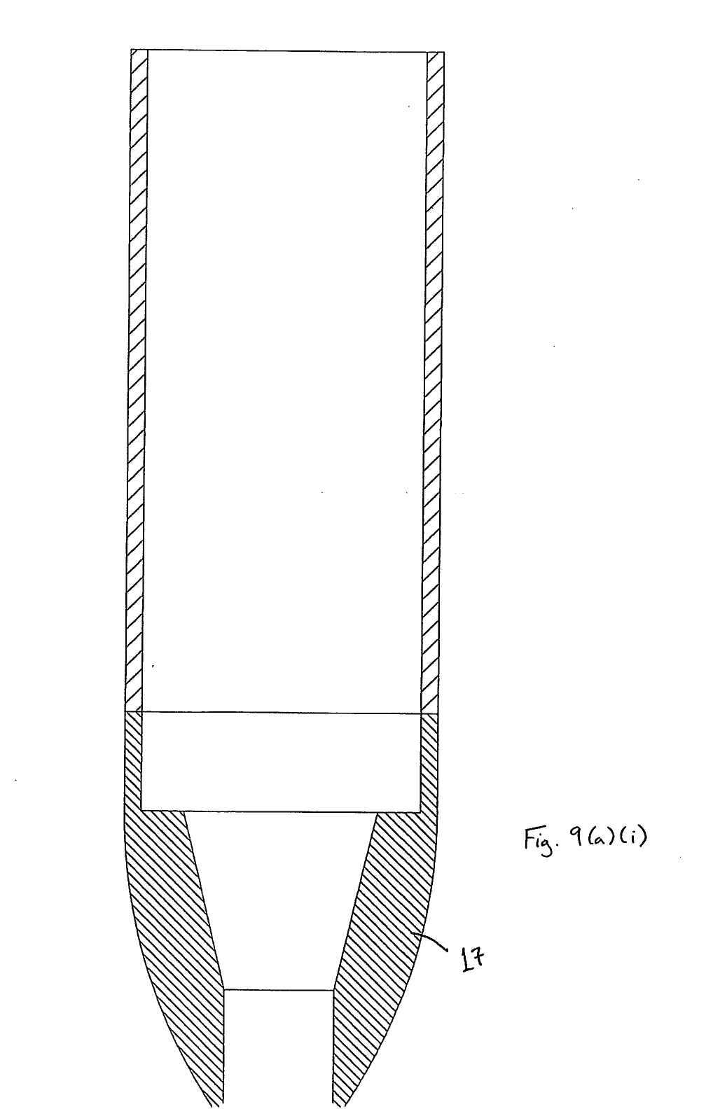

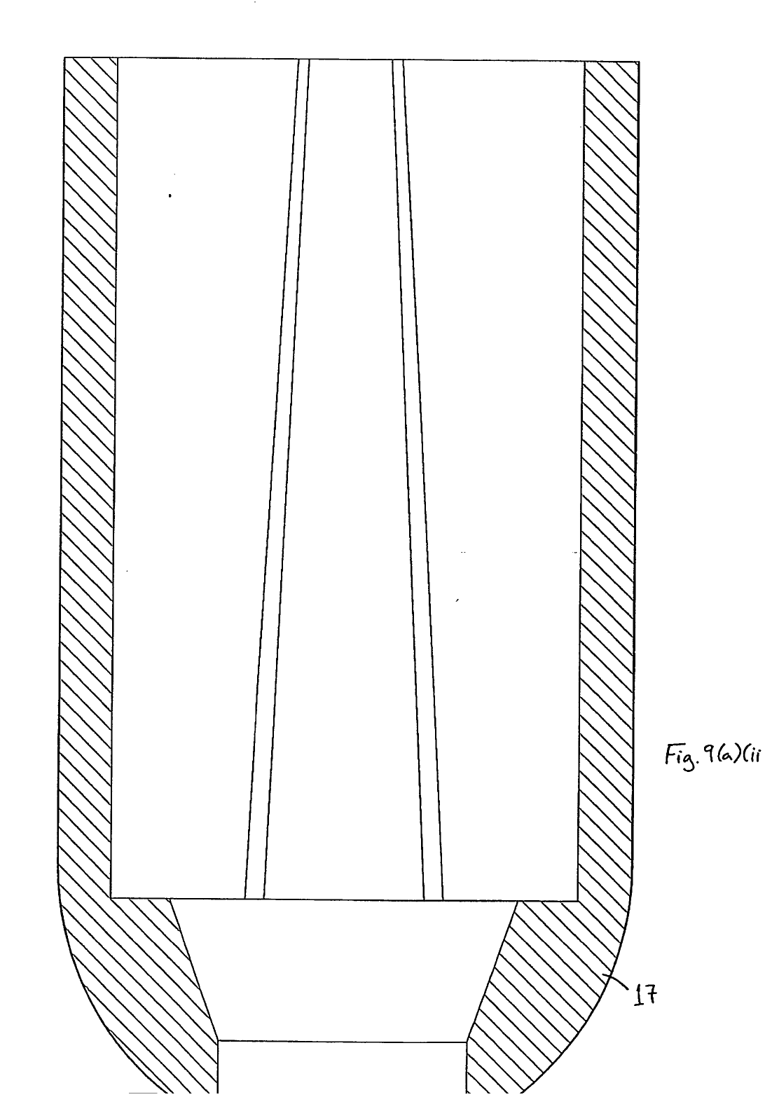

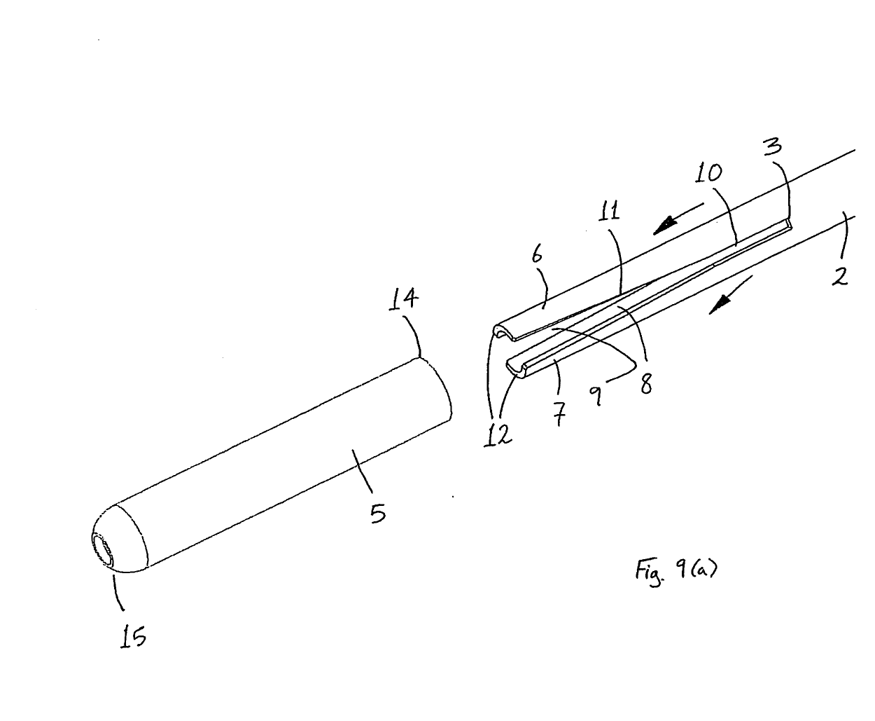

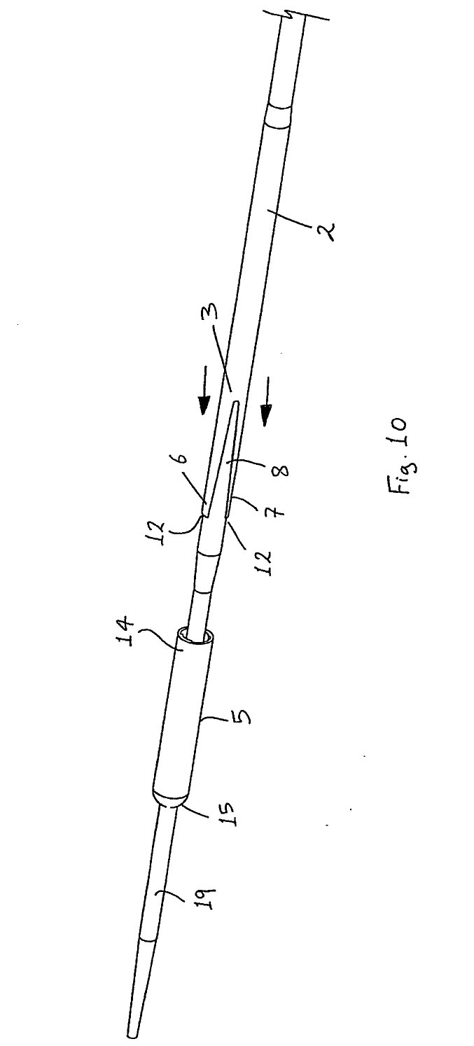

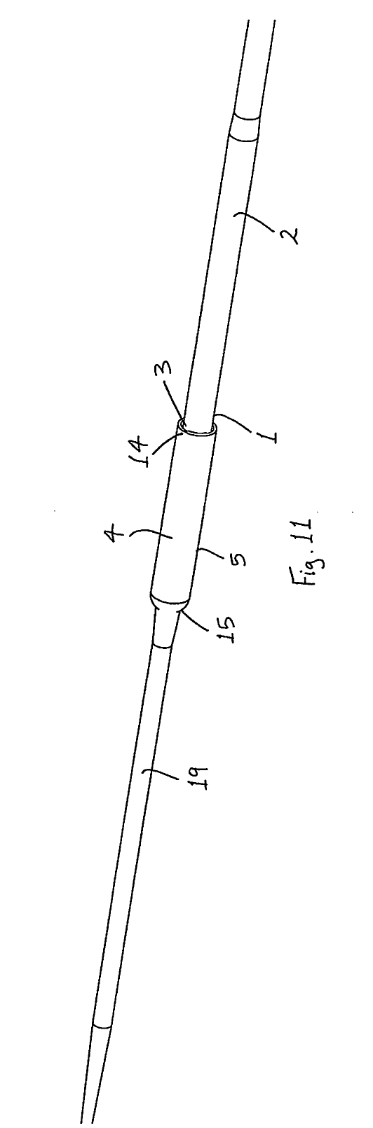

The tip body 5 is then mated with the catheter shaft 2, as illustrated in FIG. 9(a). In one case, the tip body 5 is positioned around a mandrel 19, and the catheter shaft 2 is slid over the mandrel 19 to embed the reinforcement columns 6, 7 into the tip body 5, as illustrated in FIGS. 10 and 11. The tip body 5 and the reinforcement columns 6, 7 are then heat-sealed together to form the tip 4.

The mandrel 19 provides a means of controlling the internal geometry of the tip body 5.

In use, the retrieval catheter 1 is inserted into a body passageway, in this case a vasculature, and advanced through the vasculature until the tip 4 is proximally adjacent of an object to be retrieved, such as an embolic protection filter 200 with captured embolic material 201 therein. The tip 4 is then moved distally relative to the filter 200 to retrieve at least part of the filter 200 with the captured embolic material 201 into the tip 4. The tip body 5 expands radially outwardly to accommodate the relatively large embolic protection filter 200 into the tip 4 during retrieval.

In one arrangement, and as illustrated in FIGS. 11(a) to 11(e), after retrieval of the filter 200 a proximal portion 203 of the filter 200 is retrieved into the catheter shaft 2, an intermediate portion of the filter 200 is retrieved into the tip 4, and a distal portion 202 of the filter 200 protrudes out of the distal end of the tip 4. A proximal part 204 of the intermediate portion of the filter 200 is retrieved into the proximal part 10 of the reinforcement columns 6, 7, while a distal part 205 of the intermediate portion of the filter 200 is retrieved into the distal part 11 of the reinforcement columns 6, 7. In one case the captured embolic load 201 is retained in the distal part 205 of the intermediate portion of the filter 200 within the distal part 11 of the reinforcement columns 6, 7.

The distal part 11 of the reinforcement columns 6, 7 acts a lead-in or guide for the filter 200 being retrieved. The proximal part 10 of the reinforcement columns 6, 7 acts a stiffness transition. The catheter shaft 2 acts as a constraint to constrain the retrieved filter.

A support frame 206 of the embolic protection filter 200 collapses down upon retrieval of the filter 200, as illustrated in FIGS. 11(c) and 11(e).

In another case the proximal part 10 of the reinforcement columns 6, 7 may act as a compaction zone for the captured embolic material 201. In particular the proximal part 10 is a uniform strain zone which thus provides constant radial force on a retrieved filter.

In another arrangement, the entire retrieved embolic protection filter 200 may be pulled proximally passed the proximal part 10 into the unslotted catheter shaft 2. The unslotted shaft 2 is a rigid, solid tube. The retrieved filter will therefore not increase the outer diameter of the unslotted catheter shaft 2, as the rigid shaft 2 will constrain the retrieved filter.

No buckling of the tip 4 occurs during retrieval due to the presence of the reinforcement columns 6, 7.

When the filter has been fully retrieved into the tip 4, the retrieval catheter 1 is withdrawn from the vasculature.

The resilient nature of the tip body 5 ensures that the embolic protection filter and the captured embolic material are safely retained with the tip 4, during retrieval and withdrawal.

The retrieval catheter 1 is suitable, in certain cases, for retrieving an embolic protection filter having an embolic load of up to 20 mg.

Typical diameters for an embolic protection filter are 2.5 mm to 7 mm.

FIGS. 11(f) and 11(g) illustrate that the distal olive 202 remains external to the retrieval catheter post retrieval. This allows a clinician to pull the filter out and examine particles.

In FIG. 11(h) a tube 110 extends from a rapid exchange port through the catheter shaft 2. This RX tunnel 110 may form a filter abutment surface to act as a stop to control the extent of retrieval of the filter.

In FIG. 11(i), the filter abutment is provided by an internal stop 111 adhesively fixed in place.

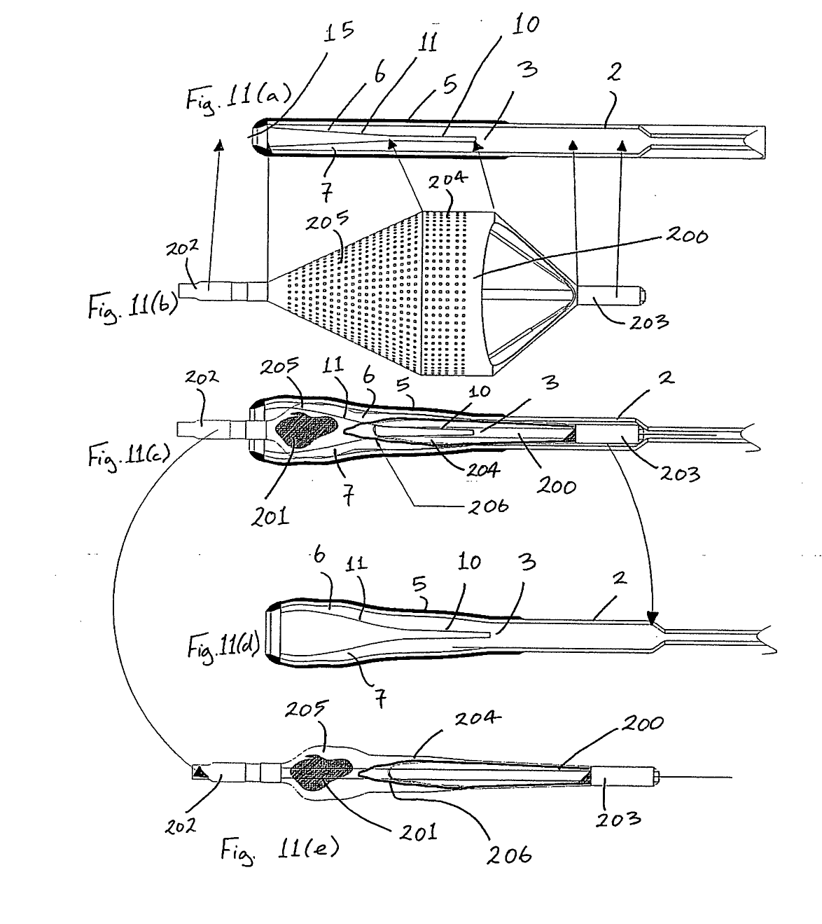

FIGS. 12 and 13 illustrate another retrieval catheter 20 according to the invention, which is similar to the catheter 1 of FIGS. 1 to 11, and similar elements in FIGS. 12 and 13 are assigned the same reference numerals.

In this case, the circumferential dimensions of each arcuate reinforcement column 21, 22 varies along the entire length of the reinforcement column 21, 22. In particular each reinforcement column 21, 22 tapers distally from a relatively large circumferential dimension at the distal end 3 of the catheter shaft 2 to a relatively small circumferential dimension at the distal end 12 of the reinforcement element 21, 22.

FIG. 12(a) illustrates the inverting tip 112 and the splines 113.

In FIG. 14 there is illustrated another retrieval catheter 25 according to the invention, which is similar to the catheter 20 of FIGS. 12 and 13, and similar elements in FIG. 14 are assigned the same reference numerals.

In this case the distal end 12 of each reinforcement column 21, 22 is rounded. In addition the region of connection 26 of each reinforcement column 21, 22 to the distal end 3 of the catheter shaft 2 is rounded. The rounded ends reduce the stress in the material transition.

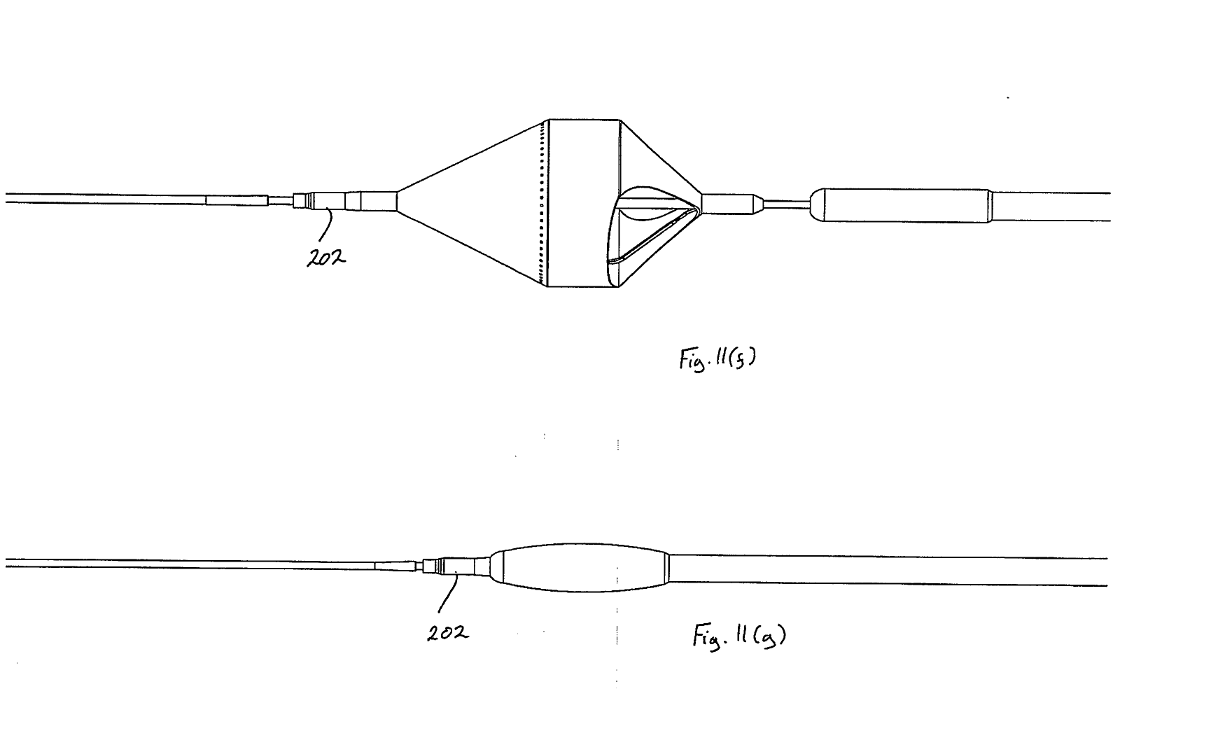

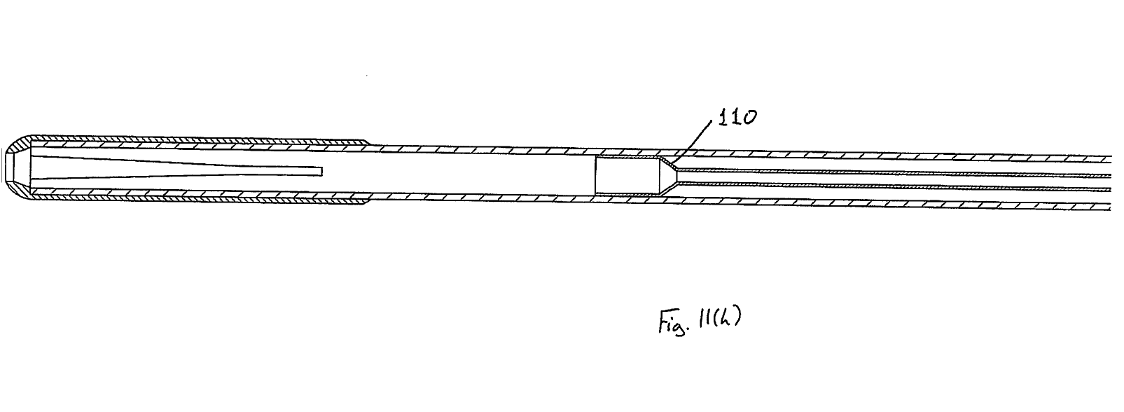

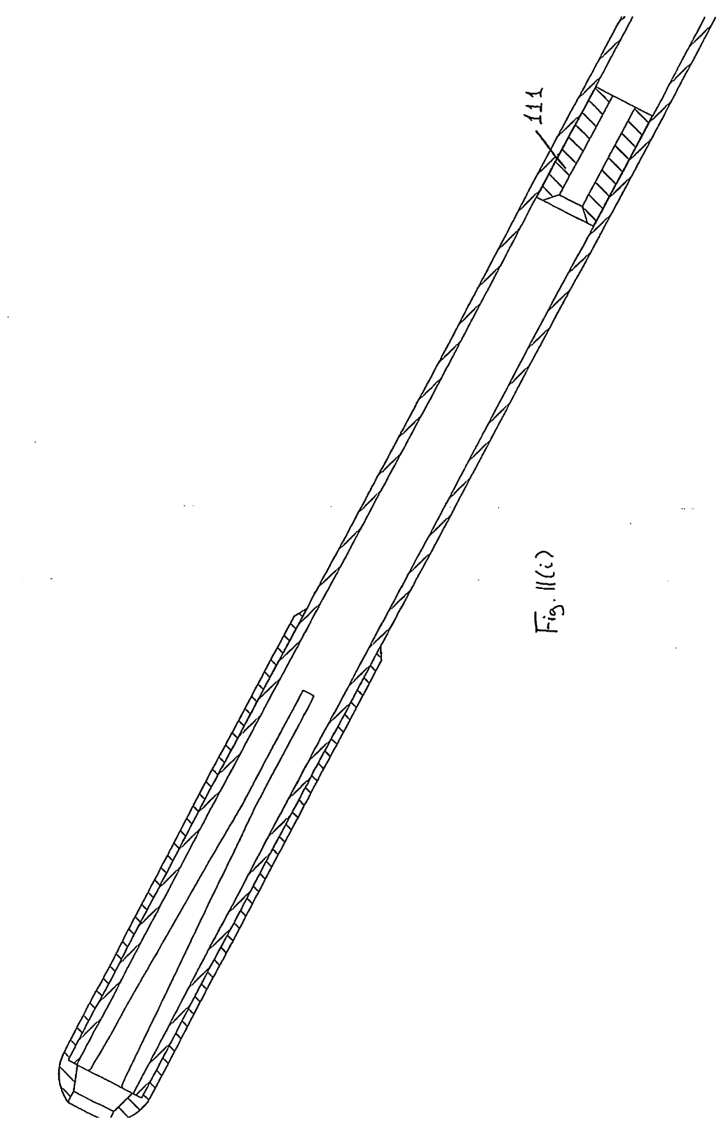

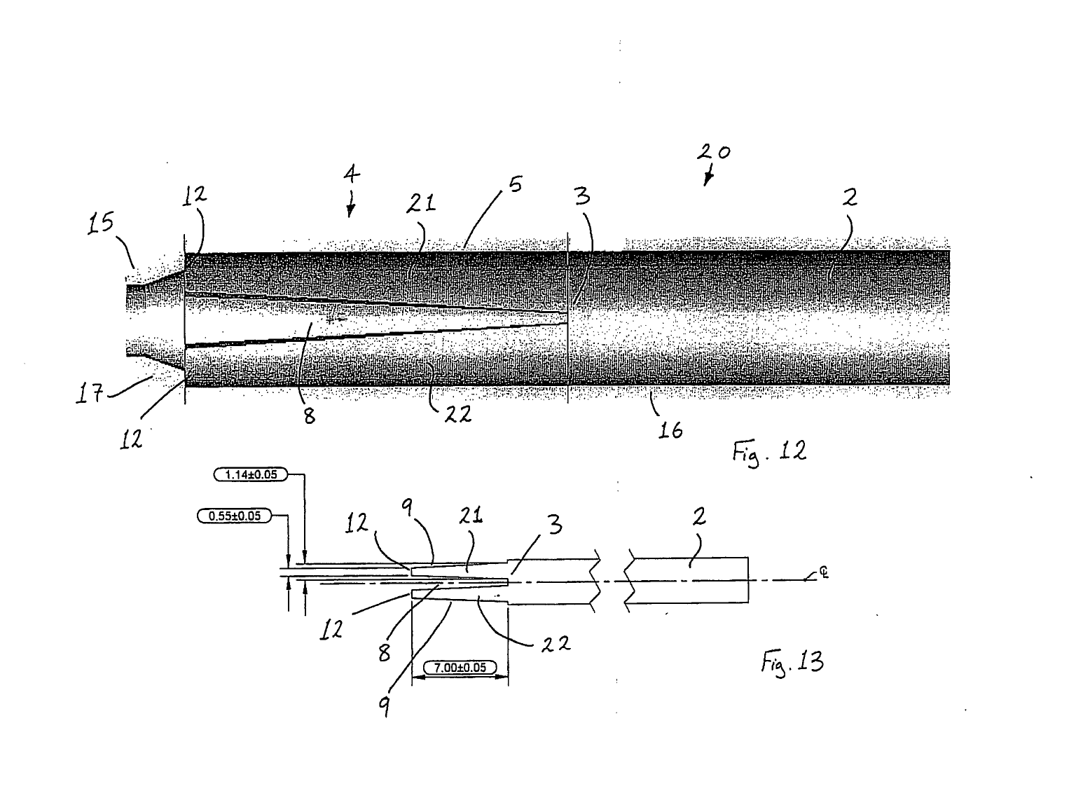

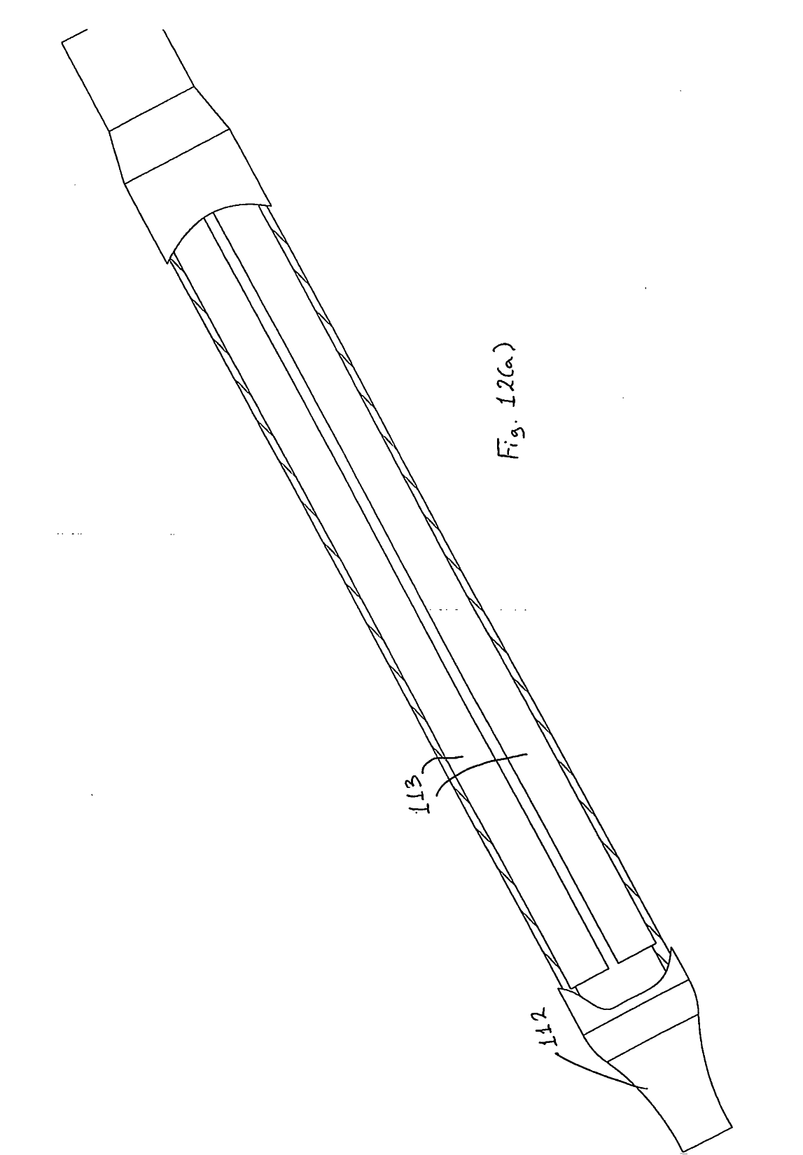

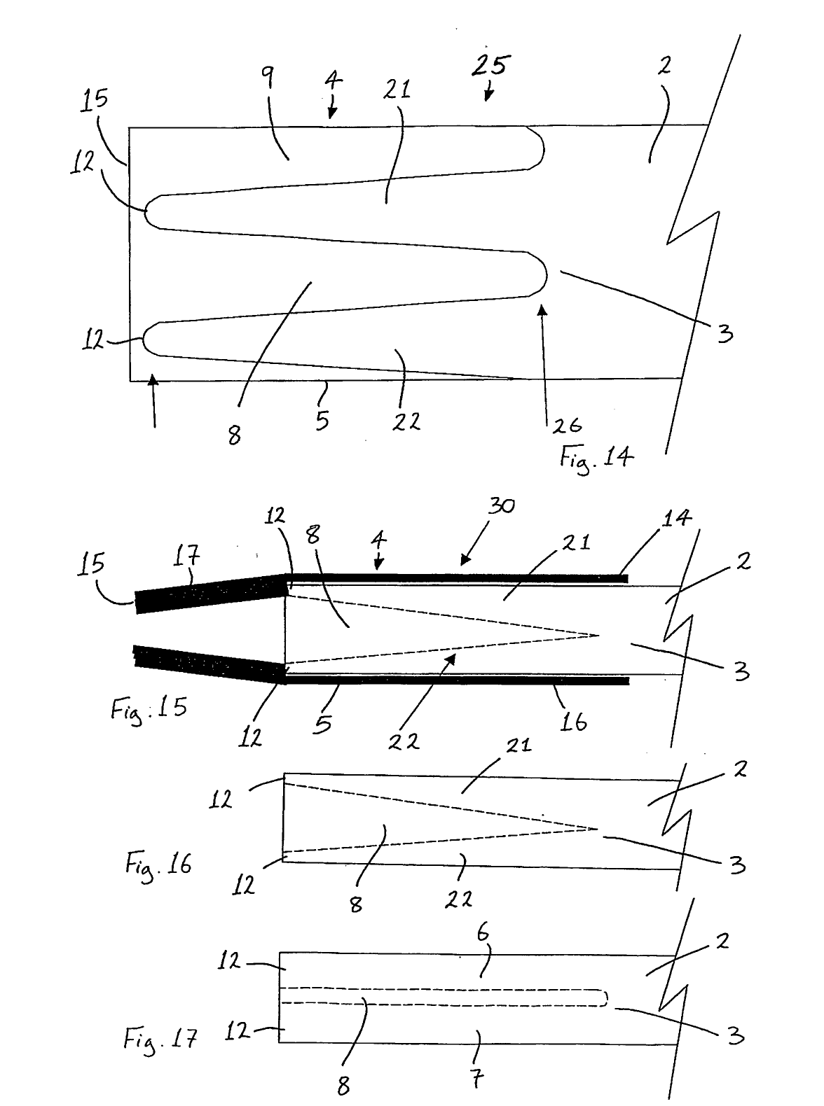

Referring to FIGS. 15 and 16, there is illustrated another retrieval catheter 30 according to the invention, which is similar to the catheter 20 of FIGS. 12 and 13, and similar elements in FIGS. 15 and 16 are assigned the same reference numerals.

In the catheter 30, there is a linear increase in radial force from the distal end 15 of the tip body 5 to the distal end 3 of the catheter shaft 2.

FIGS. 17 to 17(c) illustrate an alternative construction for the reinforcement columns 6, 7. In this case the circumferential dimension of each arcuate reinforcement column 6, 7 is constant along the entire length of the reinforcement column 6, 7. There is therefore a uniform radial force along the length of the tip body 5.

In this case the tip body 5 is provided extending along the slots 8, 9 between adjacent columns 6, 7. The tip body 5 does not extend over the outer surface or over the inner surface of the tip body 5.

As illustrated in FIG. 17(b), the tip body 5 comprises two proximally extending fingers which mate with the columns 6, 7 in a keying arrangement to form the tip 4.

The distal nose part 17 of the tip body 5 extends distally beyond the columns 6, 7, and acts as a flaring element/lead-in element.

It will be appreciated that the tip body 5 may be attached to the columns 6, 7 and/or the catheter shaft 2 in any suitable manner. In certain cases, the tip body 5 may be formed integrally with the columns 6, 7 and/or catheter shaft 2.

The retrieval catheter of the invention is capable of crossing a narrowed site in a vasculature, such as a region of stenosis or a deployed stent, while retaining the capability to retrieve an object, such as an embolic protection filter, downstream of the narrowed site.

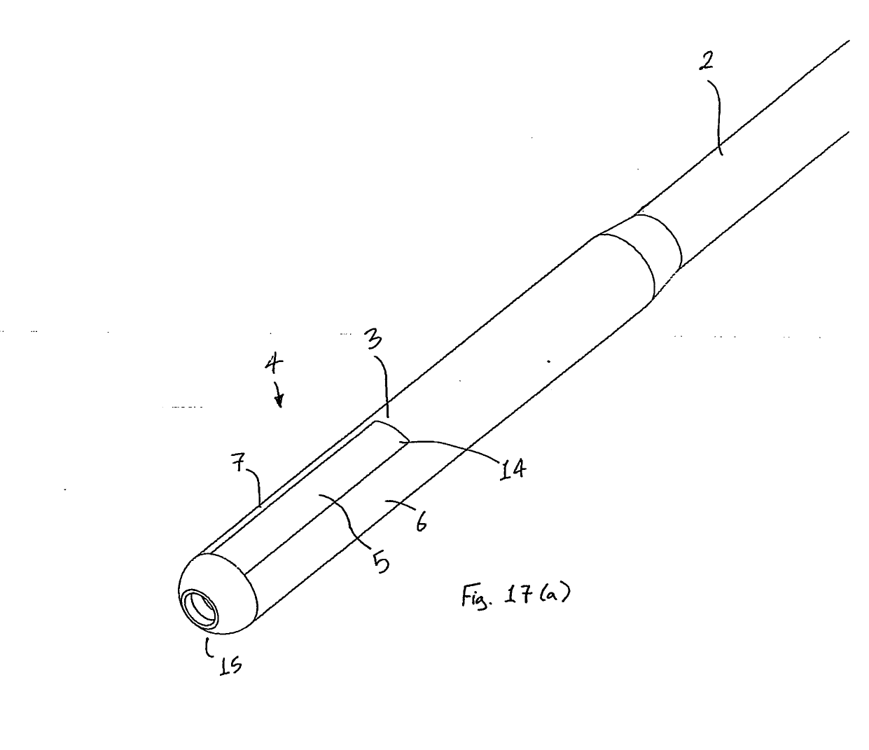

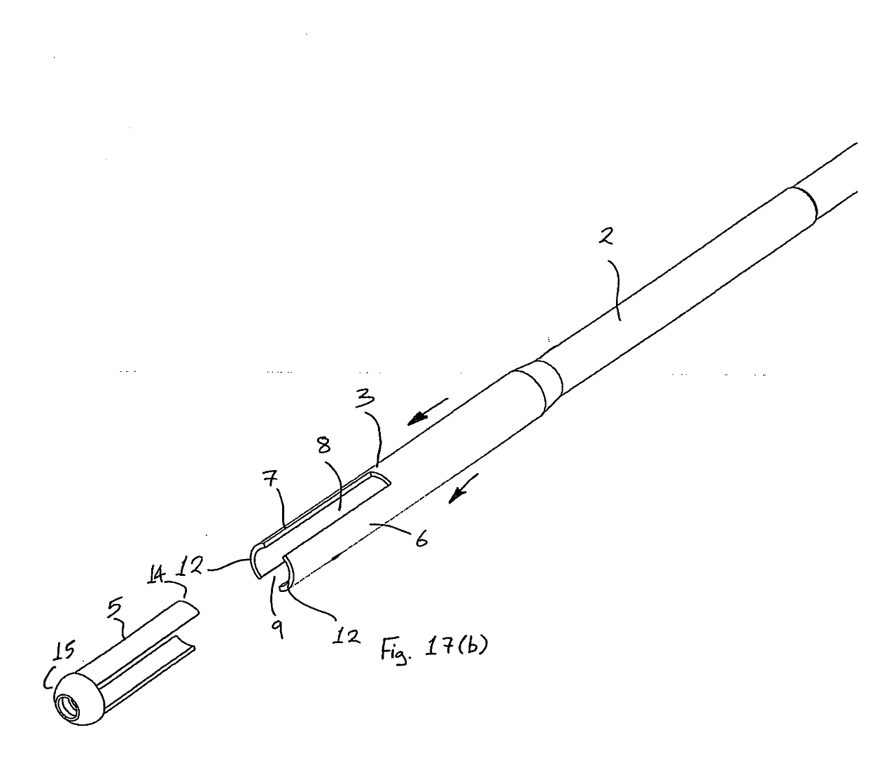

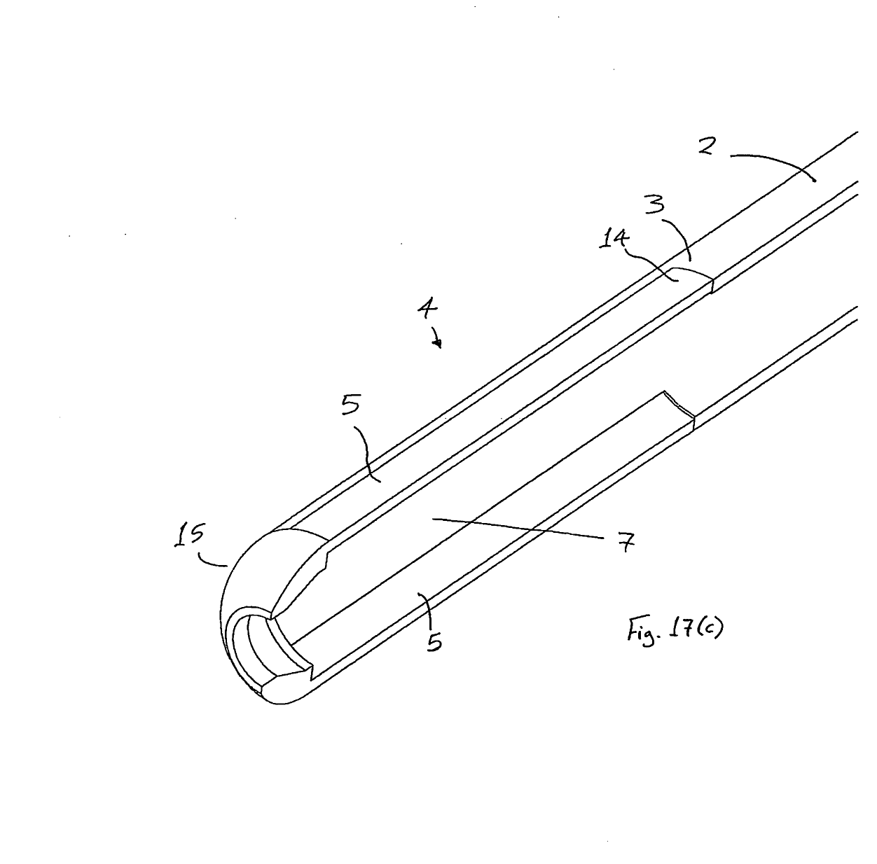

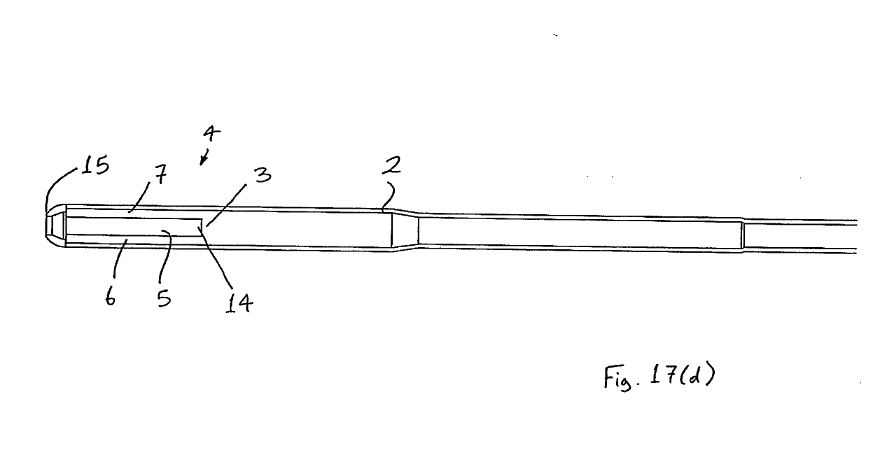

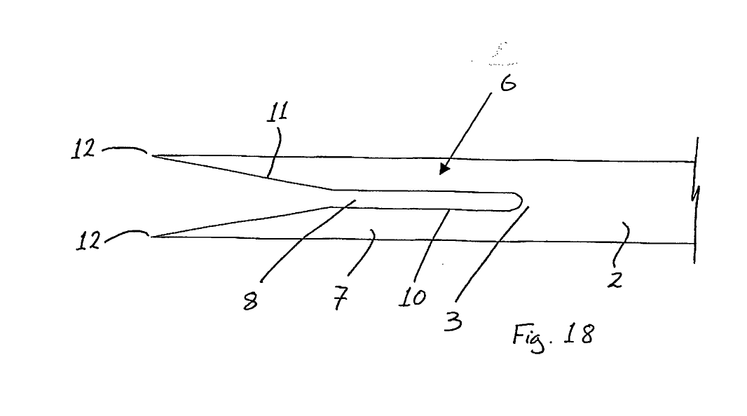

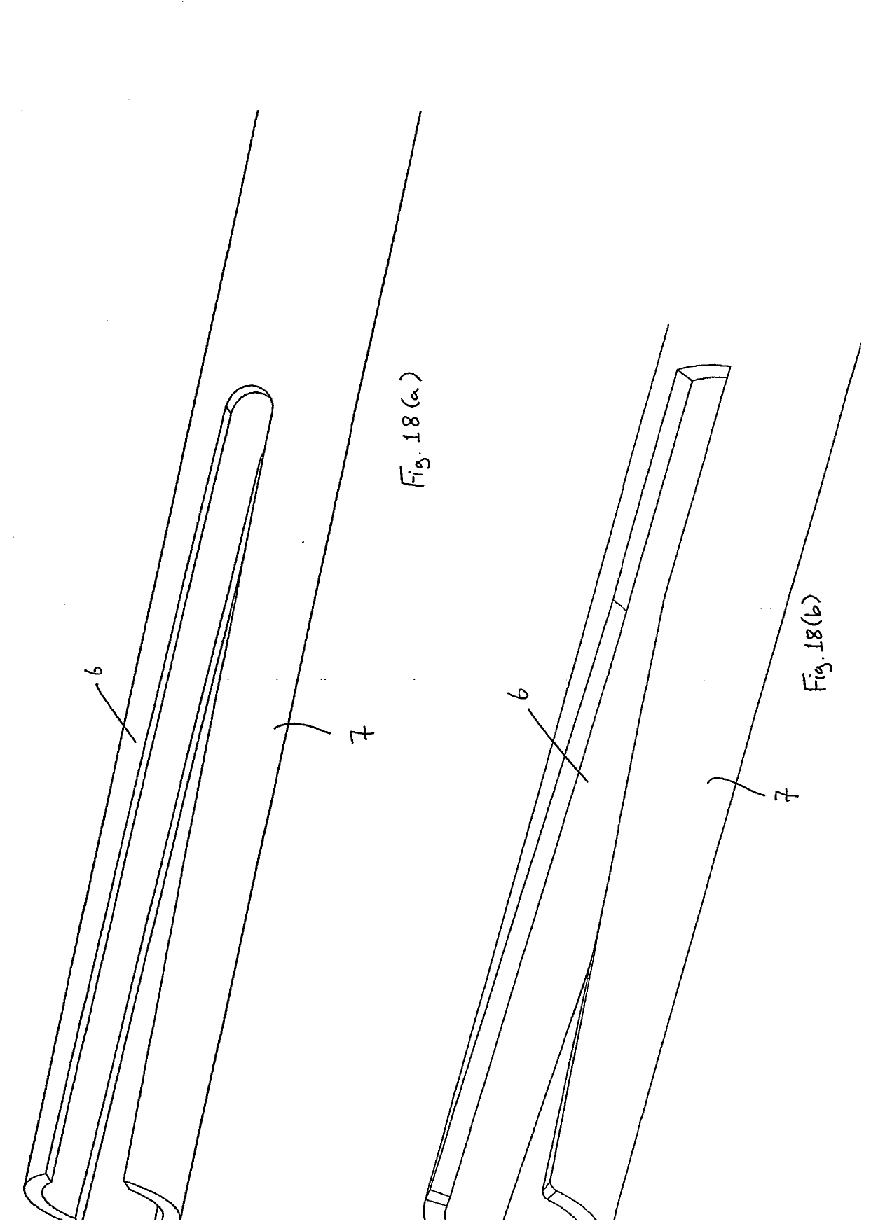

The retrieval catheter of FIG. 18 employs the linear increase in radial force along a distal region of the tip 4, as discussed in relation to FIGS. 15 and 16 above, and also enjoys the uniform radial force along a proximal region of the tip 4, as discussed above in relation to FIG. 17.

In FIG. 18(a) a rounded edge is provided at the proximal connection of the two reinforcement elements 6, 7.

In FIG. 18(b), a sharp edge is provided at the proximal connection of the two reinforcement elements 6, 7. The sharp edge acts a stress raiser.

FIGS. 19 to 22 illustrate various alternative constructions for the tip body 5. The tip body 5 of FIG. 19 is similar to the tip body 5 described previously with reference to FIG. 9.

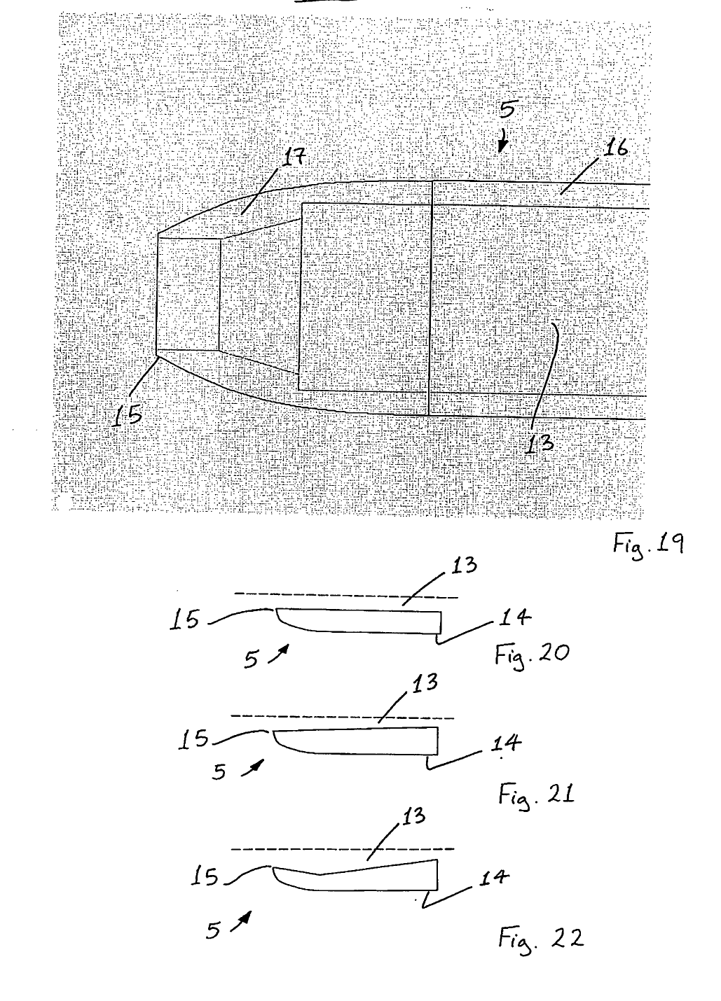

In FIG. 20, the wall thickness of the tip body 5 and the diameter of the reception space 13 are constant along the entire length of the tip body 5.

In FIG. 21, the wall thickness of the tip body 5 tapers distally along the entire length of the tip body 5 from a relatively large wall thickness at the proximal end 14 to a relatively small wall thickness at the distal end 15. Similarly the diameter of the reception space 13 tapers distally along the entire length of the tip body 5 from a relatively small diameter at the proximal end 14 to a relatively large diameter at the distal end 15.

FIG. 22 illustrates a further embodiment of the tip body 5 having an alternative variation in wall thickness and diameter.

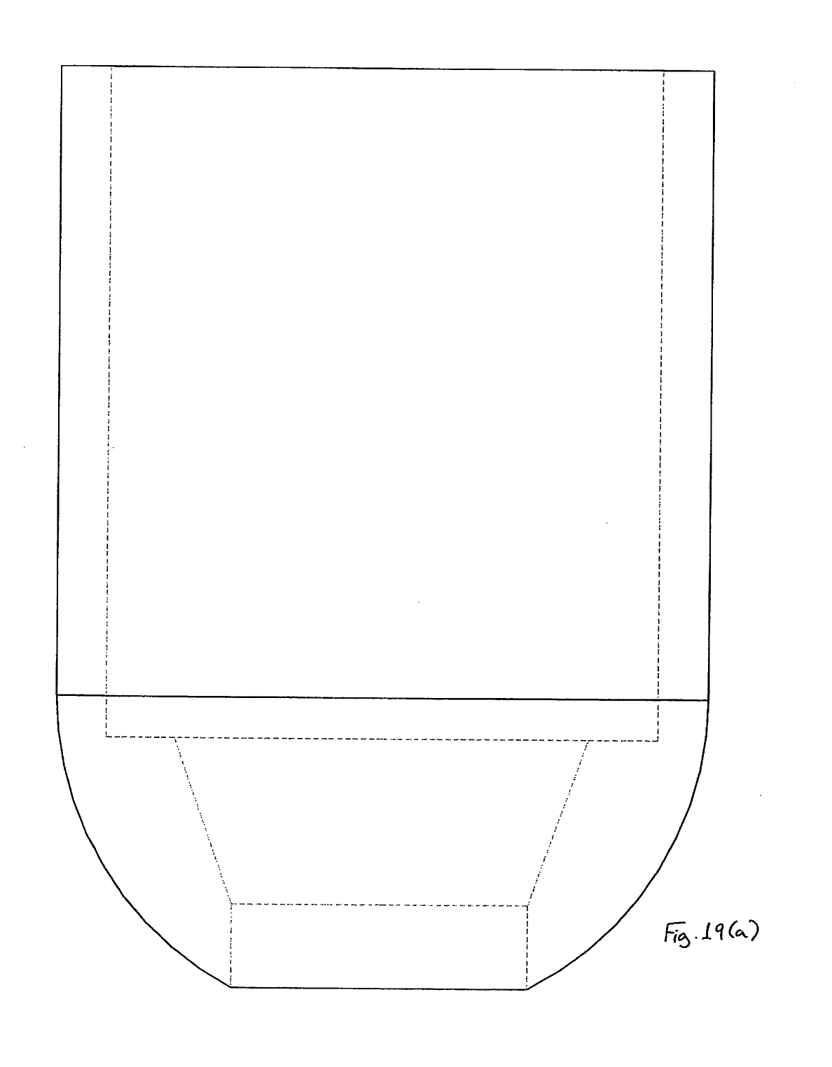

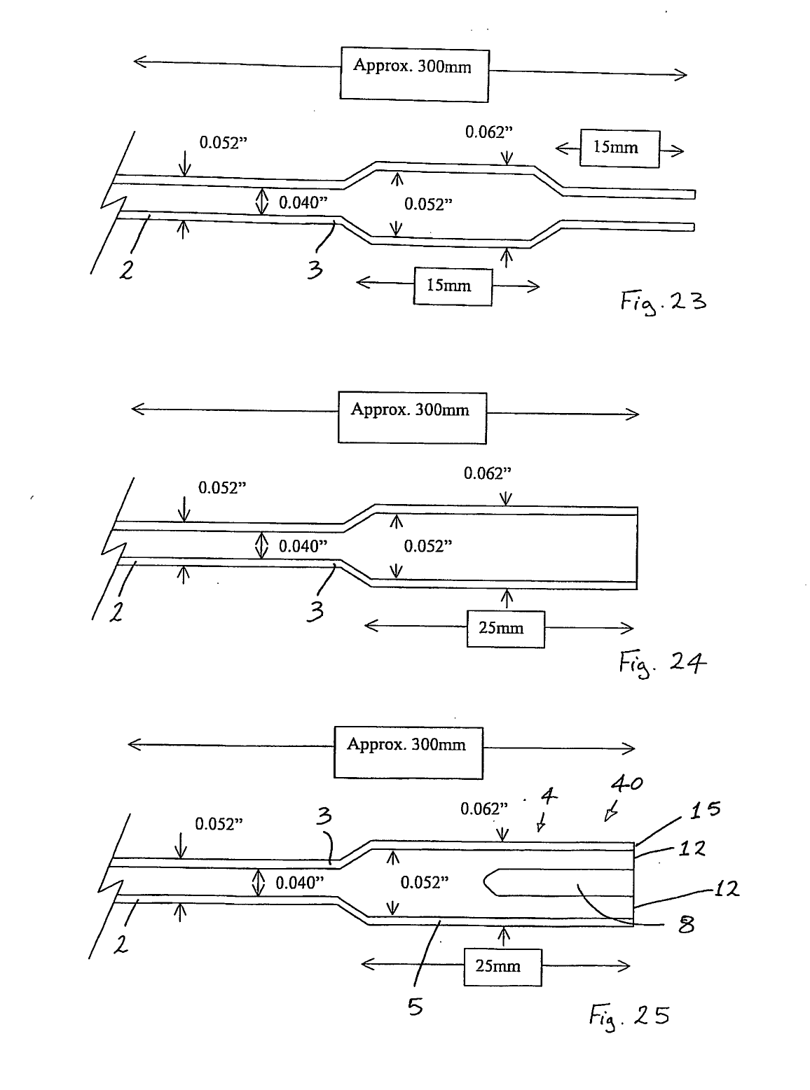

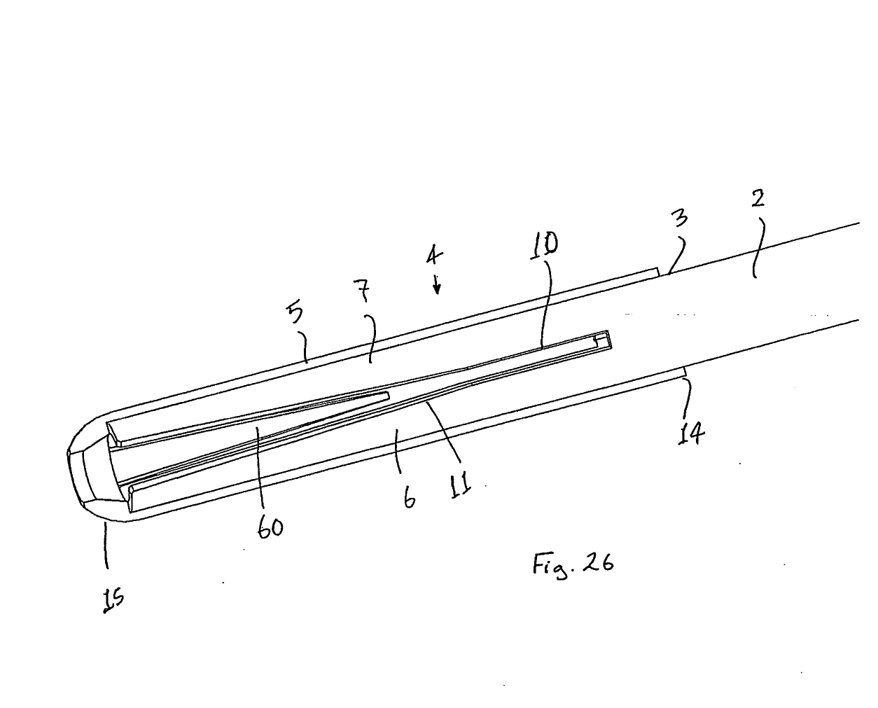

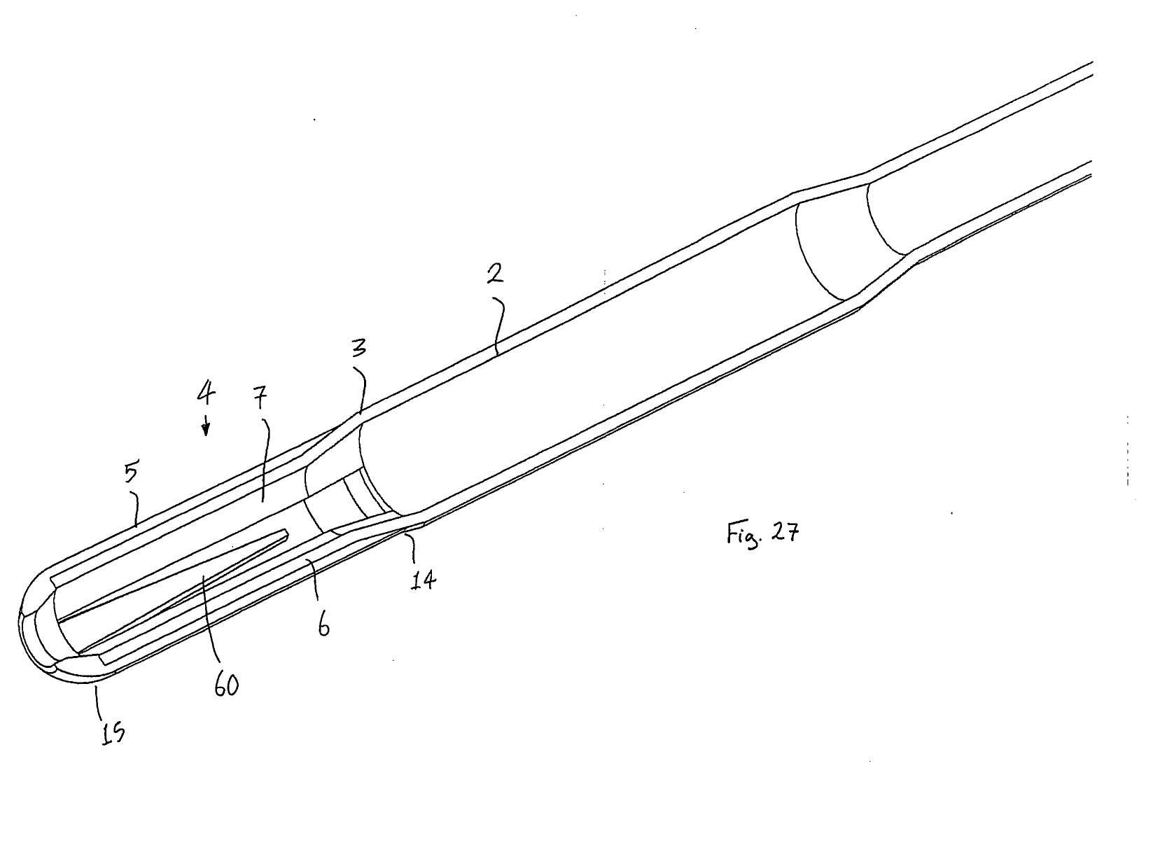

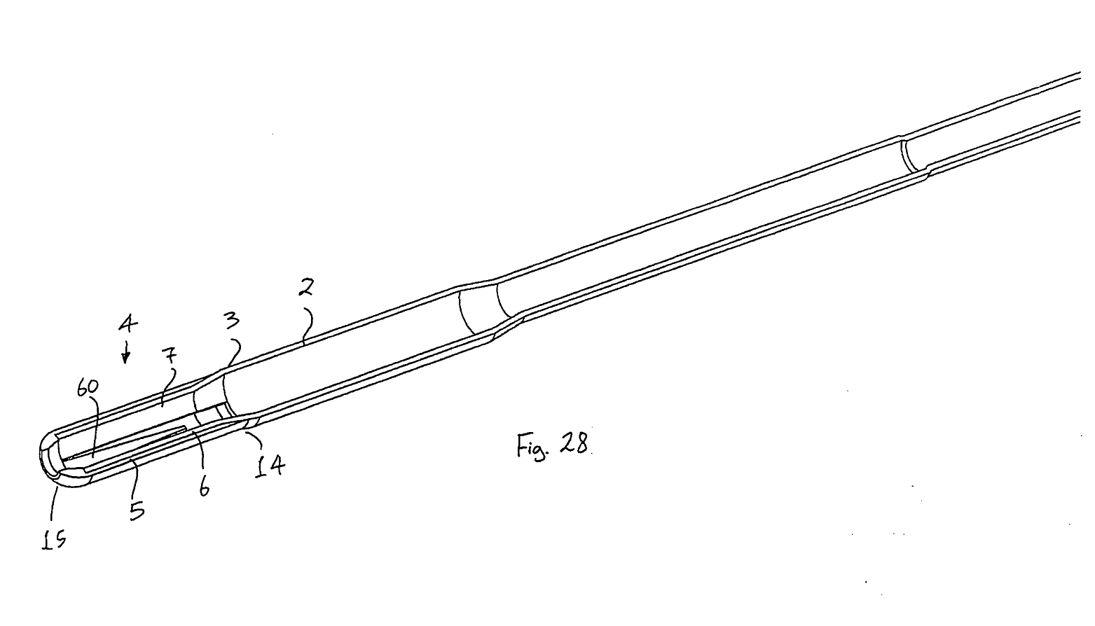

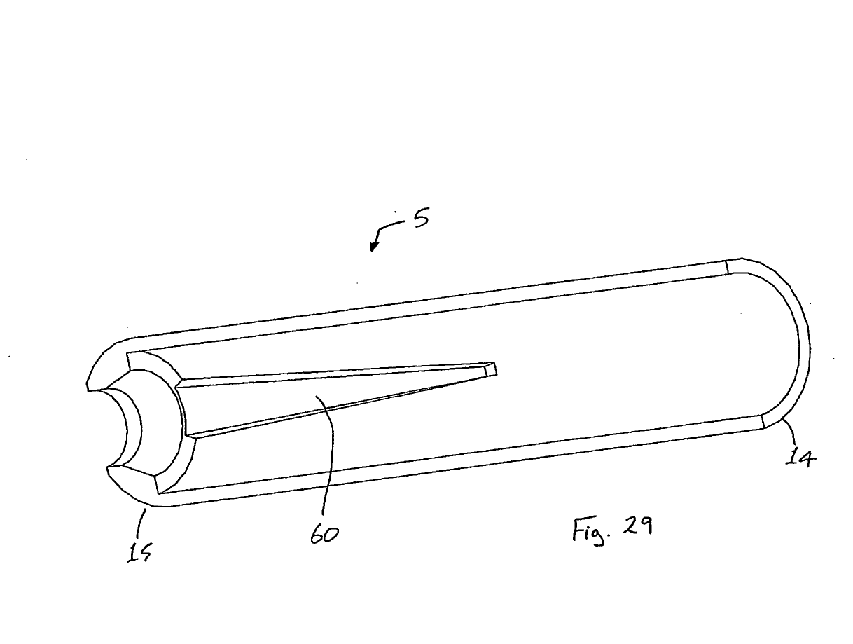

FIGS. 23 to 25 illustrate an alternative method of manufacturing a retrieval catheter 40 according to the invention.

The filter retrieval space is rigid, in this case, to constrain the outer diameter of a retrieved article, such as a retrieved embolic protection filter.

Softer material may be provided between the columns 6, 7 to maximise the internal diameter of the reception space.

In the retrieval catheter of FIGS. 26 to 29, the tip body 5 comprises two longitudinally extending ridges 60. The ridges 60 are provided on the inner surface of the tip body 5 projecting radially inwardly. The ridges 60 are aligned with the slots 8, 9 at the distal part 11 of the columns 6, 7. In this manner the ridges 60 provide a means of separating the laterally adjacent columns 6, 7. This separation may be particularly useful to prevent the columns 6, 7 from fusing together or otherwise becoming attached together during manufacture of the retrieval catheter.

The ridges 60 taper distally from a smaller circumferential dimension to a larger circumferential dimension.

As illustrated in the retrieval catheter of FIGS. 30 to 33, the columns 6, 7 may be located radially inwardly of the tip body 5, fixedly attached to the inner surface of the tip body 5.

In this case the outer diameter of the catheter shaft 2 is substantially equal to the outer diameter of the tip body 5. The columns 6, 7 taper radially inwardly at the distal end 3 of the catheter shaft 2 to pass beneath the wall of the tip body 5. In this case the columns taper from an outer diameter of approximately 0.062″ to 0.052″.

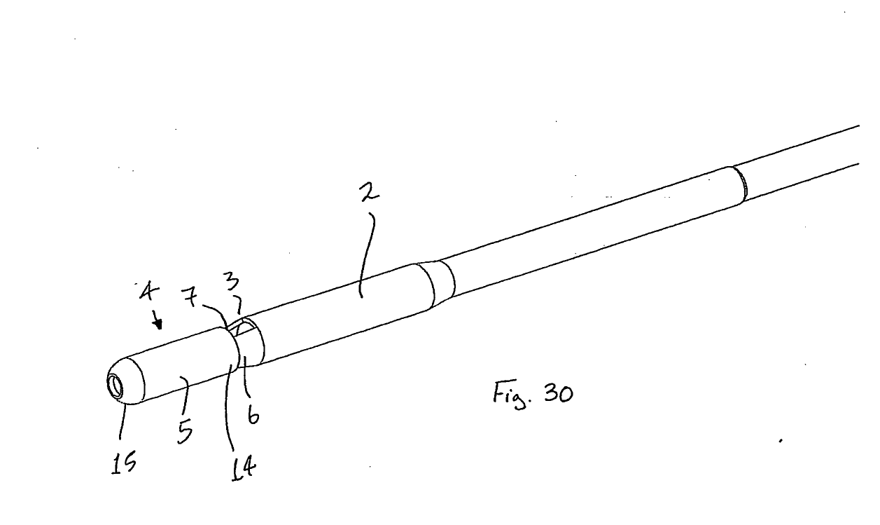

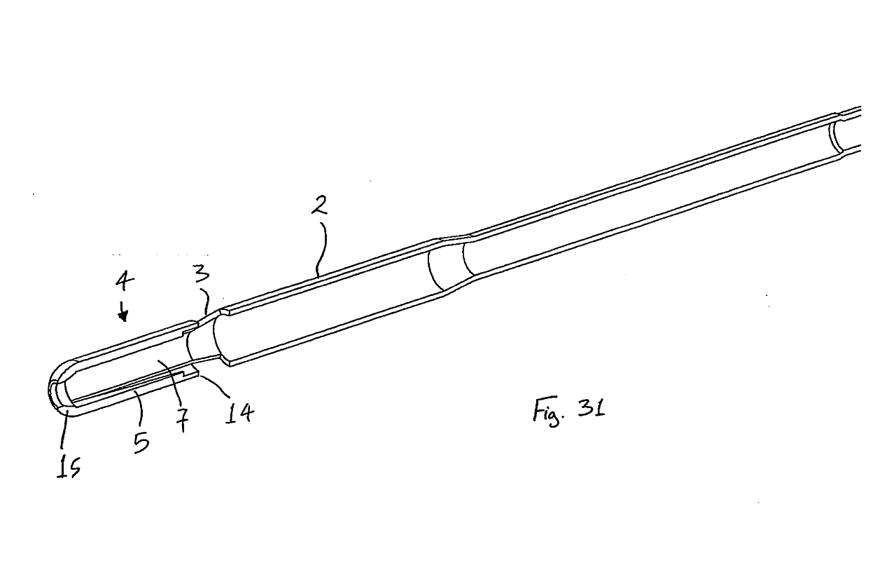

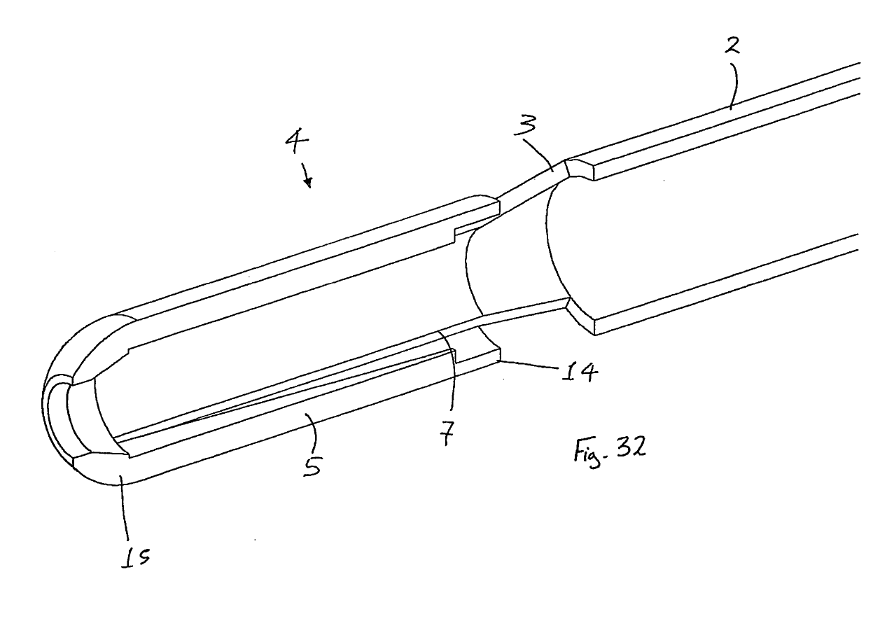

As illustrated in FIG. 32, the proximal end 14 of the tip body 5 is spaced distally of the distal end 3 of the catheter shaft 2, with the columns 6, 7 bridging this gap.

The tip 4 acts as a lead-in for the object being retrieved.

The relatively large diameter catheter shaft 2 facilitates retrieval of the object into the catheter shaft 2 in certain circumstances. In this case the catheter shaft 2 is formed of a high strength material, and has a relatively thin outer wall.

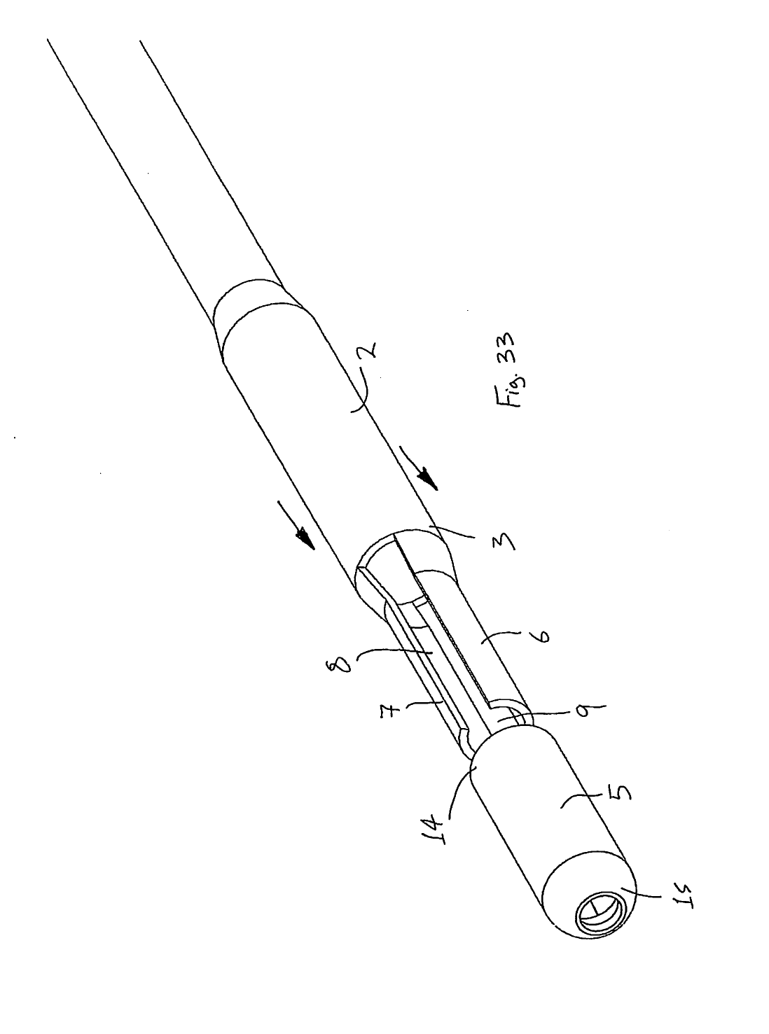

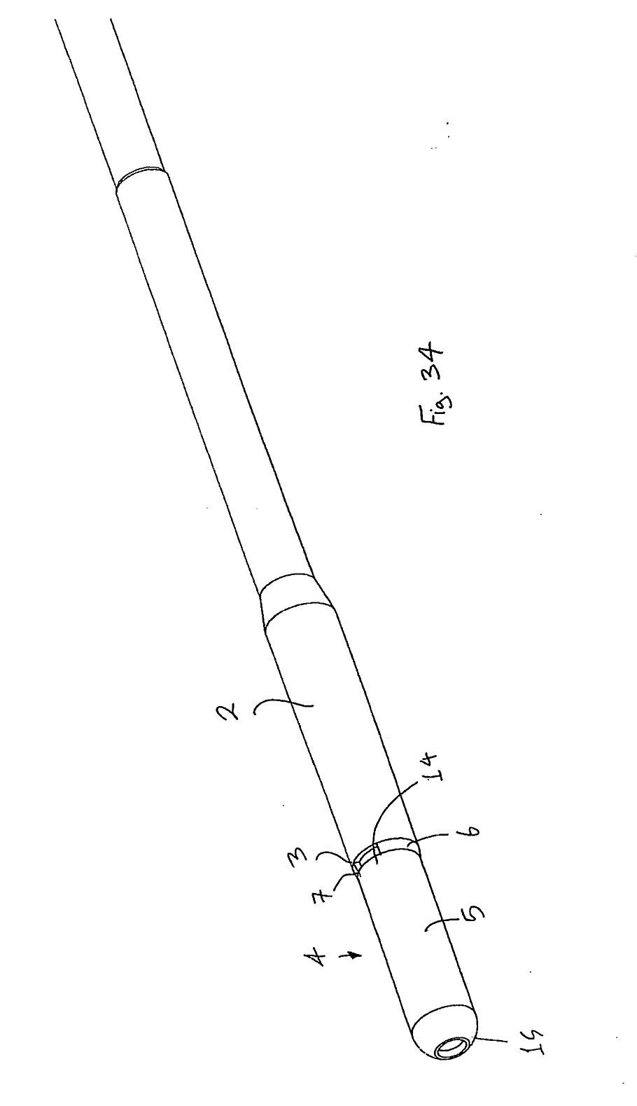

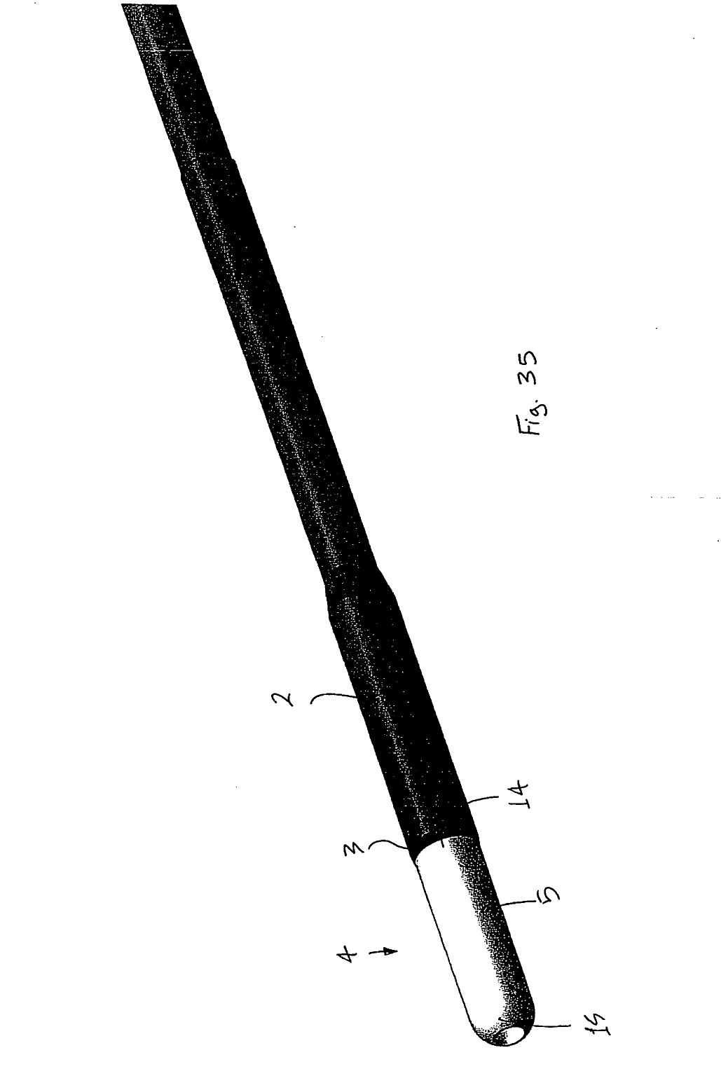

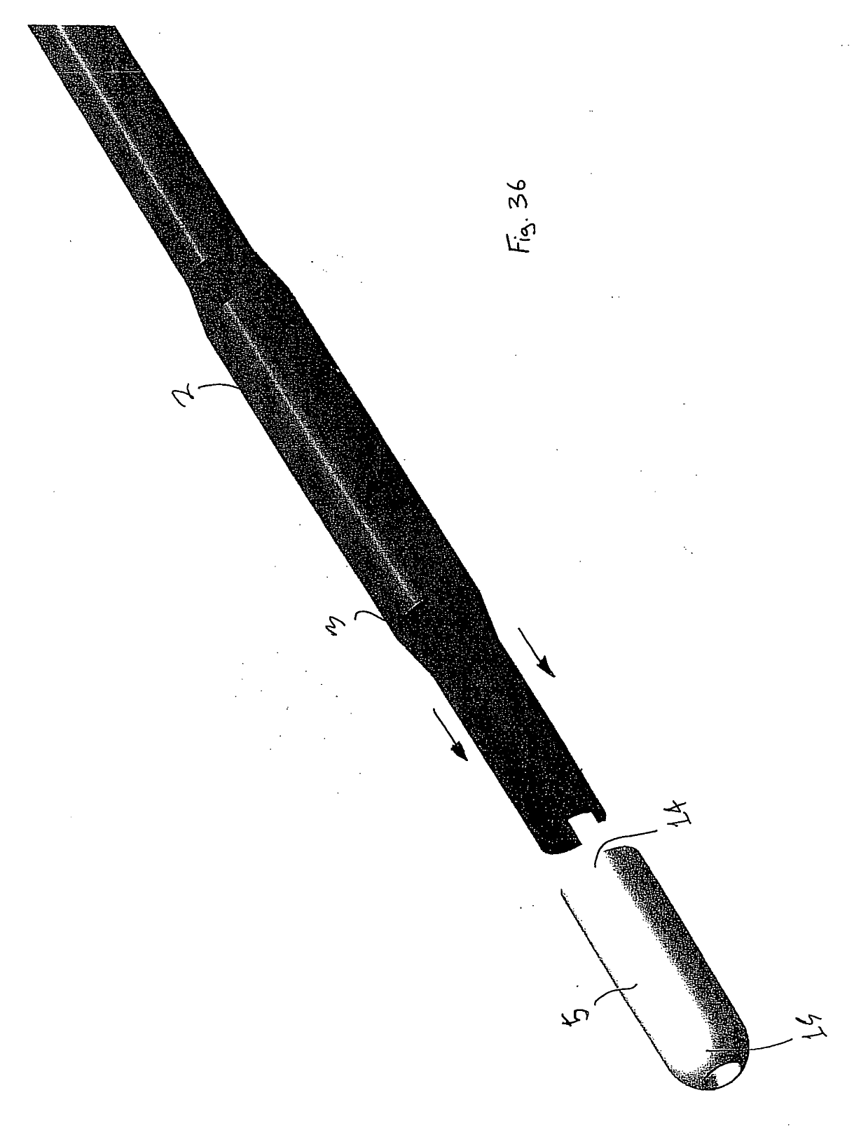

The distance between the distal end 3 of the catheter shaft 2 and the proximal end 4 of the tip body 5 may be varied to suit the requirements of the retrieval catheter. In the retrieval catheter of FIGS. 34 to 36, there is substantially no gap between the distal end 3 of the catheter shaft 2 and the proximal end 14 of the tip body 5.

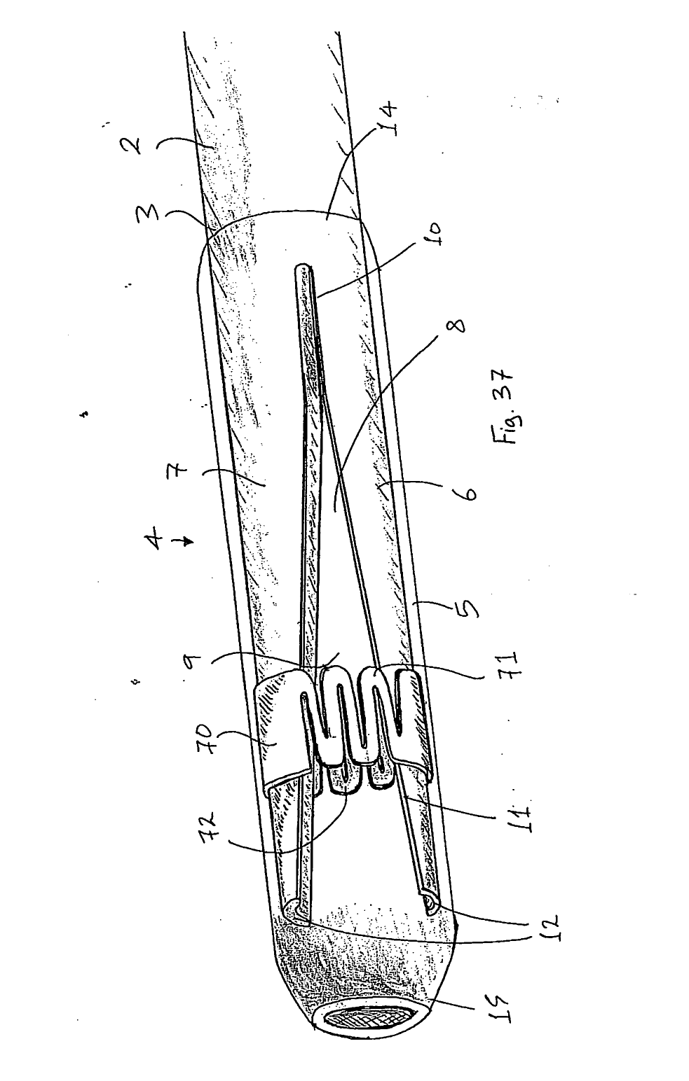

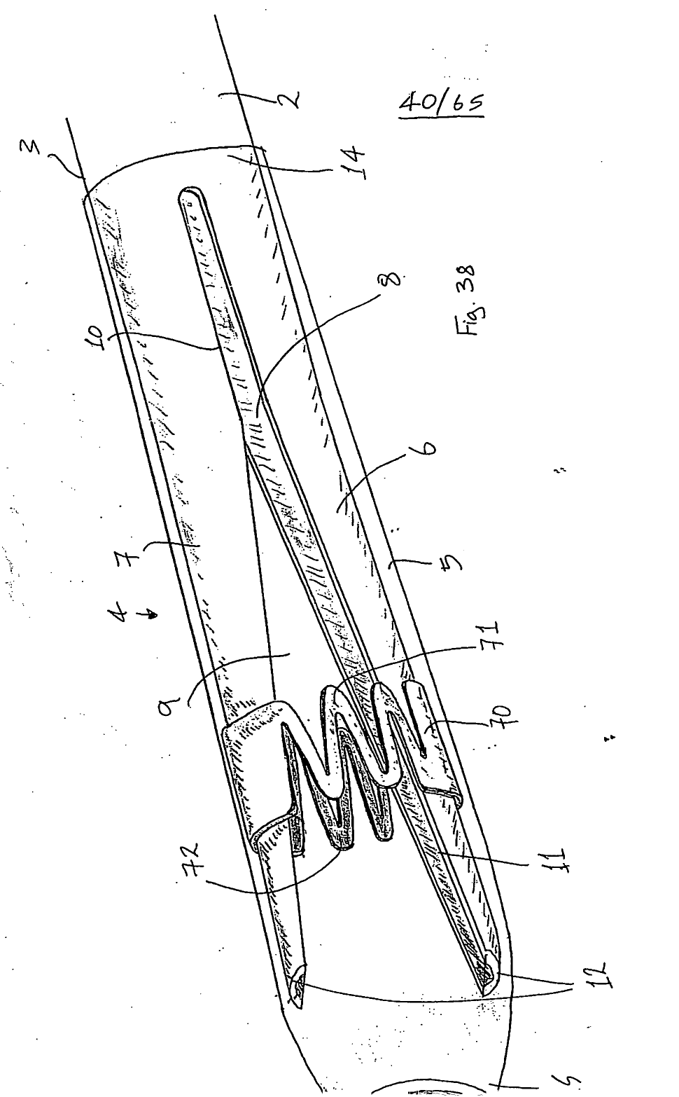

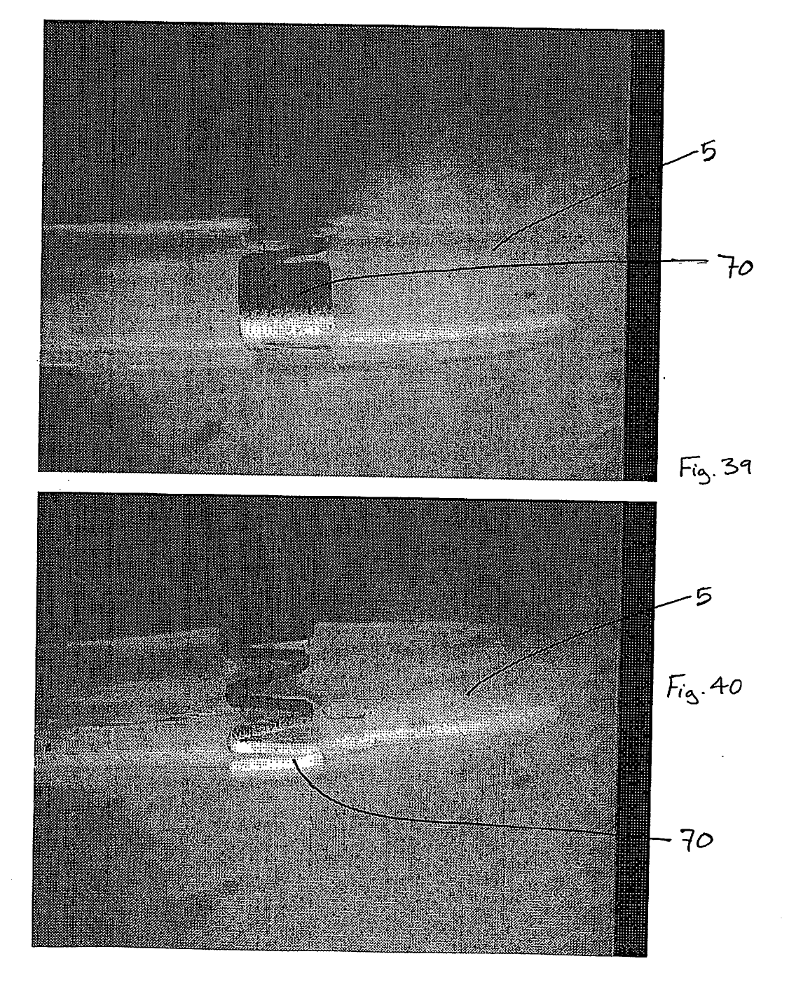

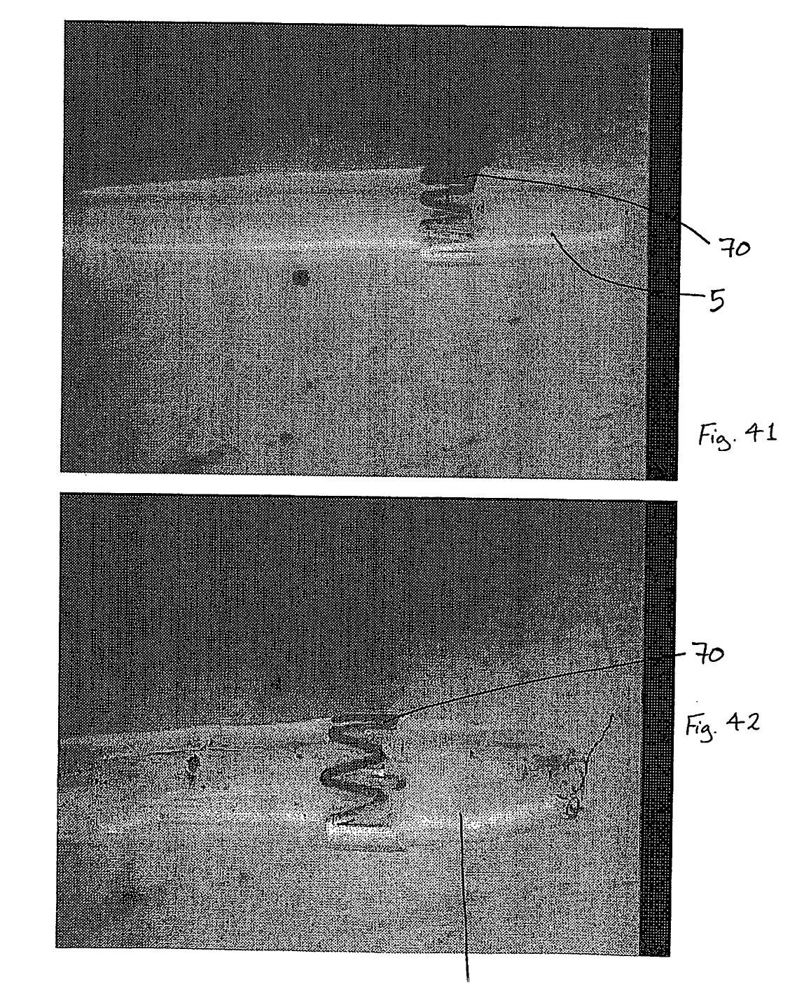

Referring to FIGS. 37 to 42, the retrieval catheter comprises a radiopaque band 70 arranged around the distal part 11 of the reinforcement columns 6, 7. The band 70 has two expandable link arms 71, 72 to facilitate radial expansion of the band 70 as the columns 6, 7 move radially upon retrieval of an object into the tip 4. The band 70 provides the tip 4 with radiopacity without adversely affecting the expansile capabilities of the tip 4.

The band 70 may be attached to the outer surface of the tip body 5, and/or to the inner surface of the tip body 5, and/or may be embedded within the tip body 5.

The band 70 may be of any suitable radiopaque material, such as tungsten or platinum.

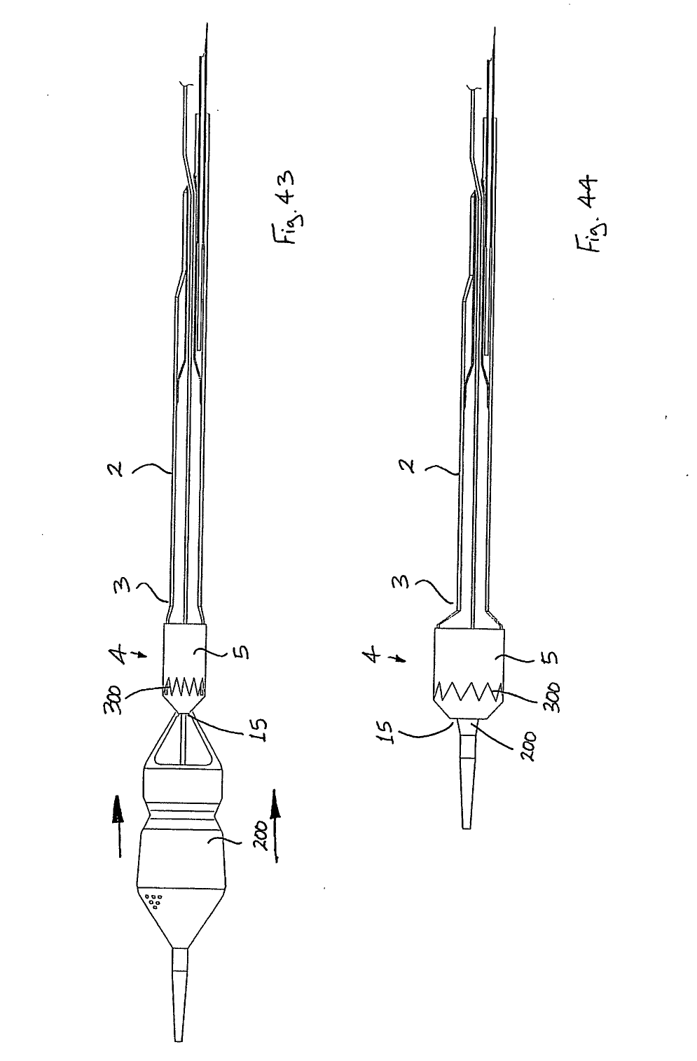

In the case of the retrieval catheter of FIGS. 43 and 44, the radiopaque band is provided in the form of a ring 300 having a zig-zag, concertina configuration. The band 300 is movable from a radially collapsed configuration (FIG. 43) to a radially expanded configuration (FIG. 44) upon retrieval of the filter 200 into the tip 4. The band 300 provides the tip 4 with radiopacity without adversely affecting the expansile capabilities of the tip 4.

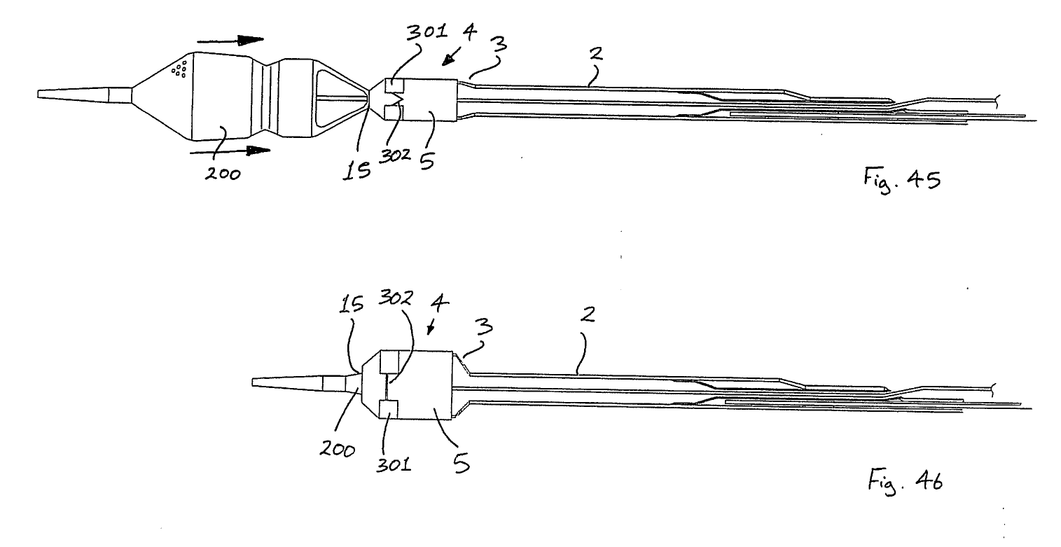

In the retrieval catheter of FIGS. 45 and 46, the radiopaque band 301 has two expandable link arms 302. The link arms 302 are movable from a collapsed configuration (FIG. 45) to an extended configuration (FIG. 46) upon retrieval of the filter 200 into the tip 4.

In the extended configuration of FIG. 46, the link arms 302 are fully extended, aligned in the radial direction, and are under tension. The link arms 302 thus act to limit any further expansion of the tip body 5. In this manner the link arms 302 act as a safety mechanism to prevent excessive expansion of the tip body 5 which could potentially lead to tearing or rupture of the tip body 5.

The retrieval catheter of FIGS. 47 and 48 is similar to the retrieval catheter of FIGS. 45 and 46. In this case, the radiopaque band 303 comprises a mechanical lock arrangement to limit any further expansion of the tip body 5, when the band 303 is in the expanded configuration of FIG. 48.

It will be appreciated that the tip body 5 may be attached to the reinforcement elements 6, 7 in a variety of different configurations. For example in the retrieval catheter of FIGS. 1 to 11, the reinforcement elements 6, 7 are embedded within the tip body 5, and the tip body 5 is fixedly attached to the reinforcement elements 6, 7 along the full longitudinal length of the elements 6, 7 and around the full circumferential dimension of the elements 6, 7.

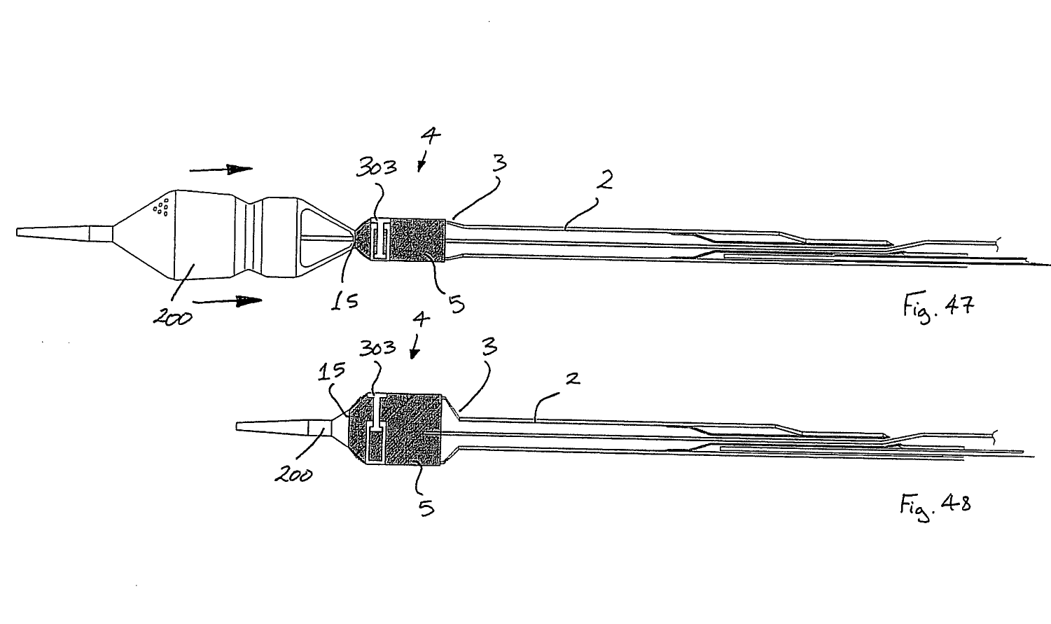

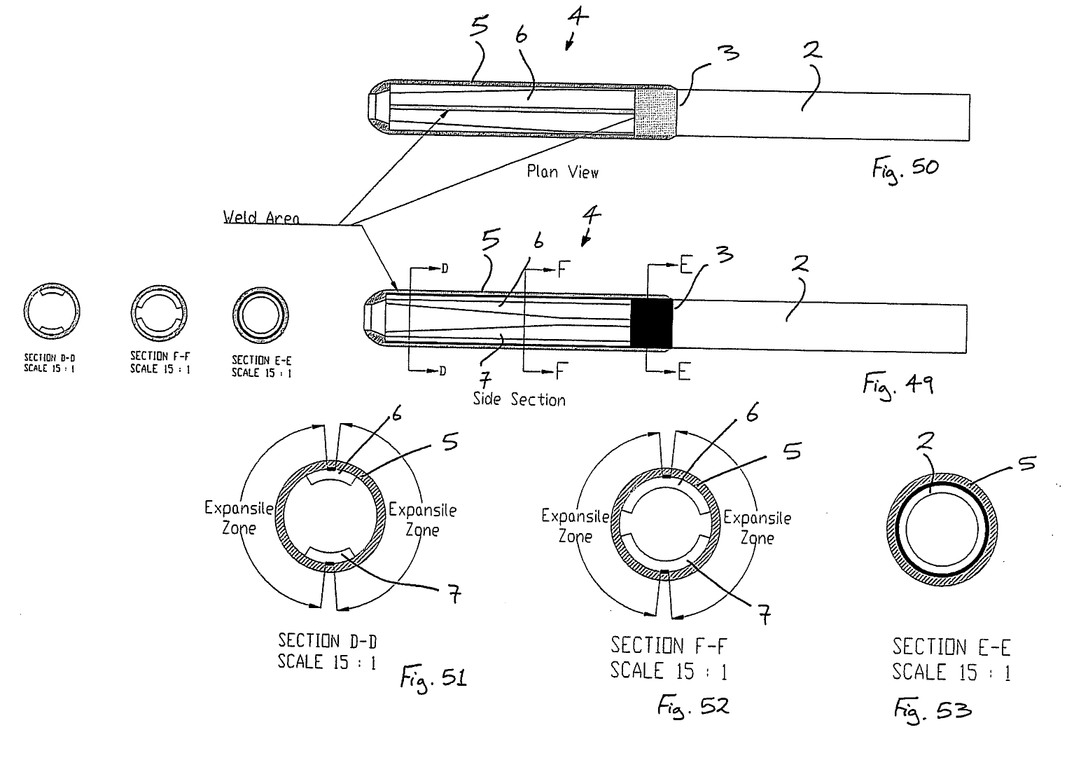

An alternative attachment arrangement is illustrated in FIGS. 49 to 53. In this case the tip body 5 is fixedly attached to the reinforcements elements 6, 7 along the full longitudinal length of the elements 6, 7. However, as illustrated in FIGS. 51 and 52, the tip body 5 is fixedly attached to the reinforcement elements 6, 7 around only a portion of the circumferential dimension of the elements 6, 7. At the proximal end of the tip 4, the tip body 5 is fixedly attached to the distal end 3 of the catheter shaft 2 around the full circumferential dimension of the shaft 2 (FIG. 49).

By limiting the circumferential extent by which the tip body 5 is attached to the reinforcement elements 6, 7, this arrangement increases the expansile zone of the tip body 5, and this increases the overall expansile capabilities of the tip 4.

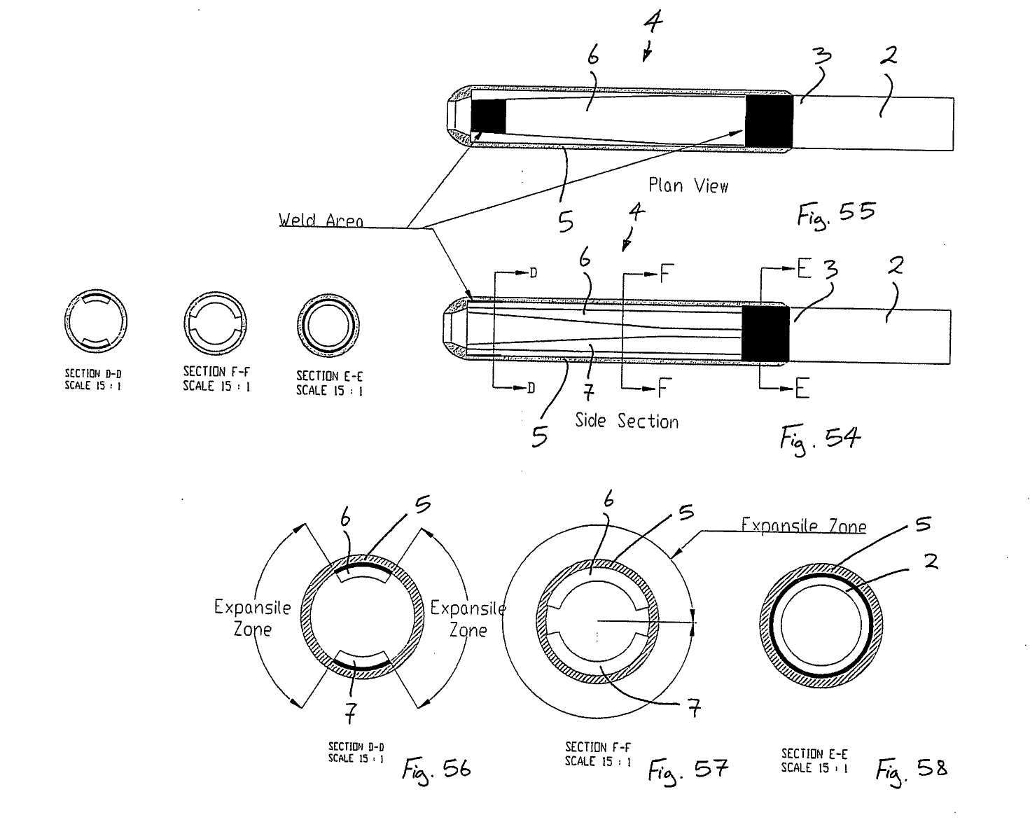

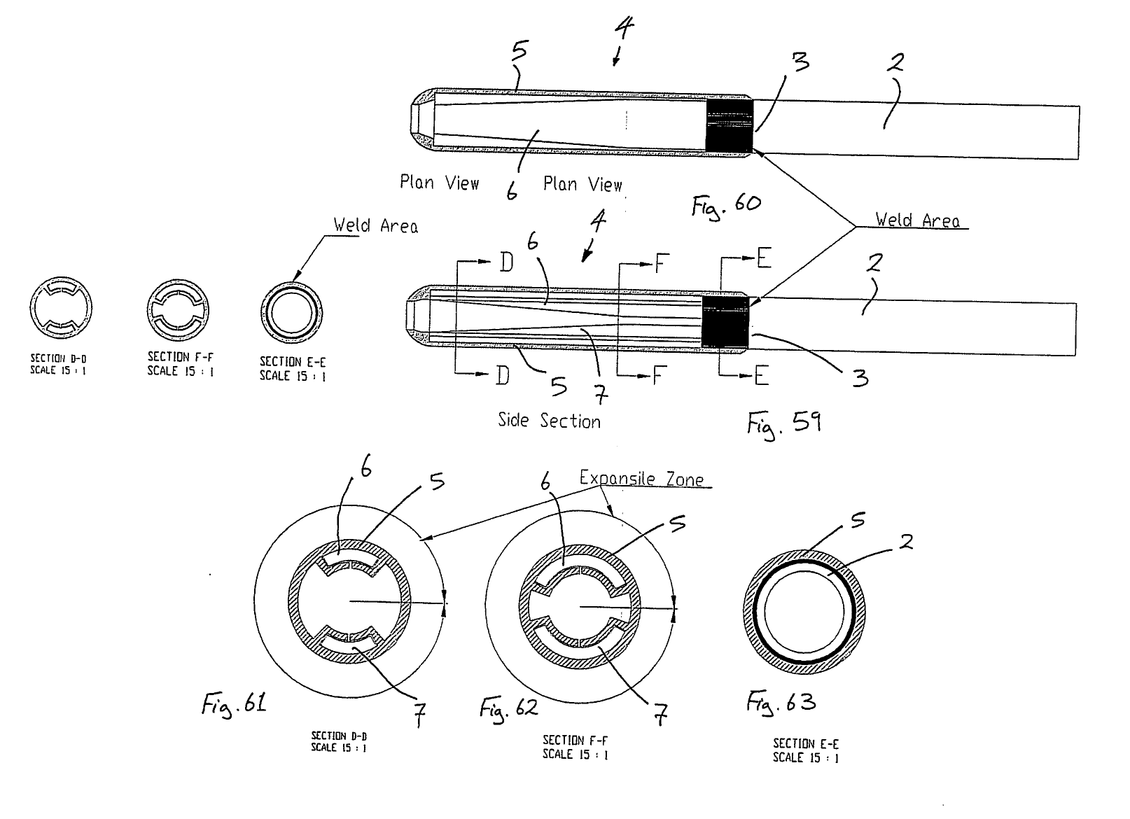

In the retrieval catheter of FIGS. 54 to 58, the tip body 5 is fixedly attached to the reinforcement elements 6, 7 at the distal end 15 of the tip 4, and the tip body 5 is fixedly attached to the distal end 3 of the catheter shaft 2. Along the remaining length of the reinforcement elements 6, 7, the tip body 5 remains detached from the reinforcement elements 6, 7. At the distal end 15 of the tip 4, the tip body 5 is fixedly attached to the reinforcement elements 6, 7 around the full circumferential dimension of the reinforcement elements 6, 7.

As illustrated in FIGS. 56 and 57, the expansile zone of the tip body 5 is increased along the detached portion of the reinforcement elements 6, 7.

It is not essential that the tip body 5 be fixedly attached to the reinforcement elements 6, 7. For example, and as illustrated in FIGS. 59 to 63, the tip body 5 may be coupled to the reinforcement elements 6, 7 without directly attaching the tip body 5 to the reinforcement elements 6, 7. In this case, the tip body 5 extends over the outer surface of the reinforcement elements 6, 7 and over the inner surface of the reinforcement elements 6, 7, so that the reinforcement elements 6, 7 are embedded within the tip body 5 (FIGS. 61 and 62). At the distal end 3 of the catheter shaft 2, the tip body 5 is fixedly attached to the catheter shaft 2.

By arranging for the tip body 5 to be detached from the reinforcement elements 6, 7 along the full length of the reinforcement elements 6, 7, this increases the expansile zone of the tip body 5.

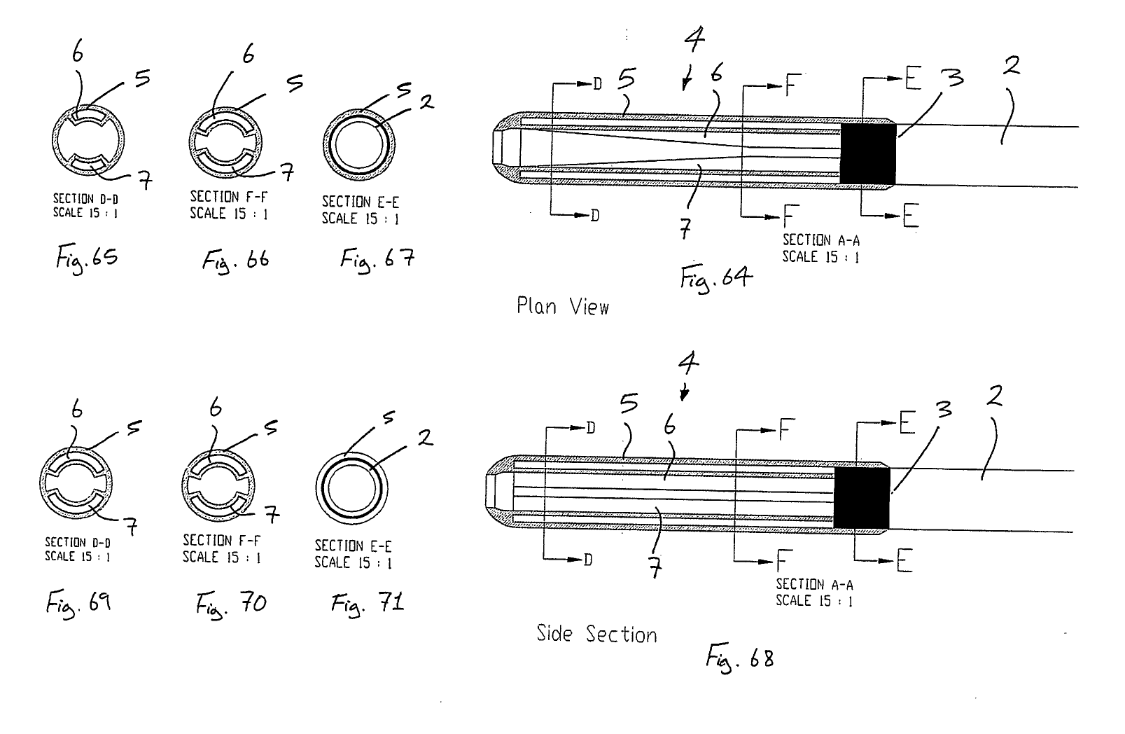

FIGS. 64 to 71 illustrate further retrieval catheters similar to the retrieval catheter of FIGS. 59 to 63. The tip body 5 may be arranged to be detached from the reinforcement elements 6, 7 in the case of reinforcement elements 6, 7 which taper distally from a larger circumferential dimension to a smaller circumferential dimension (FIGS. 64 to 67), or in the case of reinforcement elements 6, 7 whose circumferential dimension is constant along the length of the reinforcement elements 6, 7 (FIGS. 68 to 71).

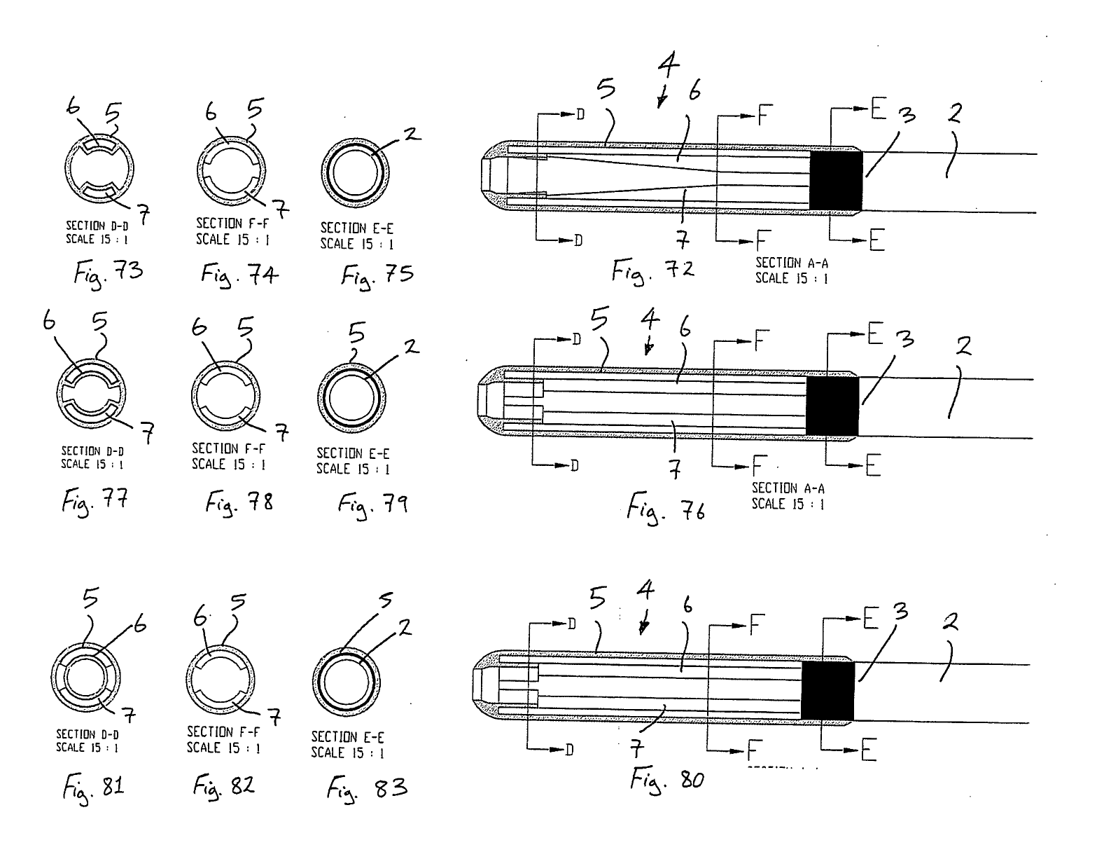

It will be appreciated that the tip body 5 may be coupled to the reinforcement elements 6, 7 along only a portion of the longitudinal length of the reinforcement elements 6, 7. For example, and as illustrated in FIGS. 72 to 83, only the distal end of the reinforcement elements 6, 7 may be embedded within the tip body 5. Along the remaining length of the reinforcement elements 6, 7, the tip body 5 extends along the exterior surface of the reinforcement elements 6, 7 but the reinforcement elements 6, 7 are not embedded within the tip body 5.

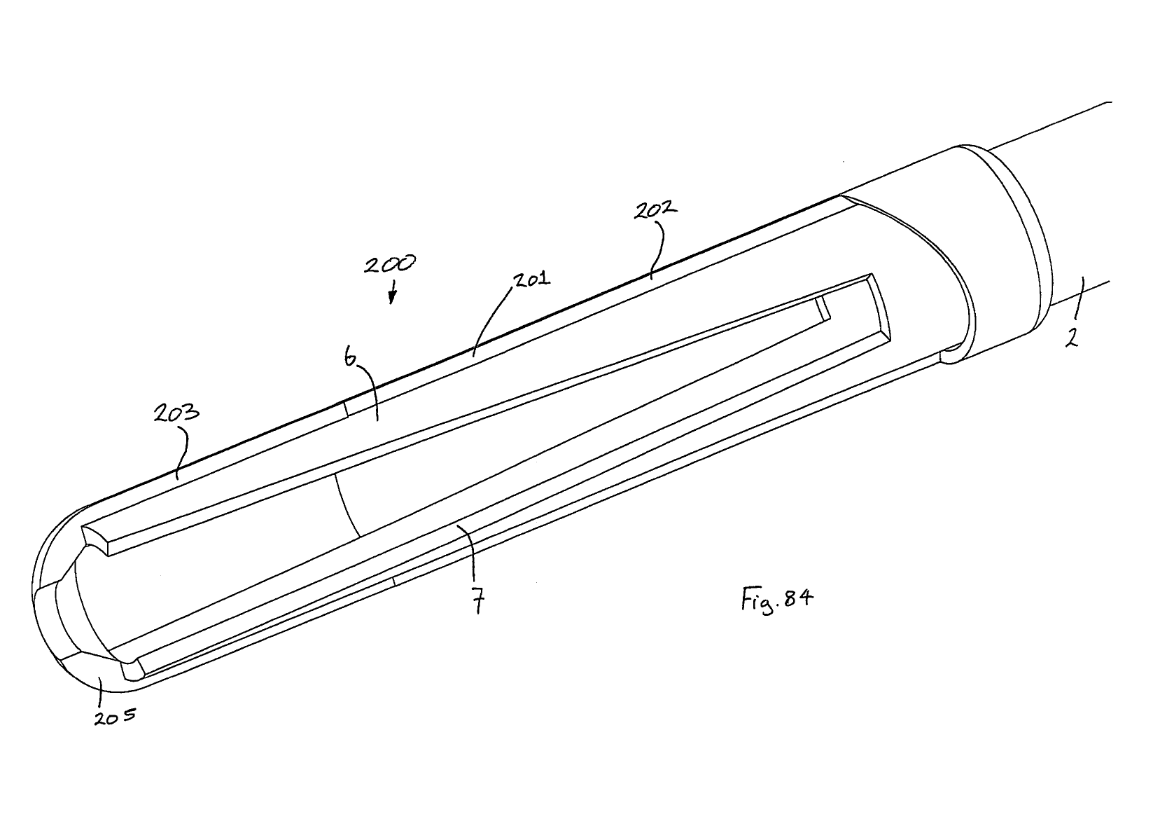

The retrieval catheter 200 of FIG. 84 has a tip body 201 with a soft proximal tubular segment 202 and a stiff, radiopaque distal tubular segment 203.

In this case the distal segment 203 comprises C-FLEX R70-089 (Hardness=shore 51A, Tear strength (ppi)−268)+70 wt % TELEDYNE C-5 extrusion grade TUNGSTEN+0.5 wt % IRGANOX 1010+3 wt % PERFLUOROPOLYETHER.

In this case the proximal segment 202 comprises C-Flex MHR 50A R70-091-000

Hardness-Shore 51 A

Tear strength (ppi)-242

This c-flex is harder than the C-flex used in the radiopaque tip 203 but is softer than the tip c-flex with radiopaque filler. Higher tear strength and % elongation at break is required here, as the material between the splines 6, 7 needs to stretch more due to the proximally decreasing spline cut space.

The material is hard enough to prevent the retrieval tip 201 expanding to an outer diameter greater than the internal diameter of a 7FR guide catheter when presented with excessive embolic loads. The reduced spline cut space also contributes a compressive load to the retrieved filter further preventing it from over expanding.

Extra material is provided at the tip nose 205 to allow it to stretch more than twice its original size.

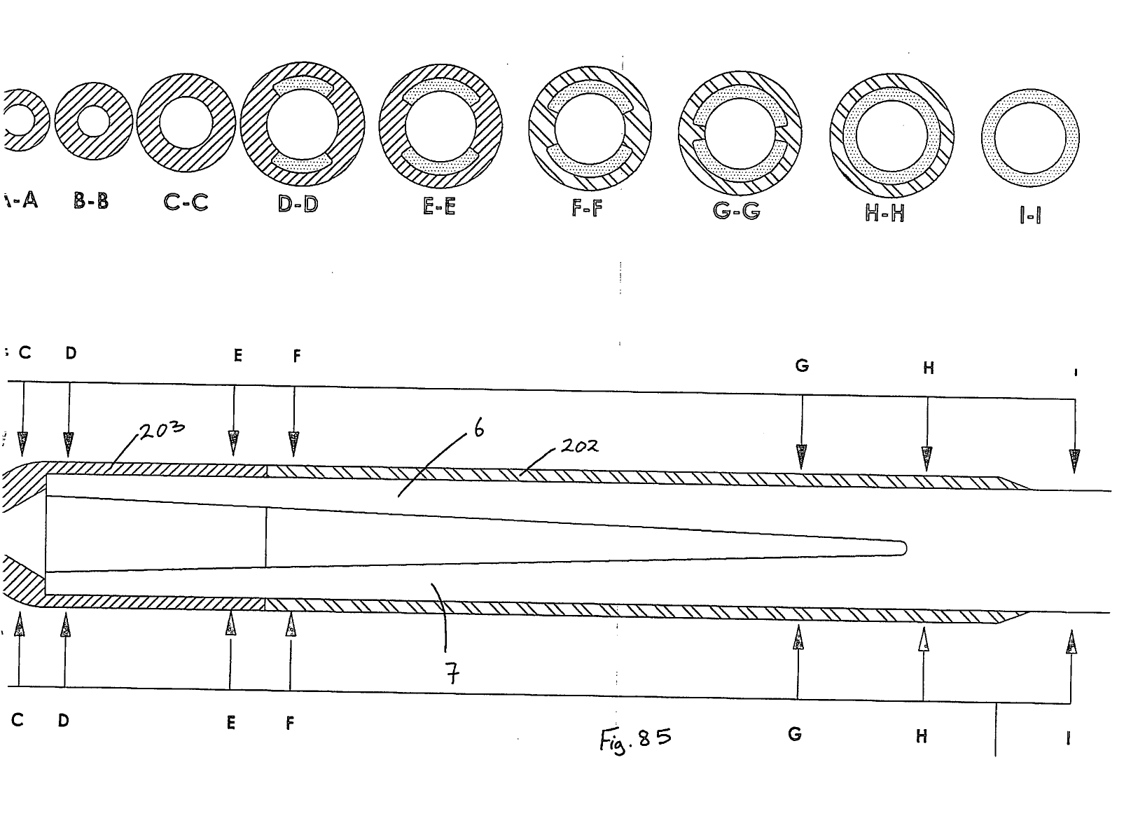

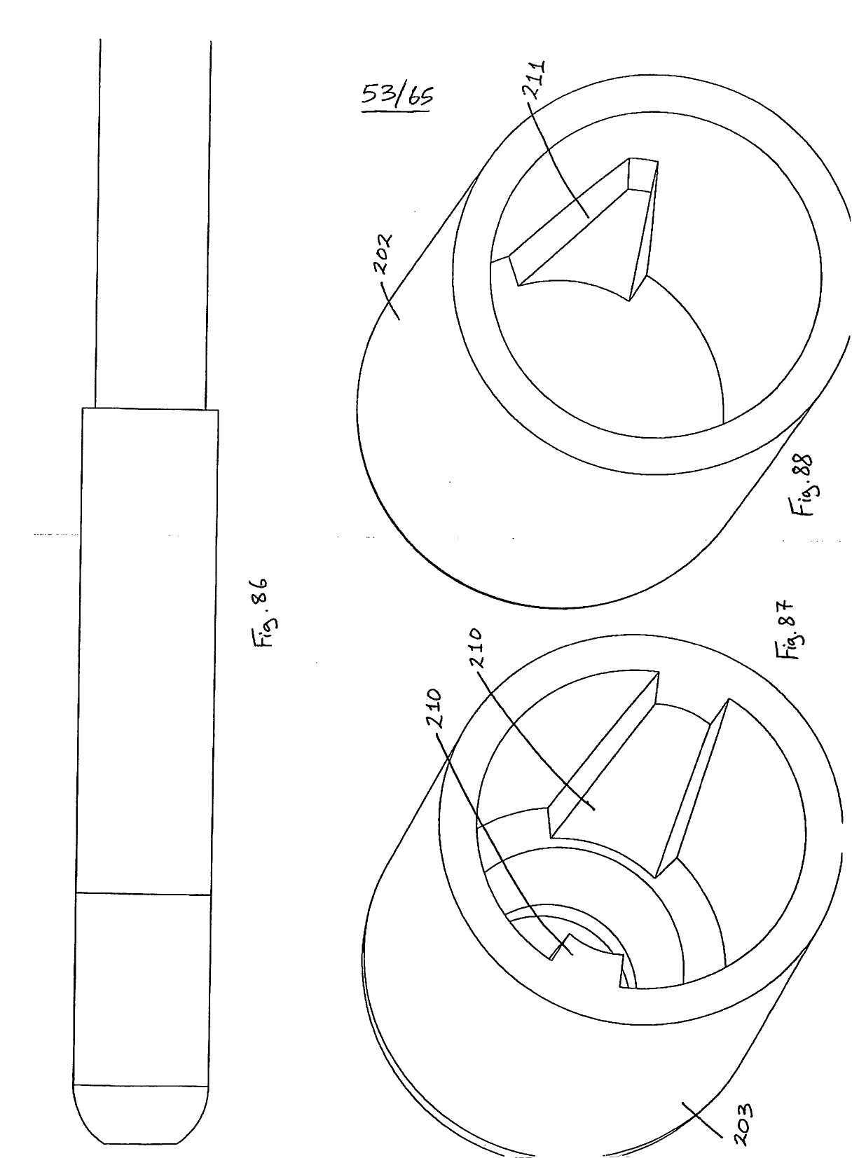

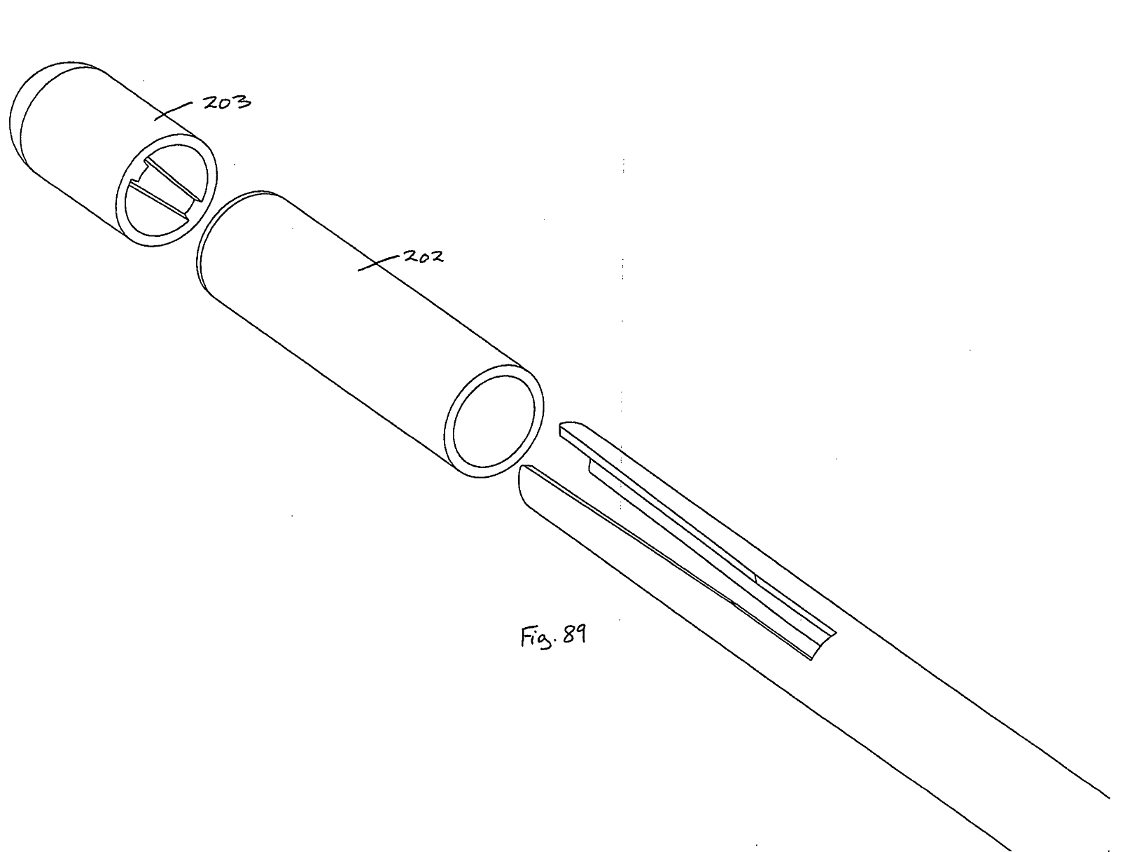

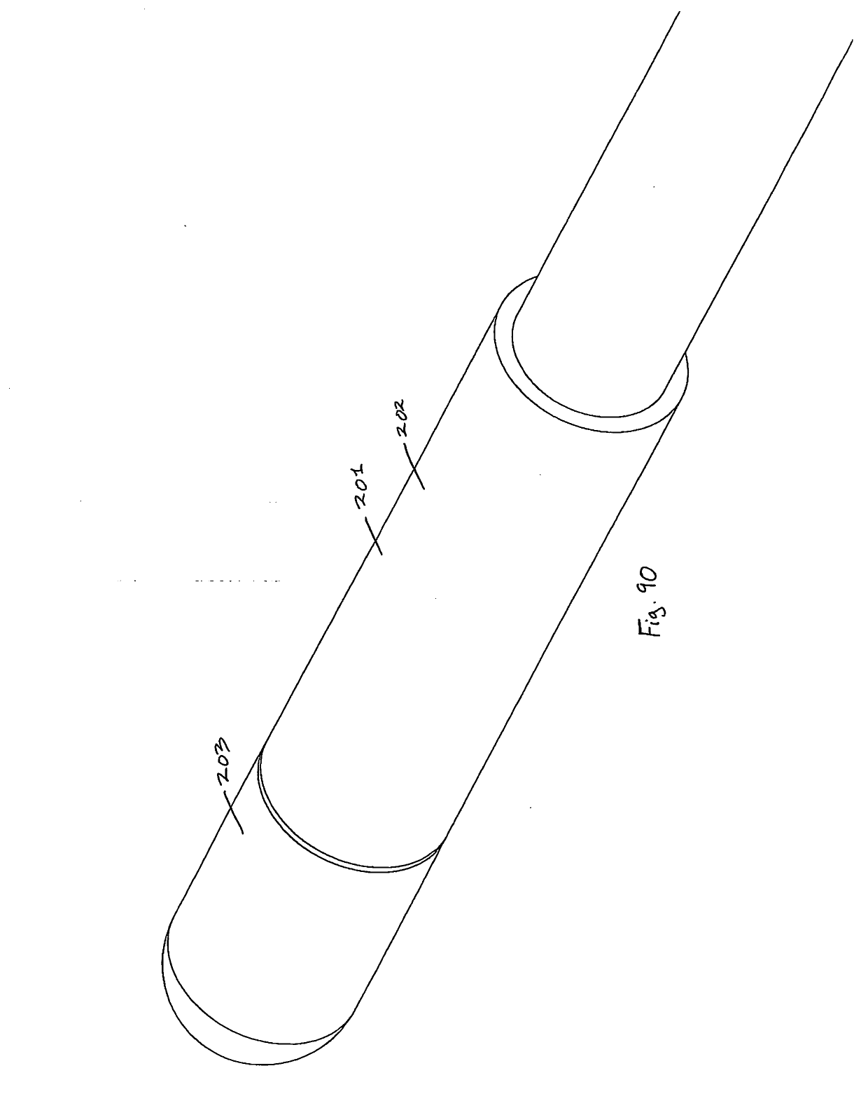

As illustrated in the cross-sections in FIG. 85, the material between the splines 6, 7 needs to stretch more as the sections move proximally.

FIG. 85 illustrates the radiopaque tip (c-flex with 70% tungsten) 203, the clear c-flex 202, and the splines 6, 7. In the distal nose 205, the area of the tip body material is greater than the area of the tip body material along the splines 6, 7. The diameter of the distal opening 250 in the distal nose 205 is approximately 0.030″. The retrieval catheter 200 is suitable for exchange over a guidewire with a diameter of approximately 0.014″.

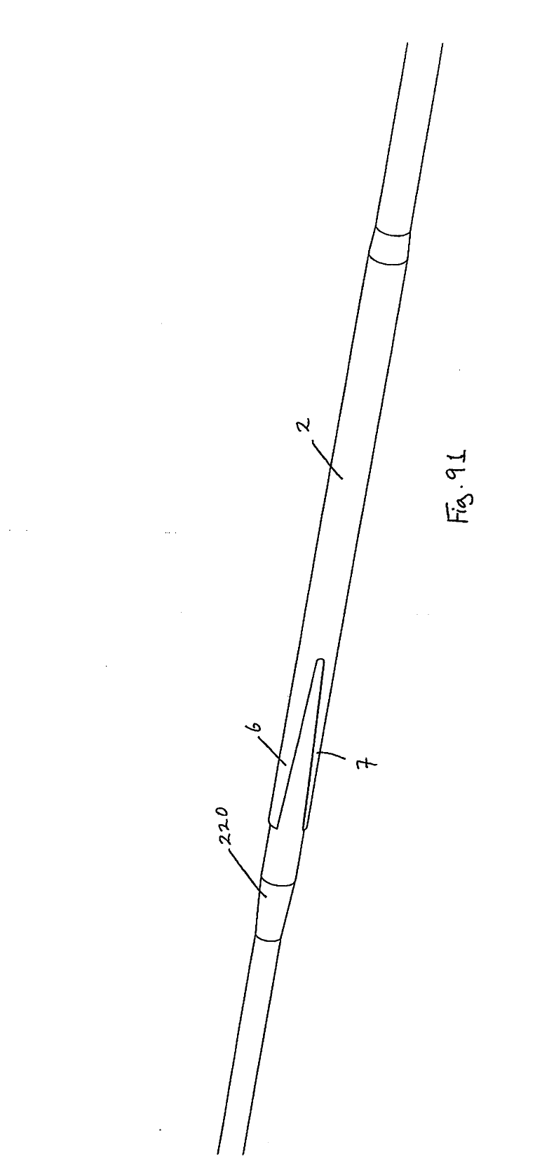

As illustrated in FIG. 87, the c-flex with radiopaque filler 203 has extra material 210 to fit between the splines 6, 7. Similarly the clear c-flex 202 has extra material 211 to fit between the splines 6, 7, as illustrated in FIG. 88.

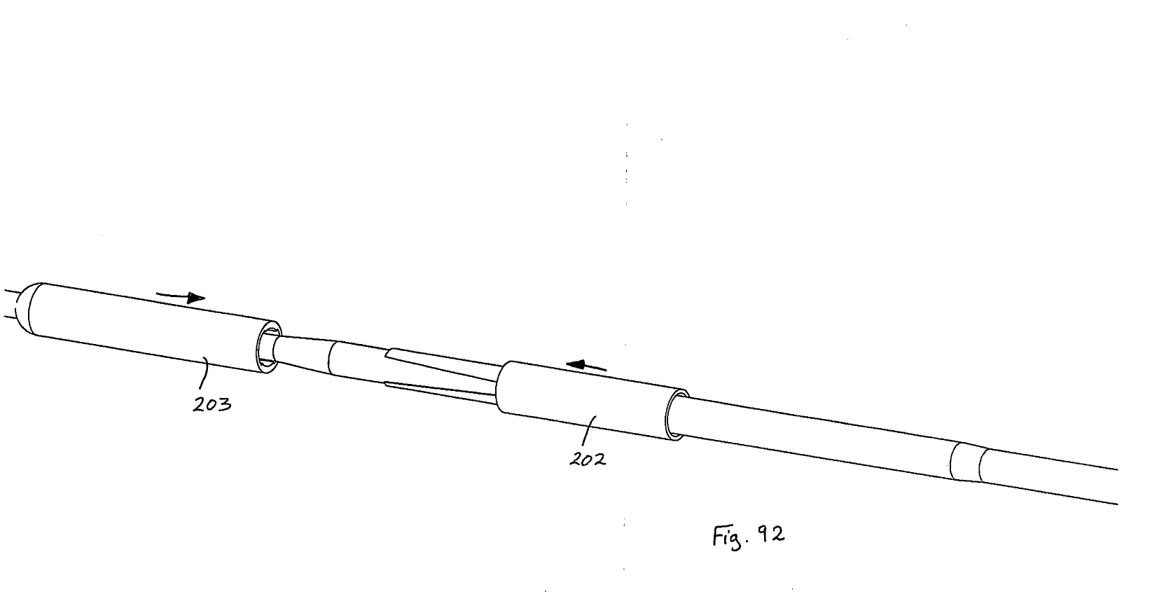

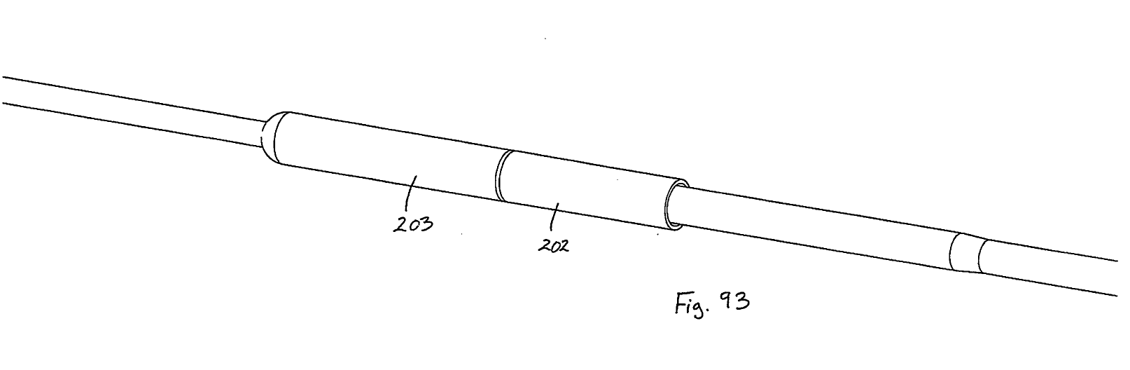

FIG. 89 illustrates the dual tip parts 202, 203 before assembly. FIG. 90 illustrates the dual tip 201 assembled.

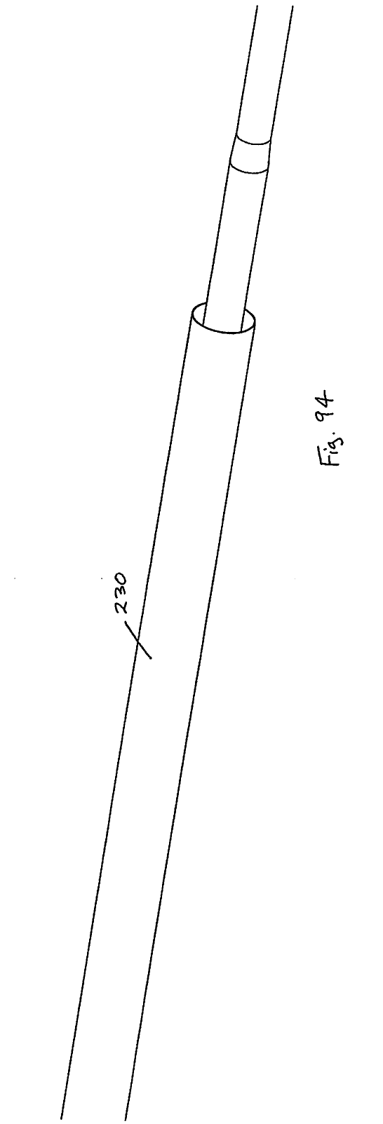

FIGS. 91 to 94 illustrate manufacture of the retrieval catheter 200. The catheter shaft 2 and the splines 6, 7 are arranged around an alignment mandrel 220 (FIG. 91). The distal radiopaque segment 203 is advanced over the mandrel 220 in the proximal direction and the proximal segment 202 is advanced over the mandrel 220 in the distal direction (FIG. 92). The distal segment 203 is bonded to the proximal segment 202 (FIG. 93).

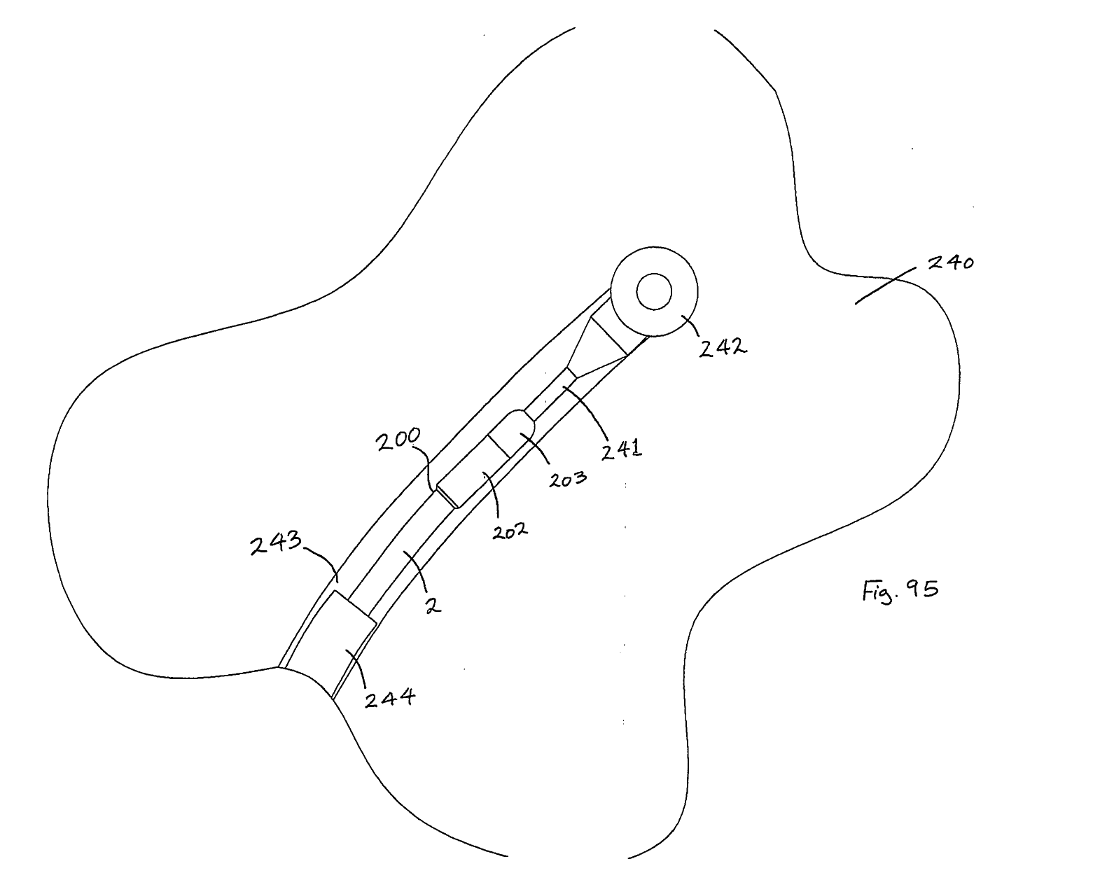

A heat shrink sheath 230 is arranged around the segments 202, 203 to hold the segments 202, 203 in place during moulding (FIG. 94). The assembly is heated until the splines 6, 7 and the two segments 201, 203 are moulded together.

To flush the retrieval catheter 200, a flushing arrangement mounted to a tray 240 may be employed, as illustrated in FIG. 95.

In particular FIG. 95 illustrates the retrieval/delivery catheter 200, a flushing tip 241, a flat surface of the tray 240, a flushing tip luer 242, a channel 243 formed into the flat tray surface, and a coil 244. The coil 244 and flush tip 241 clip into the channel 243.

The ability to differentiate materials under x-ray fluoroscopy depends on the degree to which these materials attenuate the x-rays passing through them. Attenuation is the removal of photons from a x-rays beam as it passes through matter, the greater the difference in x-ray attenuation between two materials the easier it becomes to differentiate between them in an X-ray image.

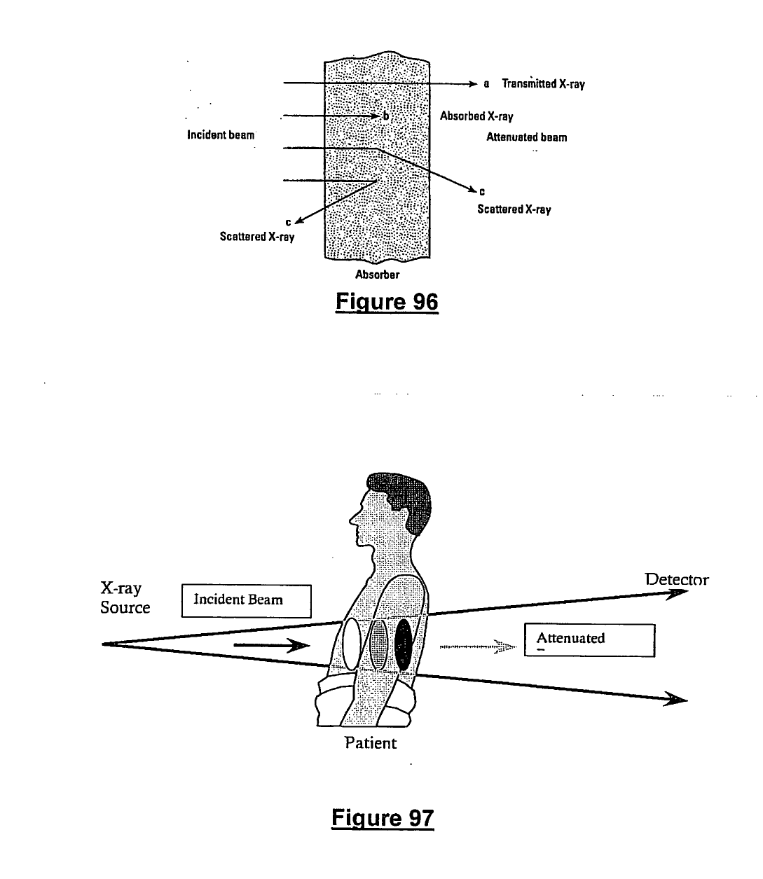

FIG. 96, demonstrates the possible outcomes for an x-ray incident on a single section of material. Attenuation of the x-ray photons is a result of absorption or scatter of the incident photons.

X-ray attenuation processes are determined by the laws of probability, the relative x-ray interaction probabilities make it possible to predict the fraction of x-rays that will penetrate a material.

In interventional x-ray imaging the x-ray beam passes through a number of materials prior to reaching the detector. As represented in FIG. 97, the materials include component materials of the medical device, muscle, bone, lung and other tissues. The radiopacity of a medical device is directly related to its level of x-ray attenuation relative to surrounding materials. One of the primary aims of interventional medical device design is that a clinical user can easily identify relevant device features under x-ray fluoroscopy.

The overall attenuation of an x-ray path composed of a number of materials has been shown to be equal to the sum of individual material attenuations along that path. For a medical device to be highly radiopaque its relative x-ray attenuation level, compared to the other materials surrounding it, must be high.

Historically, interventional catheter radiopacity was achieved by incorporating the following into the catheter construction:
(a) Ridged metallic components with high x-ray attenuation coefficients or,
(b) Thicker walled components with moderate attenuation coefficients, i.e. greater than typical physiological materials but lower than metallic elements/alloys.

Some fundamental problems exist with these approaches to making a retrieval catheter tip designs radiopaque:
1. Neither is compatible with an easily deformable/expansile tip—limiting the optimisation of retrieval and trackability performance.
2. Neither is compatible with achieving a smooth, profiled, radiopaque end transition of the catheter tip, which is desirable in terms of catheter delivery—particularly stent crossing.
3. Neither is compatible with achieving adequate x-ray attenuation to the very distal end of an expandable retrieval catheter tip.

Embolic protection devices are currently employed during Carotid Stenting and during the stenting of SVG stenting. Clinical experience to date has shown that a lot of the device complications associated with these procedures occur during the retrieval step. The ability to clearly identify the distal end of a catheter will help reduce these complications. These complications occur for a number of reasons:
Retrieval catheters must cross over freshly implanted stents to retrieve the filter. Freshly implanted stents are more hazardous to cross as they have not had an opportunity to embed into the vessel wall and exposed struts are easily snagged. Snagging on stent struts during crossing could dislodge the stent or release embolic material. An inability to cross the stent makes the removal of the filter device very hazardous. Complete visualisation of a retrieval catheter tip during stent crossing allows the user identify and evaluate potential stent snagging scenarios and modify their technique to aid advancement through the stent. Furthermore the radiopacifying technology of this invention allows the retrieval catheter tip to have a bull-nosed profile and this further reduces the risk of snagging.
Complete visualisation of the retrieval catheter tip when advancing it near a deployed filter helps prevent inadvertent filter engagement and dislodgement, with associated risk of embolic release.
When retrieving filters with very large embolic loads (>20 mg) the retrieval forces can increase considerably. If the tip is very rigid the force to retrieve could rupture the filter and release a large bolus of embolic material. Retrieval tips that are soft and conformable reduce the force of retrieval and reduce this risk. If the soft retrieval tip is highly radiopaque as per this invention then the probability of complications during this step is further reduced. The user can visualise the expansion of the tip on the fluoroscope and appreciate that the filter has captured a large bolus of embolic debris (embolic material is radiolucent). The user can thus identify problems and modify their actions accordingly, reducing risk of embolic loss and filter or retrieval catheter damage.
Another area of retrieval complications occurs when the distal parking space for the filter is small. In this scenario the filter is positioned just distal of the stent. A lot of skill is required to retrieve current filters in such situations as poor radiopacity of the devices makes it difficult to judge the exact position of the device. Retrieving a filter inside a stent is very hazardous as a stent strut can become entangled during the retrieval step. The radiopacity technology of this invention allows the distal margin of the retrieval catheter to be clearly identified thus minimising the risk of in stent retrieval. In-stent retrieval is a serious clinical risk as the filter may snag on the stent during retrieval and the relative force of retrieval may then cause device or vessel damage with release of captured emboli.

Typical values of x-ray energy used in interventional fluoroscopy ranges from 50 keV to 100 keV, it is this range that is of interest when evaluating interventional device radiopacity. In clinical use the x-ray photon energy may vary on a patient-by-patient basis, depending on: weight, application etc., In addition, the related image detection & enhancement technologies may also vary considerably by location. Therefore, the design of radiopaque elements in interventional medical devices must ensure sufficient x-ray attenuation across the 50 keV-100 keV range. A value of 70 keV has been found to be a reasonable comparison base to use.

Typically, the radiopaque materials used in medical devices are metal based—either in elemental, alloy or compound form. Metals typically exhibit x-ray attenuation “spikes”, within the normal interventional x-ray energy range. This “spike” is related to the atomic structure of the metal and corresponds to the binding energy of its innermost shell of electrons (K shell). The benefit in radiopacity terms is that these attenuation peaks can be used to improve imaging of these materials in interventional fluoroscopy. In one embodiment of this invention a radiopacifing filler material with an attenuation spike in the range 50 keV to 100 keV is employed. In another embodiment a radiopacifier with an attenuation spike at 70 keV is used.

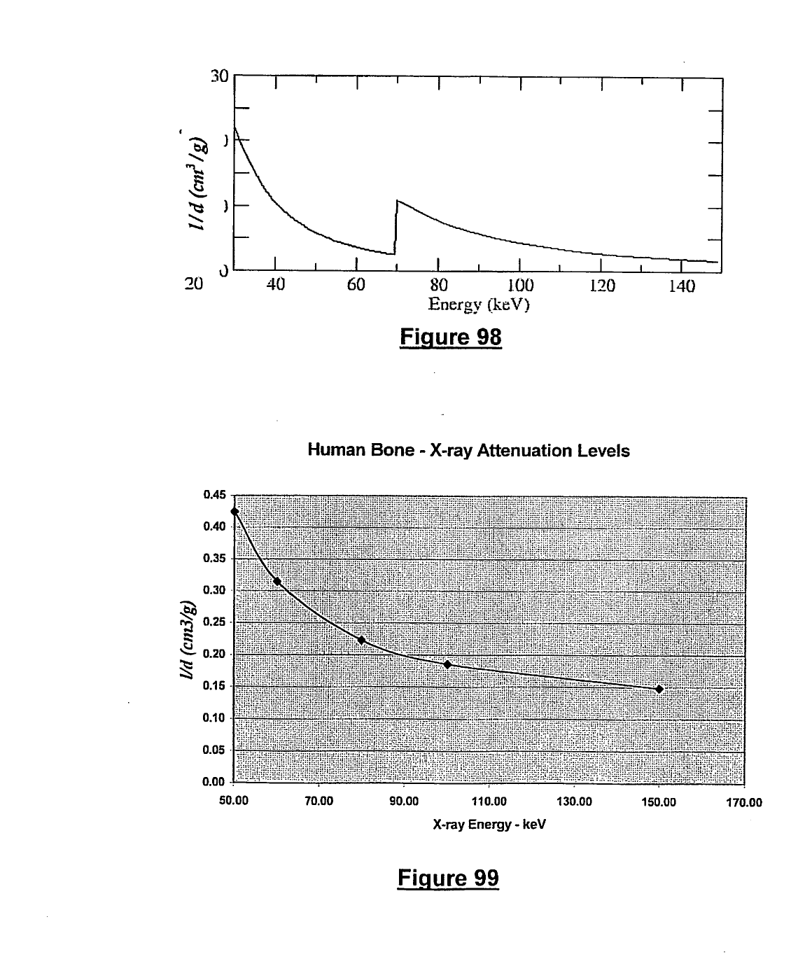

An example of the attenuation profile for elemental Tungsten is shown in FIG. 98. In this graph the attenuation spike at 70 keV can be seen. These spikes, also known as “absorption edges”, are characteristic to the metal type.

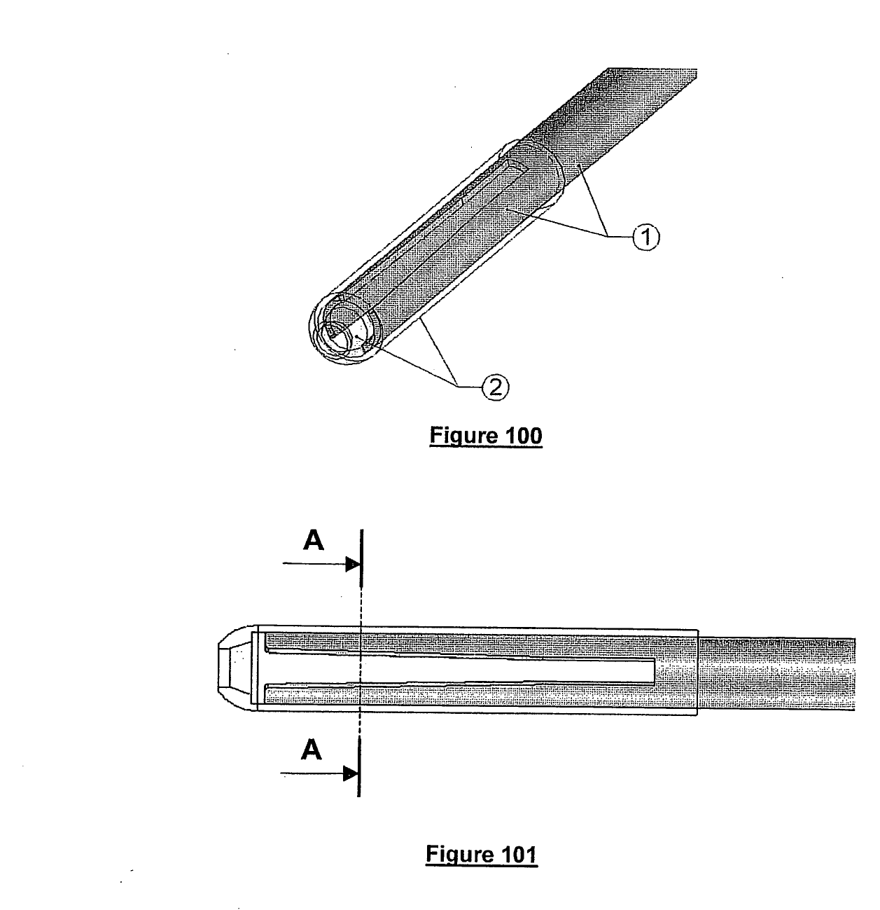

In contrast, polymeric materials have low attenuation levels similar to tissue, and water. Bone due to its higher density has moderate attenuation under x-ray. Bone, polymers and tissues do not typically exhibit absorption edges in the 50-100 keV x-ray energy range, having with attenuation profiles similar to the shape shown for bone in FIG. 99.

Polymer materials used in the tip construction do not significantly affect the tip radiopacity. However, the ability of these polymers to accommodate significant loading of radiopaque fillers without significant changes to their base polymer's mechanical characteristics is critical. Even at moderate filler loadings the stress-strain properties and UTS of the material can be dramatically changed. It is a goal of this invention to select polymers and particle sizes that can lead to minimal impact to the properties of the polymer. Ideally the polymer can accommodate filler loading of greater than 40%. More preferably the polymer can accommodate a loading of greater than 55%. More preferably the polymer can accommodate a loading of greater than 70%. Even more preferably the polymer can accommodate a loading of 80% or more.

In order to accommodate these high loadings selection of particulate size is very important. Large particles are difficult to process and can be points of failure initiation. Very small particles can interact more directly with the polymer chains and cause an increase in the modulus of the material. It will also be appreciated that a distribution of particle sizes exists and the tail ends of the distribution may be wide. Hence for the purposes of this invention particle size will be defined as it relates to 90% of the population of particles. Ideally 90% of the particles of the radiopacifier have a major axis of less than 20 microns. More ideally 90% of the particles of the radiopacifier have a major axis between 0.01 microns and 15 microns. More ideally 90% of the particles of the radiopacifier have a major axis between 1 microns and 10 microns. Most preferably 90% of the particles of the radiopacifier have a major axis between 1 microns and 4 microns.

### EXAMPLE 1

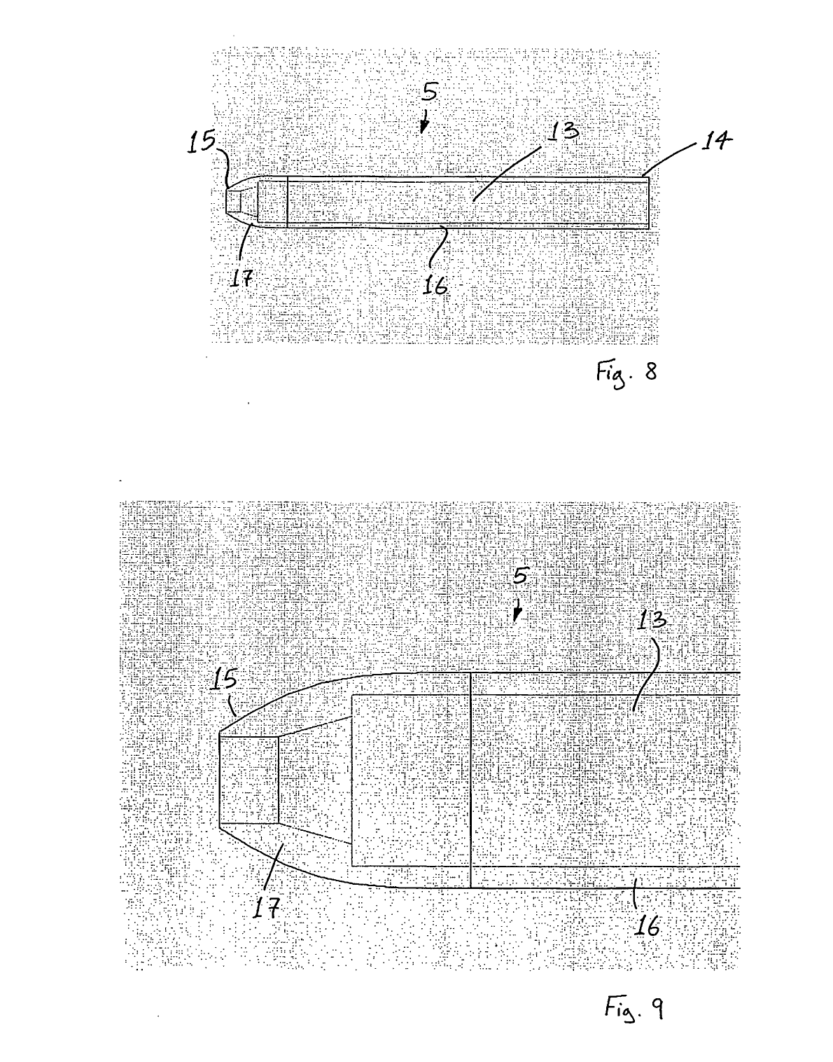

FIGS. 100-102 show a retrieval catheter tip of this invention. The manner in which the radiopacifying technology of this invention is applied to this tip will be described below. It will be appreciated that the radiopacifying technology of this invention could be applied to many other geometric constructions. This embodiment combines relatively high modulus polymer shaft and splines 1 and a low modulus shaped polymer cuff 2. The shaped polymer cuff 2 is a soft expansile material and it is joined to the splines 1. The soft cuff is radiopacified per this invention. The cuff 2 and splines 1 may be joined by a number of bonding, joining or fusing processes. In this example the cuff 2 and splines 1 are fused. The cuff 2 and splines 1 can expand in the radial direction. The soft material is loaded with a radiopacifier, in this instance tungsten. The tip is loaded with tungsten to 70% by weight. The polymer of the cuff 2 is a low modulus 45A S-EB-S (styrene-ethylene/butylene-styrene) with an ultimate elongation of >600%. The particle size of the filler material is in the range Ø0.001-Ø0.004 mm. This small particulate size will not create significant stress concentrators within the S-EB-S/tungsten matrix.
The tensile stress strain curve of the cuff 2 is shown in FIG. 8. It will be appreciated from FIG. 8 that the inclusion of 70% by weight of tungsten has not altered the properties of the soft material considerably. The fact that this loading does not alter the cuff 2 properties significantly allows the tip to be highly expansile. Also the distal end of the cuff 2 is profiled to provide a smooth transition to a filter delivery wire reducing the risk of tip snagging during advancement to the filter element site. The distal ID of the tip is much smaller than the wrapping profile of typical filter devices but can expand to capture the filters. This distal profile is very effective at tracking and crossing stents.

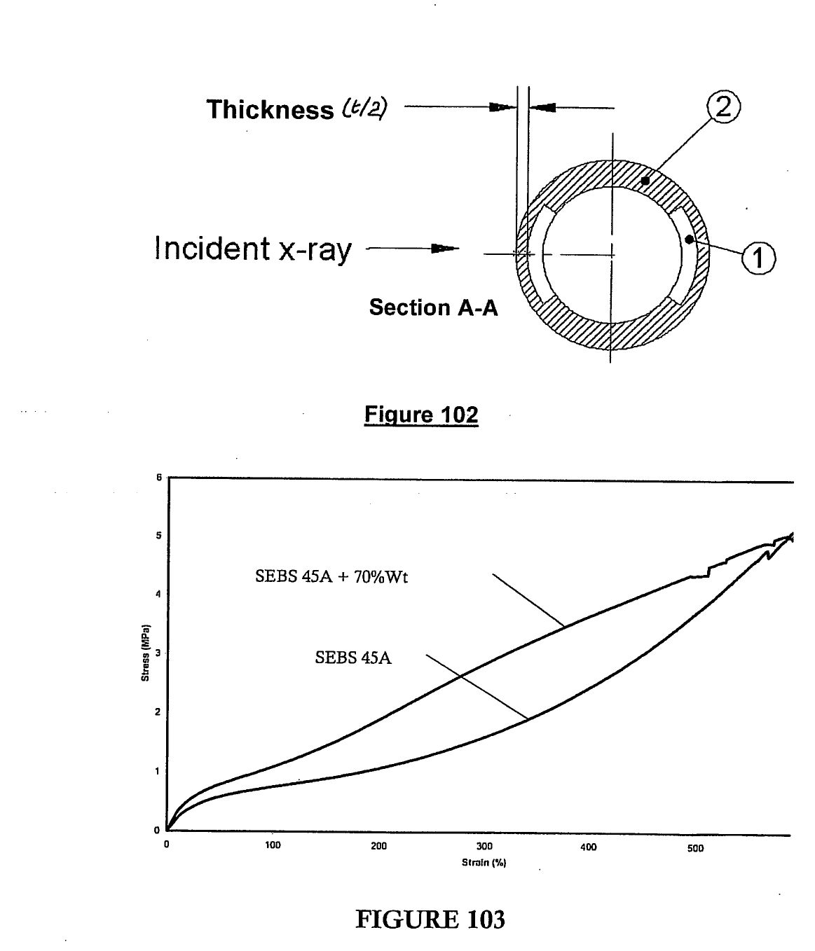

FIG. 101 shows a side elevation of the same embodiment denoting a section view A-A which is shown in schematic in FIG. 102.

While the invention is described in terms of this embodiment geometry it must be appreciated that the same inventive principles can also be applied to numerous other geometric embodiments.

In one embodiment the matrix polymer of the tip is a soft elastomeric material. Preferably this material has an ultimate elongation of greater than 200%. More preferably this material has an ultimate elongation of greater than 400%. Even preferably this material has an ultimate elongation of greater than 400%. Ideally the tip material has an ultimate elongation of greater than 200% with the radiopacifier loaded. More preferably the tip material has an ultimate elongation of greater than 400% with the radiopacifier loaded. Even more preferably the tip material has an ultimate elongation of greater than 600% with the radiopacifier loaded.

Ideally the radiopacified tip material is a soft material of low modulus compound. Preferably the secant modulus of the radiopacified tip material at 100% elongation is less than 2.0 MPa. More preferably the secant modulus of the radiopacified tip material at 100% elongation is less than 1.5 MPa. Even more preferably the secant modulus of the radiopacified tip material at 100% elongation is less than 1.0 MPa.

We have developed a metric that allows a numerical correlation between the formulation of the catheter compound and the radiopacity achieved on a fluoroscope of a particular energy. For the purpose of this invention and illustration all calculations will assume a fluoroscope energy level of 70 keV, a common energy level of catheterisation lab fluoroscopes. The metric is the mass attenuation thickness coefficient (MATC) and combines the linear attenuation properties of the compound components, the density of the compound components and the thickness of the tip. The thickness of the tip is defined as the sum of the wall thicknesses normal to the central axis of the tip. The MATC for a tip made from a homogeneous material is defined by:

$$( l / d ) \times t$$

Where l is the linear attenuation coefficient, d is the material density and t is the material thickness.

Per this invention the tip is a compound. Hence the formula above needs to be modified to add the components of the compound. The MATC for the compounded tip of this invention is:

$${( l / d )}_{top} \times t = \{ {{w}_{pol} ( l / d )}_{pol} + {{w}_{rad} ( l / d )}_{rad} \} \times t$$

Where Wₚₒₗ₌weight percentage of compound due to polymer
Where W_{Rad=}weight percentage of compound due to radiopacifier

The MATC parameter is applied especially to catheter tips and has the advantage that direct comparisons can be made between soft polymer tips and marker bands. This allows great prediction as to the performance of a tip in vivo.

The MATC for the tips of this invention at 70 keV are preferably greater than >0.0 cm³/g. More preferably the MATC for the tip is greater than >0.2 cm³/g. More preferably the MATC is greater than 0.25 cm³/g. More preferably the MATC is greater than 0.30 cm³/g.

### EXAMPLE 2

In this example the MATC for the tip of example 1 will be calculated. The value of l (linear attenuation coefficient) varies with x-ray photon energy, hence the MATC value also varies with photon energy. As described, the photon energy range for interventional x-rays is 50 keV to 100 keV. For the purposes of comparitive disclosure a x-ray photon energy value of 70 keV will be used.
From published information on l and d, the MATC value, for the preferred tip embodiment at 70 keV, can be calculated as:

$${MATC}_{tip} = \{ 0.3 {( l / d )}_{S - EB - S} + 0.70 {( l / d )}_{Tungsten} \} \times 0.04 cm$$

$${MATC}_{tip} = \{ 0.3 {( 0.2 )}_{S - EB - S} + 0.70 {( 11 )}_{Tungsten} \} \times 0.04 cm$$

$${MATC}_{tip} = 0.311 {cm}^{3} \text{/} g$$

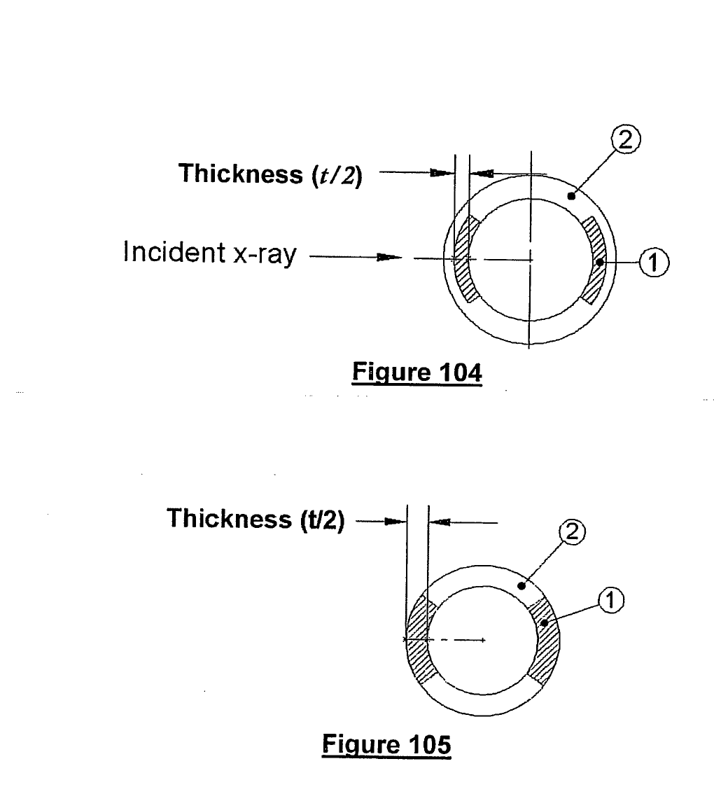

In an further embodiment of the design, the radiopaque filler is added to the stiffer polymer component (1). In this design the radiopaque filler is compounded with the polymer used to produce the shaft and splines. A schematic of a section A-A through this type of construction is shown in FIG. 104

In a further embodiment of the design the radiopaque filler section and the unfilled section are arranged as shown in FIG. 104. In this arrangement the materials have minimum overlap with each other. In this embodiment the radiopaque filler is compounded with stiffer polymer material.

In a further embodiment of the design the radiopaque filler section and the unfilled section are arranged as shown in FIG. 105. This arrangement is similar to FIG. 104, but in this embodiment the radiopaque filler is compounded with the expansile polymer material (2).

In alternate embodiments of the design the type of radiopaque filler compounded with a tip polymer element may vary from Tungsten formed instead from Gold (Au), Tantelum (Ta), Platinum (Pt), Iridium (Ir) and Niobium (Ni) or mixtures of these components.

In another embodiment the radiopaque filler is an element with an atomic number greater than 56. More preferably the atomic number is greater than 70. More preferably the atomic number of the radiopacifier is between 73 and 83. It will be appreciated that metals with excellent biocompatibility are preferred. Non-biocompatible radiopacifiers need to be appropriately shielded from blood contact. Some radiopacifiers i.e. Bismuth (Bi) atomic number 83 and Barium (Ba) atomic number 56 are compounded with elements before use.

In another embodiment the radiopaque filler is composed of elements belonging to Periodic Table Groups 4-11 and Periodic Table Periods 4-6. More preferably they belong to Groups 4-6, and 9-11 and Period 6.

In a further embodiment a mixture of two or more particulate types may be used, this would allow more than one attenuation peak to be incorporated into the attenuation profile of the tip material over the 50 keV-100 keV range.

In a further embodiment particulate created from an alloy of two or more metals may by used as the radiopaque filler. For example, CoCr, Pt Ir etc.

It will be appreciated that more than two reinforcement columns may be provided. The number of columns may be chosen to suit the requirements of the intended use of the retrieval catheter.

Furthermore it will be appreciated that the retrieval catheter is suitable for retrieving objects other than embolic protection filters. For example, the retrieval catheter of the invention is suitable for retrieving calculi, such as a gallstone.

The invention is not limited to the embodiments hereinbefore described, with reference to the accompanying drawings, which may be varied in construction and detail.

1. A retrieval catheter tip comprising:—
an expandable tip body into which an object may be retrieved;
the tip body comprising a relatively stiff segment and a relatively soft segment; and
at least one reinforcement element for reinforcing the tip body.

2. A tip as claimed in claim 1 wherein the tip body is at least partially of a radiopaque material.

3. A tip as claimed in claim 2 wherein the stiff segment is at least partially of a radiopaque material.

4. A tip as claimed in claim 1 wherein the stiff segment and the soft segment are formed separately.

5. A tip as claimed in claim 1 wherein the stiff segment and the soft segment are bonded together.

6. A tip as claimed in claim 1 wherein the stiff segment is located distally of the soft segment.

7. A tip as claimed in claim 1 wherein the segment extends around the full circumference of the tip.

8. A tip as claimed in claim 1 wherein the segment is substantially tubular.

9. A retrieval catheter tip comprising:—
an expandable tip body into which an object may be retrieved; and
at least one reinforcement element for reinforcing the tip body;
the tip body comprising a first segment along the at least one reinforcement element and a second segment distally of the at least one reinforcement element;
Area 1 being less than Area 2;
where: Area 1 is the area of tip body material at a cross-section in the first segment; and
Area 2 is the area of tip body material at a cross-section in the second segment.

10. A tip as claimed in claim 9 wherein the tip body has a reception space into which an object may be retrieved.

11. A tip as claimed in claim 10 wherein the radial dimension of the reception space varies along at least part of the length of the reception space.

12. A tip as claimed in claim 11 wherein at least part of the reception space tapers distally from a larger radial dimension to a smaller radial dimension.

13. A tip as claimed in claim 9 wherein the wall thickness of the tip body varies along at least part of the length of the tip body.

14. A tip as claimed in claim 13 wherein at least part of the tip body tapers distally from a smaller wall thickness to a larger wall thickness.

15. A tip as claimed in claim 9 wherein the wall thickness of the tip body is constant along at least part of the length of the tip body.

16. A tip as claimed in claim 13 wherein the wall thickness of the tip body is constant along the first segment and varies along the second segment.

17. A tip as claimed in claim 9 wherein the tip is shaped for a smooth crossing profile.

18. A tip as claimed in claim 17 wherein a distal end of the tip is rounded.

19. A tip as claimed in claim 18 wherein a distal end of the tip body is rounded.

20. A retrieval catheter tip suitable for exchange over a guidewire, the tip comprising:—
an expandable tip body;
the tip body having a reception space into which an object may be retrieved;
the tip body comprising a distal opening through which the object may enter the reception space;
the radial dimension of the distal opening being less than substantially three times the radial dimension of a guidewire over which the tip is exchangeable.

21. A tip as claimed in claim 20 wherein the radial dimension of the distal opening is approximately equal to two times the radial dimension of a guidewire over which the tip is exchangeable.

22. A tip as claimed in claim 20 wherein the radial dimension of the distal opening is approximately 0.030″ (0.762 mm).

23. A tip as claimed in claim 20 wherein the radial dimension of the reception space varies along at least part of the length of the reception space.

24. A tip as claimed in claim 23 wherein at least part of the reception space tapers distally from a larger radial dimension to a smaller radial dimension.

25. A tip as claimed in claim 20 wherein the tip is shaped for a smooth crossing profile.

26. A tip as claimed in claim 25 wherein a distal end of the tip is rounded.

27. A tip as claimed in claim 26 wherein a distal end of the tip body is rounded.

28. A retrieval catheter tip comprising:—
an expandable tip body into which an object may be retrieved; and
at least one reinforcement element for reinforcing the tip body.

29. A tip as claimed in claim 28 wherein the reinforcement element comprises a reinforcement column extending along at least part of the length of the tip body.

30. A tip as claimed in claim 29 wherein the reinforcement column extends along only part of the length of the tip body.

31. A tip as claimed in claim 29 wherein the reinforcement column comprises a spline.

32. A tip as claimed in claim 28 wherein the circumferential dimension of the reinforcement element varies along at least part of the length of the reinforcement element.

33. A tip as claimed in claim 32 wherein at least part of the reinforcement element tapers distally from a larger circumferential dimension to a smaller circumferential dimension.

34. A tip as claimed in claim 33 wherein the larger circumferential dimension is in the range of from 120° to 170°.

35. A tip as claimed claim 33 wherein the smaller circumferential dimension is in the range of from 60° to 120°.

36. A tip as claimed in claim 32 wherein the circumferential dimension of the reinforcement element varies along the entire length of the reinforcement element.

37. A tip as claimed in claim 28 wherein the circumferential dimension of the reinforcement element is constant along at least part of the length of the reinforcement element.

38. A tip as claimed in claim 37 wherein the circumferential dimension of the reinforcement element is constant along the entire length of the reinforcement element.

39. A tip as claimed in claim 32 wherein the circumferential dimension of the reinforcement element is constant along a first part of the length of the reinforcement element and varies along a second part of the length of the reinforcement element.

40. A tip as claimed in claim 39 wherein the first part is located proximally of the second part.

41. A tip as claimed in claim 28 wherein a distal end of the reinforcement element is rounded.

42. A tip as claimed in claim 28 wherein the tip comprises two reinforcement elements spaced-apart circumferentially around the tip.

43. A tip as claimed in claim 42 wherein the reinforcement elements diametrically oppose one another.

44. A tip as claimed in claim 28 wherein the tip body extends over an outer surface of the reinforcement element.

45. A tip as claimed in claim 28 wherein the tip body extends over an inner surface of the reinforcement element.

46. A tip as claimed in claim 28 wherein the tip body extends between laterally adjacent reinforcement elements.

47. A tip as claimed in claim 28 wherein at least part of the reinforcement element is embedded within at least part of the tip body.

48. A tip as claimed in claim 47 wherein the reinforcement element is embedded within the tip body along only part of the length of the reinforcement element.

49. A tip as claimed in claim 47 wherein the reinforcement element is embedded within the tip body along substantially the full length of the reinforcement element.

50. A tip as claimed in claim 28 wherein the tip body extends distally of a distal end of the reinforcement element.

51. A tip as claimed in claim 28 wherein the tip body has a reception space into which an object may be retrieved.

52. A tip as claimed in claim 51 wherein the radial dimension of the reception space varies along at least part of the length of the reception space.

53. A tip as claimed in claim 52 wherein at least part of the reception space tapers distally from a larger radial dimension to a smaller radial dimension.

54. A tip as claimed in claim 52 wherein at least part of the reception space tapers distally from a smaller radial dimension to a larger radial dimension.

55. A tip as claimed in claim 51 wherein the radial dimension of the reception space is constant along at least part of the length of the reception space.

56. A tip as claimed in claim 52 wherein the radial dimension of the reception space is constant along a first part of the length of the reception space and varies along a second part of the length of the reception space.

57. A tip as claimed in claim 56 wherein the first part is located proximally of the second part.

58. A tip as claimed in claim 28 wherein the wall thickness of the tip body varies along at least part of the length of the tip body.

59. A tip as claimed in claim 58 wherein at least part of the tip body tapers distally from a smaller wall thickness to a larger wall thickness.

60. A tip as claimed in claim 58 wherein at least part of the tip body tapers distally from a larger wall thickness to a smaller wall thickness.

61. A tip as claimed in claim 28 wherein the wall thickness of the tip body is constant along at least part of the length of the tip body.

62. A tip as claimed in claim 58 wherein the wall thickness of the tip body is constant along a first part of the length of the tip body and varies along a second part of the length of the tip body.

63. A tip as claimed in claim 62 wherein the first part is located proximally of the second part.

64. A tip as claimed in claim 28 wherein the tip is shaped for a smooth crossing profile.

65. A tip as claimed in claim 64 wherein a distal end of the tip is rounded.

66. A tip as claimed in claim 65 wherein a distal end of the tip body is rounded.

67. A tip as claimed in claim 28 wherein at least part of the tip body is coupled to at least part of the reinforcement element.

68. A tip as claimed in claim 67 wherein at least part of the tip body is attached to at least part of the reinforcement element.

69. A tip as claimed in claim 68 wherein the tip body is attached to the reinforcement element along only part of the length of the reinforcement element.

70. A tip as claimed in claim 68 wherein the tip body is attached to the reinforcement element along substantially the full length of the reinforcement element.

71. A tip as claimed in claim 68 wherein the tip body is attached to the reinforcement element around only part of the circumferential dimension of the reinforcement element.

72. A tip as claimed in claim 68 wherein the tip body is attached to the reinforcement element around substantially the full circumferential dimension of the reinforcement element.

73. A tip as claimed in claim 28 wherein the tip body is detached from the reinforcement element.

74. A tip as claimed in claim 68 wherein the tip body is heat-sealed to the reinforcement element.

75. A tip as claimed in claim 28 wherein the tip comprises a separator to separate laterally adjacent reinforcement elements.

76. A tip as claimed in claim 75 wherein the separator is provided on the tip body.

77. A tip as claimed in claim 76 wherein the separator comprises a projection projecting from a surface of the tip body.

78. A tip as claimed in claim 77 wherein the projection projects radially inwardly from an inner surface of the tip body.

79. A tip as claimed in claim 77 wherein a circumferential dimension of the projection varies along at least part of the length of the projection.

80. A tip as claimed in claim 79 wherein the projection tapers distally from a smaller circumferential dimension to a larger circumferential dimension.

81. A tip as claimed in claim 28 wherein the reinforcement element extends proximally of a proximal end of the tip body.

82. A tip as claimed in claim 28 wherein the reinforcement element terminates at a proximal end of the tip body.

83. A tip as claimed in claim 28 wherein the tip comprises an expandable member.

84. A tip as claimed in claim 83 wherein the expandable member extends around the reinforcement element.

85. A tip as claimed in claim 83 wherein the expandable member comprises a band.

86. A tip as claimed in claim 83 wherein the expandable member is radiopaque.

87. A tip as claimed in claim 83 wherein the longitudinal dimension of the expandable member is small relative to the longitudinal dimension of the tip body and/or of the reinforcement element.

88. A tip as claimed in claim 28 wherein the tip comprises a limiter to limit the extent of expansion of the tip body.

89. A tip as claimed in claim 88 wherein the limiter is movable from a radially collapsed configuration to a radially expanded configuration.

90. A tip as claimed in claim 89 wherein in the radially expanded configuration the limiter is configured to resist any further expansion.

91. A tip as claimed in claim 90 wherein the limiter comprises a mechanical lock to resist any further expansion.

92. A tip as claimed in claim 88 wherein the limiter extends around the tip body.

93. A tip as claimed in claim 88 wherein the limiter comprises a band.

94. A tip as claimed in claim 88 wherein the longitudinal dimension of the limiter is small relative to the longitudinal dimension of the tip body and/or of the reinforcement element.

95. A retrieval catheter comprising a catheter shaft and a retrieval catheter tip as claimed in claim 28 at a distal end of the shaft.

96. A catheter as claimed in claim 95 wherein the tip is fixed to the distal end of the shaft.

97. A catheter as claimed in claim 96 wherein the reinforcement element is fixed to the distal end of the shaft.

98. A catheter as claimed in claim 97 wherein the reinforcement element is formed integrally with the shaft.

99. A catheter as claimed in claim 95 wherein the proximal end of the tip body is spaced distally of the distal end of the catheter shaft.

100. A catheter as claimed in claim 95 wherein the outer diameter of the tip body is substantially equal to the outer diameter of the catheter shaft.

101. A retrieval catheter tip comprising:
an expandable tip body into which an object can be retrieved; and
at least one reinforcement element for reinforcing the tip body;
the tip comprising a soft polymer filled with radiopacifier,
the tip having a material attenuation thickness coefficient (MATC) as hereinbefore defined of greater than or equal to 0.10 cm³/g.

102. A retrieval catheter tip as claimed in claim 101 wherein the MACT is greater than 0.2 cm³/g.

103. A retrieval catheter tip as claimed in claim 101 wherein the MACT is greater than 0.25 cm³/g.

104. A retrieval catheter tip as claimed in claim 101 wherein the MACT is greater than 0.3 cm³/g.

105. A retrieval catheter tip as claimed in claim 101 wherein the rapiopacifier is at least partially in particulate form and at least 90% of the particles have a major dimension which is less than 20 microns in length.

106. A retrieval catheter tip as claimed in claim 105 wherein the major dimension is from 0.01 to 15 microns in length.

107. A retrieval catheter tip as claimed in claim 105 wherein the major dimension is from 0.01 to 15 microns in length.

108. A retrieval catheter tip as claimed in claim 105 wherein the major dimension is from 1 to 10 microns in length.

109. A retrieval catheter tip as claimed in claim 105 wherein the major dimension is from 1 to 4 microns in length.

110. A retrieval catheter tip as claimed in claim 101 wherein the radiopaque filler is an element with an atomic number greater that 56.

111. A retrieval catheter tip as claimed in claim 101 wherein the radiopaque filler is an element with an atomic number greater than 70.

112. A retrieval catheter tip as claimed in claims 101 wherein the radiopaque filler is an element with an atomic number of from 73 to 83.

113. A retrieval catheter tip as claimed in claim 101 wherein the radiopaque filler comprises an element belonging to periodic table group 4 to 11.

114. A retrieval catheter tip as claimed in claim 101 wherein the radiopaque filler comprises an element belonging to periodic table period 4 to 6.

115. A retrieval catheter tip as claimed in claim 101 wherein the radiopaque filler comprises an element belonging to periodic table group 4 to 6 and period 6.

116. A retrieval catheter tip as claimed in claim 101 wherein the radiopaque filler comprises Tungsten.

117. A retrieval catheter tip as claimed in claim 101 wherein the secant modulus of the radiopacified tip material at 100% elongation is less than 2.0 MPa.

118. A retrieval catheter tip as claimed in claim 101 wherein the secant modulus of the radiopacified tip material at 100% elongation is less than 1.5 MPa.

119. A retrieval catheter tip as claimed in claim 101 wherein the secant modulus of the radiopacified tip material at 100% elongation is less than 1.1 MPa.

120. A retrieval catheter tip as claimed in claim 101 wherein the tip material loaded with radiopacifier has an ultimate elongation of greater than 200%.

121. A retrieval catheter tip as claimed in claim 101 wherein the tip material loaded with radiopacifier has an ultimate elongation of greater than 400%.

122. A retrieval catheter tip as claimed in claim 101 wherein the tip material loaded with radiopacifier has an ultimate elongation of greater than 600%.

123. A retrieval catheter tip as claimed in claim 101 wherein the radiopacifier is present in an amount of greater than 40% of the polymer material.

124. A retrieval catheter tip as claimed in claim 101 wherein the radiopacifier is present in an amount of greater than 55% of the polymer material.

125. A retrieval catheter tip as claimed in claim 101 wherein the radiopacifier is present in an amount of greater than 70% of the polymer material.

126. A retrieval catheter tip as claimed in claim 101 wherein the radiopacifier is present in an amount of greater than 80% of the polymer material.
